(19) United States
(12) Patent Application Publication
(10) Pub. No.: US 2025/0378939 A1
(43) Pub. Date: Dec. 11, 2025

(54) AN INTRAORAL SCANNING DEVICE CONFIGURED TO AUTHENTICATE MODE REQUEST

(71) Applicant: 3SHAPE A/S, Copenhagen K (DK)

(72) Inventor: Anders Robert JELLINGGAARD, Copenhagen K (DK)

(73) Assignee: 3SHAPE A/S, Copenhagen K (DK)

(21) Appl. No.: 18/875,766

(22) PCT Filed: Jun. 16, 2023
(86) PCT No.: PCT/EP2023/066209

§ 371 Date: Dec. 17, 2024

(30) Foreign Application Priority Data
Jun. 17, 2022 (EP) 22179522.2

(51) Int. Cl. G16H 30/20; G16H 10/65; H04L 9/32; H04N 23/50; H04N 23/66; H04N 23/667

(52) U.S. Cl. CPC G16H 30/20; G16H 10/65; H04L 9/3247; H04N 23/555; H04N 23/66; H04N 23/667

(57) ABSTRACT

An intraoral scanning device is configured to acquire intraoral scan data from a three-dimensional dental object. The intraoral scanning device includes a processing unit configured to process intraoral scan data and provide 2D image data and/or 3D image data, a memory, and a wireless interface configured to transmit the 2D image data and/or the 3D image data, wherein the processing unit is configured to receive a mode request via the wireless interface, the mode request is one or more of a service mode request for a service mode, a customization mode request, an upgrade mode request and a debug mode request, wherein the service mode is characterized in that a firmware part of the memory is writable, authenticate the mode request; and place the intraoral scanning device into the requested mode if authentication of the mode request succeeds.

### FIELD

The present disclosure relates to an intraoral scanning device and in particular to intraoral scanning device and related method for configuration or operation of an intraoral scanning device.

### BACKGROUND

The functionality of an intraoral scanning device becomes increasingly advanced. Wireless communication between an intraoral scanning device and external devices, such as a clinic computer, a scan computer, a dental software on a computer, and a customization computer, has evolved. Typically, a wireless communication interface of an intraoral scanning device uses open standard-based interface. However, this poses many challenges in terms of security. An intraoral scanning device may assume any incoming data as legitimate, and may allow memory to be written or changed by an unauthorized party. Any such attacks may result in a malfunction of the intraoral scanning device, or a battery exhaustion attack.

However, an intraoral scanning device is a small device with strict constraints in terms of computational power, memory space, etc. Therefore, a device communicating with an intraoral scanning device cannot use an off-the-shelf security algorithm and protocol, at the risk of e.g. depleting the intraoral scanning device battery or degrading functions of the intraoral scanning device rendering the intraoral scanning quasi-useless.

Present intraoral scanning devices are part of a service infrastructure which includes communication between intraoral scanning devices, scan software for a specific service, and the provider of the service. The service could for example include manufacture of an aligner, a retainer, a crown, an implant, a bracer, a nightguard etc. For improving the usability of such an infrastructure for the dentist, minimal interaction between the infrastructure and the dentist is needed. One way of achieving this is by applying wireless communication between the intraoral scanning device and an external computer that is connected to a server that can forward the intraoral scan data to a service provider. Scan data of a patient can be characterized as being personal information, and therefore, there is a need for minimizing any risk of a third party stealing or corrupting the at least scan data. The scan data is characterized as personal information, and in some situations, other type of personal information is associated with the scan data, such as age, gender, location address, personal security number etc. In this example, a demand for improving the security of the wireless communication in the service infrastructure is needed.

### SUMMARY

An aspect of the present disclosure is to reduce risk of a third party accessing any part of the intraoral scanning device. There is a need for an intraoral scanning device that is protected against unauthorized modification of the intraoral scanning device and operation thereof.

A further aspect of the present disclosure is to provide an intraoral scanning device, and a method which seeks to mitigate, alleviate, or eliminate a third party's possibility to steal and/or corrupt personal information of the patient.

Yet another aspect of the present disclosure is to improve security of an intraoral scanning device. Security comprises in assessing threats, vulnerabilities and attacks and developing appropriate safeguards and countermeasures to protect against threats and attacks. The present disclosure relates to an intraoral scanning device comprising a processing unit configured to process intraoral scan data of a patient and provide 2D image data and/or 3D image data.

According to the aspect, a handheld intraoral scanning device for acquiring intraoral scan data from a three-dimensional dental object during a scanning session is disclosed. The handheld intraoral scanning device may comprise a processing unit configured to process intraoral scan data of a patient and provide 3D image data: a memory; and a wireless interface configured to transmit the 3D image data, wherein the processing unit is configured to receive a mode request via the wireless interface when no 3D image data is being transmitted, wherein the mode request is one or more of a service mode request for a service mode, a customization mode request for customizing a user interface of the handheld intraoral scanning device, an upgrade mode for upgrading the handheld intraoral scanning device and a debug mode request, wherein the service mode is characterized in that a firmware part of the memory is writable: authenticate the mode request to confirm that the mode request is valid for the handheld intraoral scanning device; and place the handheld intraoral scanning device into the requested mode if authentication of the mode request succeeds.

According to the aspect, a method for configuration of a haneheld intraoral scanning device that may comprise a processing unit configured to process intraoral scan data of a patient and provide 3D image data is discloses. The handheld intraoral scanning device may further include a memory unit and a wireless interface configured to transmit the 3D image. The method may comprise receiving a mode request via the wireless interface when no 3D image data is transmitted, wherein the mode request may be one or more of a service mode request for updating firmware data, a customization mode request, an upgrade mode request and a debug mode request, and wherein the service mode implies that a firmware part of the memory is writable. Furthermore, the method may comprise authenticating the mode request to confirm that the mode request is valid for the handheld intraoral scanning device, and placing the intraoral scanning device into the requested mode if authentication of the mode request succeeds.

According to the aspect, an intraoral scanning device for acquiring intraoral scan data from a three-dimensional dental object during a scanning session is disclosed. The intraoral scanning device may comprise a processing unit configured to process intraoral scan data of a patient: a memory; and a wireless interface configured to transmit the intraoral scan data of the patient, wherein the processing unit is configured to receive a mode request via the wireless interface, wherein the mode request is one or more of a service mode request for a service mode, a customization mode request, an upgrade mode and a debug mode request, wherein the service mode is characterized in that a firmware part of the memory is writable: authenticate the mode request; and place the intraoral scanning device into the requested mode if authentication of the mode request succeeds.

According to the aspect, an intraoral scanning device for acquiring intraoral scan data from a three-dimensional dental object during a scanning session is disclosed. The intraoral scanning device may comprise a processing unit configured to process intraoral scan data of a patient: a memory; and a wireless interface configured to transmit the intraoral scan data of the patient, wherein the processing unit is configured to receive a mode request via the wireless interface, wherein the mode request is one or more of a service mode request for a service mode, a customization mode request, an upgrade mode and a debug mode request, wherein the service mode is characterized in that a part of the memory is writable: authenticate the mode request; and place the intraoral scanning device into the requested mode if authentication of the mode request succeeds.

According to the aspect, an intraoral scanning device for acquiring intraoral scan data from a three-dimensional dental object during a scanning session is disclosed. The intraoral scanning device may comprise a processing unit configured to process intraoral scan data of a patient and provide 2D image data and/or 3D image data: a memory; and a wireless interface configured to transmit the 2D image data and/or the 3D image data, wherein the processing unit is configured to receive an instruction request via the wireless interface, wherein the instruction request is one or more of a service instruction request for a service instruction, a customization instruction request, an upgrade instruction request and a debug instruction request, wherein the service instruction is characterized in that a firmware part of the memory is writable: authenticate the instruction request; and place the intraoral scanning device into the requested instruction if authentication of the instruction request succeeds.

A mode request may be similar to an instruction request. For example, during transmission of data packages via the wireless communication link to the intraoral scanning device, each data package is being authenticated or verified based on a signature, and when all data packages are being successful authenticated or verified then the intraoral scanning mode is placed into a service instruction which results in installation of the data packages into the firmware part of the memory.

According to the aspect, an intraoral scanning device for acquiring intraoral scan data from a three-dimensional dental object during a scanning session is disclosed. The intraoral scanning device may comprise a processing unit configured to process intraoral scan data of a patient and provide 2D image data and/or 3D image data: a memory; and a wireless interface configured to transmit the 2D image data and/or the 3D image data, wherein the processing unit is configured to receive a connection request via the wireless interface, wherein the connection request is one or more of a service connection request for a service connection, a customization connection request, an upgrade connection request and a debug connection request, wherein the service connection is characterized in that a firmware part of the memory is writable; authenticate the connection request; and place the intraoral scanning device into the requested connection if authentication of the connection request succeeds.

A mode request, an instruction request, a connection request, a memory request, or a state request may be similar but with few distinguishing elements in relation to how the intraoral scanning device is being configured to receive data packages from an external device and install the data packages.

The handheld intraoral scanning device may receive the mode request when no 3D image data is being transmitted via the wireless interface. The

The intraoral scanning device may be placed into a requested mode which configures the intraoral scanning device to perform changes to how the images are being acquired by the optical unit, and how the processing unit is processing the images into image data, such as 2D image and/or 3D image.

An intraoral scanning device is in a scanning session when it is being used intentionally, such as for scanning an oral cavity of a patient.

The intraoral scanning device may be a handheld scanning device for scanning inside an oral cavity of a patient. The intraoral scanning device differs from other type of teeth scanning devices in that the intraoral scanning device is a handheld scanning device which can easily be handled by one hand by a user, and which has no wired connection to any external device during scanning of an inside of an oral cavity of a patient. Therefore, the only attack which an intraoral scanning device may experience is via the wireless interface.

The intraoral scanning device refers to a device configured to conduct a scan inside the oral cavity of a patient, or a part thereof, or parts thereof, such as a tooth, teeth, gingiva, etc., or to obtain a 2D image data and/or 3D image data of the oral cavity of a patient or parts thereof, such as a tooth, teeth and/or gingiva, etc. the intraoral scanning device may be an intraoral scanner that is fully or partly inserted in the oral cavity of a patient, such as a wireless intraoral scanning device.

The method and the intraoral scanning device as disclosed provide secure configuration of the intraoral scanning device, such as secure access to the memory of the intraoral scanning device. It is an advantage of the present disclosure that the intraoral scanning device can only be configured or updated by authorized parties. The disclosed intraoral thus has the advantage of detecting and preventing any modification by unauthorized parties. The intraoral scanning device disclosed herein is advantageously protected against attacks such as spoofing attacks, man-in-the-middle attacks, and/or replay-attacks.

The intraoral scanning device is the key element in providing the needed level of security in wireless communication in a service infrastructure which at least includes the intraoral scanning device and a scan computer or a dental software on a computer. It would not be possible for a third party to attack the wireless communication as this person needs to have the intraoral scanning device physically in its hand. It would not even be enough to have access to the scan computer or the dental software.

The method as disclosed herein provides a secure configuration and/or update of an intraoral scanning device.

The present disclosure provides improved security of an intraoral scanning device. Security comprises assessing threats, vulnerabilities and attacks and developing appropriate safeguards and countermeasures to protect against threats and attacks.

The intraoral scanning device comprises a processing unit. The processing unit may be configured to process intraoral scan data of a patient and provide 2D image data and/or 3D image data. The 2D image data and/or 3D image data may include information about the anatomy of the oral cavity of the patient, such as teeth, gingival, bone level, and/or information about diagnostic indicators such as caries, bone loss, gingivitis, gingiva recession, periodontitis, bone loss, cracks, and occlusion.

The 2D image data and/or the 3D image data may be image data configured to be visualizable on a display in a 2D or a 3D manner, respectively.

The intraoral scanning device may be operated in one or more modes. The one or more modes may include a first mode and/or a second mode. The one or more modes may include a third mode and/or a fourth mode. The one or more modes may include a default mode.

The first mode may be a service mode. A service mode may be characterized in that a firmware part of the memory can be written in the service mode. The firmware part of the memory may be write-protected in at least one other mode of the intraoral scanning device. Furthermore, the service mode may include setting the intraoral scanning device in a state where the optical unit of the intraoral scanning device is preparing to be used, for example, by heating up the light projector(s) and/or turning on the image sensor. Furthermore, the service mode may include setting the intraoral scanning device in a state where the intraoral scanning device is performing a self-check of moving parts, such as a moveable focus lens, an intensity of the light projector(s) and/or signal-to-noise of the image sensor. Other elements of the intraoral scanning device could be susceptible for a self-check but is not mentioned in this disclosure.

The second mode may be a customization mode. A customization mode may be characterized in that a customization part of the memory can be read and/or written in the customization mode. A customization mode may be characterized in that a firmware part of the memory is write-protected. The customization part of the memory may comprise setting data, such as power management settings, configuration of a user interface of the intraoral scanning device and/or settings of an optical unit of the intraoral scanning device. An intraoral scanning device may include a color image sensor, such as an RGB image sensor, and in the customization mode, different color areas may be configured to be deactivated and/or activated during at least a scanning session. Thus, the customization part of the memory may comprise data that relates to which color areas of the RBG image sensor should be activated or deactivated during a scanning session. An intraoral scanning device may include a monochromatic image sensor and colored light emitting diodes, and in the customization mode, the different colored light emitting diodes may be configured to be deactivated and/or activated during a scanning session. Thus, the customization part of the memory may comprise data that relates to which colored light emitting diodes should be activated or deactivated during a scanning session. A colored light emitting diode may be configured to emit light with a color, such as blue, red, green etc. In another example, the intraoral scanning device could include one or more near-infrared light emitting diodes which also can be set to be activated and/or deactivated during a scanning session in the customization mode.

The optical unit may include one or more light projectors, one or more optical components, and one or more image sensors.

The user interface of the intraoral scanning device may include at least a touch sensor, at least a touch button, at least a light emitting diode, a haptic sensor, and/or an accelerometer. The handheld intraoral scanning device may include a motion sensor which is configured to sense the motion of the handheld intraoral scanning device. The handheld intraoral scanning device is configured to communicate wirelessly with an external device that is connected to a display. A cursor on the display may be moved around based on motion signals provided by the motion sensor to the external device. The user is able to navigate the cursor on the display by moving the handheld intraoral scanning device. The service mode request may include settings update that relates to the motion sensor of the handheld intraoral scanning device, and the customization mode request may relate to a customization of a user interface of the handheld intraoral scanning device which may involve a graphical setup of a graphical user interface on the display. For example, when the handheld intraoral scanning device connects to the external device, the handheld intraoral scanning device forwards a customization package to the external device via the wireless interface, and the external device is then configured to change the graphical setup based on the customization package. The customization package may be updated by the customization mode request.

The third mode may be a debug mode. A debug mode may be characterized in that a debug part of the memory can be read and/or written in the customization mode. A debug mode may be characterized in that a customization part of the memory can be read and/or written in the debug mode. A debug mode may be characterized in that a firmware part of the memory can be read and/or written in the debug mode. The debug part of the memory may be read-protected and/or write-protected in at least one other mode of the intraoral scanning device, such as in the default mode and/or the customization mode. In debug mode, the handheld intraoral scanning device may be configured to transmit debug data that relates to the performance of the handheld intraoral scanning device, such as a temperature within the handheld intraoral scanning device during a scanning, the performance of the light projector and the image sensor of the handheld intraoral scanning device. Furthermore, the debug data may relate to the performance of the wireless interface during scanning and when no scanning is being performed.

The firmware data may include updates to the handheld intraoral scanning device that improves the functionality and features of the device.

The fourth mode may be an upgrade mode. An upgrade mode may be characterized in that an upgrade part of memory can be read and/or written in the upgrade mode. An upgrade mode may be characterized in that a firmware part of the memory is write-protected. The upgrade part of the memory may comprise intraoral scanning device data, such as improved features, new features relating to an operating software system, a FPGA or other electronic/digital hardware of the intraoral scanning device, such as a scanner throttle, a focus lens motor, a light projector(s), and/or image sensor.

The default mode may be a boot mode. A boot mode may be characterized in that the intraoral scanning device may be operated according to operating parameters set during booting and/or in response to user input via the user interface. The user input may include entering a scan mode, stop the scan mode, entering a command mode where the intraoral scanning device functions as a pointer in a software application, i.e., when moving the scanner then the cursor/pointer in the software moves correspondingly. The default mode may be characterized in that the firmware part (or at least a part thereof) and/or the customization part of the memory (or at least a part thereof) is write-protected and/or read-protected in the default mode. The default mode may be characterized in that the debug part of the memory (or at least a part thereof) is read-protected and/or write-protected in the default mode.

The intraoral scanning device may comprise a memory. The memory may be embedded in the processing unit and/or be employed in a memory unit connected to the processing unit. The memory may comprise a first memory part. The first memory part may be a firmware part of the memory. The firmware part of the memory may be configured to be accessed in the service mode e.g., to be written to and/or read from in the service mode. The firmware part of the memory may additionally be configured to be accessed in the debug mode. The memory may comprise a second memory part. The second memory part may be a customization part of the memory. The customization part of the memory may be configured to be accessed in the customization mode e.g., to be written to and/or read from in the customization mode. The customization part of the memory may additionally be configured to be accessed in the service mode and/or the debug mode. The memory may comprise a third memory part. The third memory part may be a debug part of the memory. The debug part of the memory may be configured to be accessed in the debug mode e.g., to be written to or read from in the debug mode. The memory may comprise a fourth memory part, The fourth memory part may be an upgrade part of the memory. The upgrade part of the memory may be configured to be accessed in the upgrade mode, e.g., to be written to or read from in the upgrade mode.

The intraoral scanning device may comprise a wireless interface configured to enable wireless communication between the intraoral scanning device and another device. The wireless interface may comprise a wireless transceiver, e.g., configured for wireless communication at frequencies in the range from 2.4 to 2.5 GHZ, 2.4 GHz to 5 GHZ, about 2.45 GHz or about 5 GHz. The wireless transceiver may be a Bluetooth transceiver, a Bluetooth Low Energy transceiver, or a Wireless Fidelity (WIFI) transceiver. The wireless interface may form a connection to one or more other devices such as a computer, and/or a scan computer, and/or a tablet and/or a smart phone.

The processing unit/intraoral scanning device may be configured to receive a mode request via the wireless interface. The mode request may comprise a mode identifier indicative of the requested mode. The mode request may be a service mode request, e.g., the mode identifier is indicative of a first/service mode. The mode request may be a customization mode request, e.g., the mode identifier is indicative of a second/customization mode. The mode request may be a debug mode request, e.g., the mode identifier is indicative of a third/debug mode. The mode request may be an upgrade mode request, e.g., the mode identifier is indicative of a fourth/upgrade mode. Accordingly, the mode request may be one of a service mode requests, a customization mode request, an upgrade mode request and a debug mode request.

The intraoral scanning device may be placed into the requested mode if the intraoral scanning device is not placed in a scanning session. The intraoral scanning device is scanning in a scanning session when being placed in the scanning session.

The placing of the intraoral scanning device into the requested mode may be scheduled for a specific time on a day when the intraoral scanning device will not be used. The scheduling may be determined by the processing unit based on historical usage time of the intraoral scanning device and a machine learning model. The machine learning model receives timestamps from a clock in the intraoral scanning device and input information about when the intraoral scanning device is being used in a scanning session. The machine learning model includes a training data set which includes historical usage time of the intraoral scanning device being in the scanning session. Based on the machine learning model and a timestamp defining the time of the day the processing unit will know when to be set into a requested mode if receiving a mode request. The advantage of the scheduling is that a valid authenticated mode request will not interfere the work of the dentist with the intraoral scanning device. Furthermore, when being placed into the customization mode, the intraoral scanning device can be programmed to do time consuming updates within specific time-period(s). For example, an update which last more than 30 mins will automatically be planned to be performed in a time-period of more than 30 mins where the intraoral scanning device will not be used, such as outside the working hours or during a break of the dentist/clinic.

The processing unit may be configured to place the intraoral scanning device into the requested mode if authentication of the mode request succeeds and if a timestamp is within a time-period. The timestamp is generated by a clock of the intraoral scanning device and received by the processing unit.

The processing unit may include a machine learning model that includes a training data set which includes historical data the relates to usage time of the intraoral scanning device being in a scanning session, and wherein the machine learning model receives a timestamp from a clock in the intraoral scanning device and input information about when the intraoral scanning device is being used in a scanning session, and the processing unit may then be configured to place the intraoral scanning device into the requested mode if authentication of the mode request succeeds and if the machine learning model outputs a trigger that allows the intraoral scanning device to be placed into the mode.

The mode request may comprise a sender identifier indicative of the mode request sender. The mode request may comprise a certificate, such as a digital signature, for certifying the mode request sender. This allows for direct authentication of the mode request. The mode request may comprise a session identifier, e.g., an encrypted session identifier.

The intraoral scanning device may be paired with a sender of the mode request prior to receipt of the mode request. In the pairing, the intraoral scanning device and the sending/client device may have exchanged one or more of intraoral scanning device identifier, sender identifier, session identifier, etc.

The processing unit/intraoral scanning device is configured to authenticate the mode request and to place the intraoral scanning device into the requested mode if authentication of the mode request succeeds. The processing unit may be configured to place the intraoral device into a mode different from the requested mode, such as the default mode, if authentication of the mode request fails.

The intraoral scanning device disclosed herein has the advantage of verifying integrity of received mode requests and/or senders thereof, detecting any alteration and disregard altered mode requested. The intraoral scanning device disclosed herein may advantageously allow access to specific parts of the memory only with authenticated parties, such as an authenticated scan computer, an authenticated computer, an authenticated accessory device, an authenticated external device and/or an authenticated server.

The processing unit may be configured to authenticate the mode request by authenticating the sender of the mode request.

The processing unit/intraoral scanning device may be configured to authenticate the mode request by verifying integrity of a digital signature of the mode request. The processing unit may be configured to authenticate the mode request by verifying integrity of the mode request. The mode request may comprise a message authentication code (MAC). To verify integrity of the mode request may comprise to verify the message authentication code, e.g., with a session identifier stored in the intraoral scanning device. The mode request may comprise a digital signature or certificate. To verify integrity of the mode request may comprise verifying the digital signature or certificate.

The processing unit/intraoral scanning device may be configured to send a mode response. For example, to place the intraoral scanning device into the requested mode if authentication of the mode request succeeds may comprise sending a mode response. The processing unit/intraoral scanning device may be configured to generate and/or send a mode response in response to the mode request. The processing unit may be configured to obtain and/or store a session identifier (may also be denoted session key) and include the session identifier and/or an encrypted version thereof in the mode response. To obtain the session identifier may comprise to generate the session identifier, e.g., as a random or pseudo-random number. Thus, the intraoral scanning device and/or the processing unit may comprise a number generator, e.g., configured to generate a random or pseudo-random number as a session identifier. By using a unique session identifier or session identifier from a large number of available session identifiers, the processing power requirements in the intraoral scanning device may be reduced. Further, simple encryption is facilitated, and replay-attacks are prevented.

The processing unit may be configured to encrypt the session identifier, optionally based on an intraoral scanning device key. The session identifier may be a session key in the form of a symmetric key. A symmetric session key may provide a lightweight processing of the security algorithms on the processing unit, such as lightweight encryption, lightweight decryption, lightweight integrity protection, etc. The intraoral scanning device key may be a symmetric key or a public key of a private-public key pair. The intraoral scanning device key may be stored in a permanent memory of the intraoral scanning device, e.g., during manufacture or during a customization session.

The mode response may comprise the encrypted session key. The session response may comprise an intraoral scanning device identifier and/or the session key. Thus, the processing unit may be configured to send an intraoral scanning device identifier and/or the session key in the mode response. A mode response comprising an intraoral scanning device identifier may enable the sender of the mode request to obtain the intraoral scanning device key, either from a database or by requesting the intraoral scanning device key from the manufacturer, which in turn enables the sender of the mode request to decrypt an encrypted session identifier/key and use the session identifier when sending data to the intraoral scanning device.

The mode request may be received in a session. The processing unit/intraoral scanning device may be configured to terminate the session if authentication of the mode request fails.

The mode request may comprise a signature, and to authenticate the mode request may comprise to verify the signature of the mode request.

The processing unit may be configured to obtain, e.g., generate a session identifier, e.g. upon receipt of the mode request or when the intraoral scanning device is in a service mode, a customization mode, or a debug mode. The processing unit may be configured to encrypt the session identifier, e.g., with an intraoral scanning device key. The processing unit may be configured to transmit the session identifier or the encrypted session identifier via the wireless interface, e.g., as a part of the mode response or a session setup message. The processing unit may be configured to store the session identifier in the intraoral scanning device.

The processing unit may be configured to receive data via the wireless interface, e.g., when the intraoral scanning device is in a mode, e.g. the service mode, the customization mode and/or the debug mode. The processing unit may be configured to authenticate the received data, e.g., when the intraoral scanning device is in one or more modes, e.g. the service mode, the customization mode and/or the debug mode. The processing unit may be configured to store intraoral scanning device data in a part of the memory based on the received data if authentication of the data succeeds. For example, when the intraoral scanning device is in a service mode, the processing unit may store intraoral scan data, such as e.g., firmware, based on the received data in the firmware part of the memory. In an exemplary intraoral scanning device, the processing unit may, when the intraoral scanning device is in a customization mode, store intraoral scan data (such as customization data) based on the received data in the customization part of the memory. In an exemplary intraoral scanning device, the processing unit may, when the intraoral scanning device is in a debug mode, store intraoral scanning device data (debug data) based on the received data in the debug part of the memory.

The processing unit may be configured to authenticate the received data by verifying integrity of the received data. Verifying integrity of the received data may be based on the session identifier stored in the intraoral scanning device. The received data may comprise a message authentication code. To verify integrity of the received data may comprise to verify the message authentication code, e.g., with the stored session identifier. The received data may comprise a digital signature. To verify integrity of the received data may comprise verifying the digital signature.

The data may comprise a session identifier, and to authenticate the data may comprise to compare the session identifier of received data with the session identifier stored in the intraoral scanning device.

The data may be received in a session. The processing unit may be configured to terminate the session if authentication of the received data fails, e.g., the processing unit may be configured to terminate the session if integrity of the received data is corrupted, i.e. verification of the integrity fails. The processing unit may be configured to place the intraoral scanning device in another mode, such as the default mode, if authentication of the received data fails.

The intraoral scanning device/processing unit may be configured to receive a mode exit request and to place the intraoral scanning device in another mode, such as the default mode, e.g., if an authentication of the mode exit request succeeds. For example, a client device may send a mode exit request when customization or transfer of firmware is done.

The disclosed method provides secure configuration and/or update of an intraoral scanning device. The method may comprise placing the intraoral scanning device into a default mode if authentication of the mode request fails. The method may comprise determining if operation in default mode fails and switching to service mode if operating the intraoral scanning device in default mode fails.

In the method, authenticating the mode request may comprise authenticating the sender of the mode request.

In the method, the mode request may comprise a digital signature, and authenticating the mode request may comprise verifying the digital signature.

In the method, authenticating the mode request may comprise verifying integrity of the mode request.

The method may comprise receiving data via the wireless interface, e.g., when the intraoral scanning device is in one or more modes, e.g. the service mode, the customization mode, the upgrade mode and/or the debug mode. The method may comprise authenticating the received data, e.g., when the intraoral scanning device is in one or more modes, e.g. the service mode, the customization mode, the upgrade mode and/or the debug mode. The method may comprise storing intraoral scanning device data in a part of the memory based on the received data if authentication of the data succeeds. For example, when the intraoral scanning device is in a service mode, the method may comprise storing intraoral scanning device data (firmware) based on the received data in the firmware part of the memory. In an exemplary method, the method may, when the intraoral scanning device is in a customization mode, comprise storing intraoral scanning device data (such as customization data, scanning settings) based on the received data in the customization part of the memory. In an exemplary method, the method may, when the intraoral scanning device is in a debug mode, comprise storing intraoral scanning device data (debug data) based on the received data in the debug part of the memory. In an exemplary method, the method may, when the intraoral scanning device is in an upgrade mode, comprise storing intraoral scanning device data (such as data including improved features, new features relating to an operating software system, a FPGA or other electronic/digital hardware of the intraoral scanning device) based on the received data in the debug part of the memory. The method may comprise placing the intraoral scanning device in another mode, such as the default mode, if authenticating the received data fails.

The processing unit may be configured to operate the intraoral scanning device in default mode, and switch to service mode if operating the intraoral scanning device in default mode fails.

Intraoral Scanning Device with Communication Protection and Related Method:

Furthermore, the present disclosure relates to an intraoral scanning device with communication protection and related method, and in particular to an intraoral scanning device for communicating securely with accessory devices/systems and related method.

According to the aspects, an intraoral scanning device for acquiring intraoral scan data from a three-dimensional dental object during a scanning session is disclosed. The intraoral scanning device may comprise a processing unit configured to process intraoral scan data of a patient and provide 2D image data and/or 3D image data: a memory; and a wireless interface configured for transmitting the 2D image data and/or the 3D image data. Furthermore, the processing unit may be configured to receive a session request for a session via the wireless interface: obtain and store a session key: sign the session key by an intraoral scanning device key, and wherein the intraoral scanning device key may be stored in a permanent memory of the intraoral scanning device. The processing unit may be configured to send a session response that may comprise the signed session key and to receive session data in the session via the wireless interface.

According to the aspects, an intraoral scanning device for acquiring intraoral scan data from a three-dimensional dental object during a scanning session is disclosed. The intraoral scanning device may comprise a processing unit configured to process intraoral scan data of a patient and provide 2D image data and/or 3D image data: a memory; and a wireless interface configured for transmitting the 2D image data and/or the 3D image data. Furthermore, the processing unit may be configured to receive a session request for a session via the wireless interface: obtain and store a session key: encrypt the session key based on an intraoral scanning device key, and wherein the intraoral scanning device key may be stored in a permanent memory of the intraoral scanning device. The processing unit may be configured to send a session response that may comprise the encrypted session key and to receive session data in the session via the wireless interface.

According to the aspects, an intraoral scanning device for acquiring intraoral scan data from a three-dimensional dental object during a scan session is disclosed. The intraoral scanning device may comprise a processing unit configured to process intraoral scan data of a patient and provide 2D image data and/or 3D image data: a memory; and a wireless interface configured for transmitting the 2D image data and/or the 3D image data. Furthermore, the wireless interface may be configured to transmit a session request to the processing unit. Furthermore, the processing unit may be configured to obtain and store a session key and to encrypt the session key based on an intraoral scanning device key, that may be stored in a permanent memory of the intraoral scanning device. The processing unit may be further configured to send a session response that may comprise the encrypted session key and receive session data in the session via the wireless interface.

According to the aspects, a method for communication with an intraoral scanning device for acquiring intraoral scan data from a three-dimensional dental object during a scanning session is disclosed. The method may comprise a processing unit configured to process intraoral scan data of a patient and provide 2D image data and/or 3D image data, a memory unit, and a wireless interface. Furthermore, the method may comprise receiving a session request for a session via the wireless interface, obtaining and storing a session key, and signing the session key by an intraoral scanning device key, wherein the intraoral scanning device key may be stored in a permanent memory of the intraoral scanning device. Furthermore, the method may comprise sending a session response comprising the signed session key and receiving session data in the session via the wireless interface.

According to the aspects, a method for communication with an intraoral scanning device for acquiring intraoral scan data from a three-dimensional dental object during a scanning session is disclosed. The method may comprise a processing unit configured to process intraoral scan data of a patient and provide 2D image data and/or 3D image data, a memory unit, and a wireless interface. Furthermore, the method may comprise receiving a session request for a session via the wireless interface, obtaining and storing a session key, and encrypting the session key based on an intraoral scanning device key, wherein the intraoral scanning device key may be stored in a permanent memory of the intraoral scanning device. Furthermore, the method may comprise sending a session response comprising the encrypted session key and receiving session data in the session via the wireless interface.

According to the aspects, a method for communication with an intraoral scanning device for acquiring intraoral scan data from a three-dimensional dental object during a scanning session is disclosed. The method may comprise a processing unit configured to process intraoral scan data of a patient and provide 2D image data and/or 3D image data, a memory unit, and a wireless interface. Furthermore, the method may comprise transmitting a session request to the processing unit, obtaining, and storing a session key and encrypting the session key based on an intraoral scanning device key, wherein the intraoral scanning device key may be stored in a permanent memory of the intraoral scanning device. Furthermore, the method may comprise sending a session response comprising the encrypted session key and receiving session data in the session via the wireless interface.

An intraoral scanning device is in a scanning session when it is being used intentionally, such as for scanning of an oral cavity of a patient.

The session key may be signed before being encrypted based on the intraoral scanning device key, and where the session response includes the encrypted session key. The processing unit may be configured to verify integrity of the session data. The encryption of the session key provides an additional layer of security when distributing session key in between the intraoral scanning device and an external device(s).

The intraoral scanning device comprises a processing unit. The processing unit may be configured to process intraoral scan data of a patient and provide 2D image data and/or 3D image data. The 2D image data and/or 3D image data may include information about the anatomy of the oral cavity of the patient, such as teeth, gingival, bone level, and/or information about diagnostic indicators such as caries, bone loss, gingivitis, gingiva recession, periodontitis, bone loss, cracks, and occlusion.

The 2D image data and/or the 3D image data may be image data configured to be visualizable on a display in a 2D or a 3D manner, respectively.

The session data may comprise customization data, and the customization data may include, for example, settings of a color image sensor of an intraoral scanning device. An intraoral scanning device may include a color image sensor, such as an RGB image sensor where the customization data may include information about different color areas to be deactivated and/or activated during at least a scanning session. Thus, the customization data may relate to which color areas of the RBG image sensor should be activated or deactivated during a scanning session. An intraoral scanning device may include a monochromatic image sensor and colored light emitting diodes, and in this example, the customization data may include information about which of the different colored light emitting diodes should be deactivated and/or activated during a scanning session. Thus, the customization data may include information that relates to which colored light emitting diodes should be activated or deactivated during a scanning session. A colored light emitting diode may be configured to emit light with a color, such as blue, red, green etc. In another example, the intraoral scanning device could include one or more near-infrared light emitting diodes which also can be set to be activated and/or deactivated during a scanning session by the customization data. The customization data may include setting data, such as power management settings, configuration of a user interface of the intraoral scanning device and/or settings of an optical unit of the intraoral scanning device

The optical unit may include one or more light projectors, one or more optical components, and one or more image sensors.

The intraoral scanning device may comprise a processing unit, a memory unit, and a wireless interface. The wireless interface may comprise a wireless transceiver, e.g., configured for wireless communication at frequencies in the range from 2.4 to 2.5 GHZ, 2.4 GHz to 5 GHz, or about 2.4 GHz or about 5 GHz.

The processing unit may be configured to receive a session request for a session via the wireless interface. The processing unit may be configured to verify the session request, such as authenticate the sender of the session request, e.g., a client device or a server device. It may be envisaged that the intraoral scanning device and the sender of the session request have pre-established authenticated connection which thus permits the session request to be authenticated by the intraoral scanning device. The session request may comprise a digital signature enabling authentication of the session request.

In one or more exemplary intraoral scanning devices, the session request comprises a digital signature. The processing unit may be configured to verify integrity of the session request, e.g., by verifying the digital signature. For example, a verifier of the processing unit may be configured to verify the digital signature. The processing unit verifies the digital signature using a signature verification function and a public key of a sender that has generated the digital signature and included the digital signature in the session request. If the intraoral scanning device/processing unit determines that the digital signature is not successfully verified using the alleged public key of a sender, the intraoral scanning device disregards the session request and terminates the session. This may provide the advantage that the intraoral scanning device rejects session requests from unauthenticated parties, thereby reducing the risk of or limit the effects of a battery exhaustion attack.

The intraoral scanning device may be paired with a sender of the session request prior to receipt of the session request. In the pairing, the intraoral scanning device and the sending/client device may have exchanged one or more of intraoral scanning device identifier, sender identifier, session key/identifier, etc.

The processing unit is configured to obtain and/or store a session key; and encrypt the session key, optionally based on an intraoral scanning device key. The session key may be a symmetric key. A symmetric session key may provide a lightweight processing of the security algorithms on the processing unit, such as lightweight encryption, lightweight decryption, lightweight integrity protection, etc.

The processing unit is configured to obtain the session key, and to obtain the session key may comprise to generate the session key, e.g., as a random or pseudo-random number. Thus, the intraoral scanning device and/or the processing unit may comprise a number generator, e.g., configured to generate a random or pseudo-random number. By using a unique session key or session key from a large number of available session keys, the processing power requirements in the intraoral scanning device may be reduced. Further, simple encryption is facilitated, and replay-attacks are prevented.

The intraoral scanning device key may be a symmetric key or a public key of a private-public key pair. The intraoral scanning device key may be stored in a permanent memory of the intraoral scanning device, e.g., during manufacture or during a customization session.

The processing unit is configured to send a session response in response to the session request. The session response may comprise the encrypted session key. The session response may comprise an intraoral scanning device identifier and/or the session key. Thus, the processing unit may be configured to send an intraoral scanning device identifier and/or the session key in the session response. A session response comprising an intraoral scanning device identifier may enable the sender of the session request to obtain the intraoral scanning device key, either from a database or by requesting the intraoral scanning device key from the manufacturer, which in turn enables the sender of the session request to decrypt the session key and use the session key when sending session data to the intraoral scanning device.

The intraoral scanning device disclosed herein has the advantage of verifying integrity of received data, detecting any alteration and disregard altered data. The intraoral scanning device disclosed herein has the advantage to open a session only with authenticated parties, such as an authenticated customization device, an authenticated accessory device, an authenticated external device and/or an authenticated server.

The processing unit is configured to receive session data in the session via the wireless interface. The processing unit may be configured to verify integrity of the session data. The session data may comprise a message authentication code. To verify integrity of the session data may comprise to verify the message authentication code, e.g., with the stored session key. The session data may comprise a digital signature. To verify integrity of the session data may comprise verifying the digital signature.

The processing unit may be configured to terminate the session if integrity of the session data is corrupted, i.e., verification of the integrity fails.

The processing unit may be configured to decrypt the session data with the session key. The processing unit may be configured to store at least part of decrypted session data in the memory unit. The processing unit may be configured to terminate the session if decryption of the session data fails. The session data may comprise customization data, intraoral scanning device operating parameters, and/or firmware data.

The intraoral scanning device operating parameters may corresponds to settings of the handheld intraoral scanning device that involves settings of the image sensor, light projector, the wireless interface, a scan sequence of the handheld intraoral scanning device. Etc. The scan sequence corresponds to a scanning of a patient's jaws with the handheld intraoral scanning device, while in real-time the handheld intraoral scanning device is configured to determine and transmit the 3D image data based on the intraoral scan data acquired by the image sensor of the handheld intraoral scanning device during the scan sequence.

Furthermore, the intraoral scanning device operating parameters relates to power management settings, configuration of a user interface of the intraoral scanning device and/or settings of an optical unit of the intraoral scanning device.

The handheld intraoral scanning device may include a user interface which may include at least a touch sensor, at least a touch button, at least a light emitting diode, a haptic sensor, and/or an accelerometer. The handheld intraoral scanning device may include a motion sensor which is configured to sense the motion of the handheld intraoral scanning device. The handheld intraoral scanning device is configured to communicate wirelessly with an external device that is connected to a display. A cursor on the display may be moved around based on motion signals provided by the motion sensor to the external device. The user is able to navigate the cursor on the display by moving the handheld intraoral scanning device. The session data may include settings update that relates to the motion sensor of the handheld intraoral scanning device, and the customization data may include settings for customizing a user interface of the handheld intraoral scanning device which may involve a graphical setup of a graphical user interface on the display. For example, when the handheld intraoral scanning device connects to the external device, the handheld intraoral scanning device forwards a customization package to the external device via the wireless interface, and the external device is then configured to change the graphical setup based on the customization package. The customization package may be updated by the customization data provided by the session data.

The firmware data may include updates to the handheld intraoral scanning device that improves the functionality and features of the device.

The processing unit may be configured to process intraoral scan data of a patient and provide 2D image data and/or 3D image data according to the received session data. Thus, a sender of the session request may control operation of the intraoral scanning device, either by sending customization data, intraoral scanning device operating parameters, and/or firmware data. The disclosed intraoral scanning device and method prevents unauthorized access or control of an intraoral scanning device.

The session data may be relevant for providing image data with improved quality or with more features. In this example, the session data may be relevant for the optical unit or with firmware updates that improves the processing of the intraoral scan data into the image data.

The intraoral scanning device being able to send a session response may be scheduled for a specific time on a day where the intraoral scanning device will not be used. The scheduling may be determined by the processing unit based on historical usage time of the intraoral scanning device and a machine learning model. The machine learning model receives timestamps from a clock in the intraoral scanning device and input information about when the intraoral scanning device is being used in a scanning session. The machine learning model includes a training data set which includes historical usage time of the intraoral scanning device being in the scanning session. Based on the machine learning model and a timestamp defining the time of the day the processing unit will know when to be set into a requested mode if receiving a mode request. The advantage of the scheduling is that a session request will not interfere the work of the dentist with the intraoral scanning device. Furthermore, the processing unit may be configured to do time consuming updates within specific time-period(s). For example, an update which last more than 30 mins will automatically be planned to be performed in a time-period of more than 30 mins where the intraoral scanning device will not be used, such as outside the working hours or during a break of the dentist/clinic. In other words, the processing unit is configured to plan an update based on an estimated time for installing the update to a firmware of the memory unit.

The processing unit may be configured to send a session response if a timestamp is within a time-period. The timestamp is generated by a clock of the intraoral scanning device and received by the processing unit.

The processing unit may include a machine learning model that includes a training data set which includes historical data the relates to usage time of the intraoral scanning device being in a scanning session, and wherein the machine learning model receives a timestamp from a clock in the intraoral scanning device and input information about when the intraoral scanning device is being used in a scanning session, and the processing unit may then be configured to send a session response if the machine learning model outputs a trigger that allows the processing unit to send the session response.

The session data may relate to optical data that are relevant for the optical unit, for processing intraoral scan data, and for providing image data. The optical data may include settings of the light projector(s), the image sensor(s), the motor for the focus lens or firmware/settings for providing trigonometry calculation that includes the emitted light from the light projector(s) and the reflected light received by the image sensor(s). Furthermore, the optical data may include modifications, updates, or a new computer-implemented method for processing the intraoral scanning data into 2D image data and/or 3D image data.

The session request corresponds to an optical session request that are relevant for transmitting session data that relates to optical data.

As used herein, the term “intraoral scanning device” refers to a device configured to conduct a scan inside the oral cavity of a patient, or a part thereof, or parts thereof, such as a tooth, teeth, gingiva, etc., or to obtain a 2D image data and/or 3D image data of the oral cavity of a patient or parts thereof, such as a tooth, teeth and/or gingiva, etc. the intraoral scanning device may be an intraoral scanner that is fully or partly inserted in the oral cavity of a patient, such as a wireless intraoral handheld scanner.

### An Intraoral Scanning Device and Method of Intraoral Scanning Device Communication:

The present disclosure pertains to the field of intraoral scanning devices, and in particular to intraoral scanning device security. Intraoral scanning device and method for secure intraoral scanning device communication is disclosed.

An even further aspect of the present disclosure is to provide the intraoral scanning device the capability of securing access thereto from unauthenticated parties and securing its communication against modification attacks and replay attacks while minimizing computational overhead and power consumption of the intraoral scanning device. Furthermore, the present disclosure provides a scalable security architecture.

According to the aspect, an intraoral scanning device configured to acquire intraoral scan data from a three-dimensional dental object during a scanning session is disclosed. The intraoral scanning device may comprise a processing unit configured to process intraoral scan data of a patient and provide 2D image data and/or 3D image data, a wireless interface configured to transmit the 2D image data and/or the 3D image data, and a memory. The processing unit may be configured to receive a linking request for a session via the wireless interface, obtain a session identifier, transmit, via the wireless interface, a linking response comprising an intraoral scanning device identifier and the session identifier. Furthermore, the processing unit may be configured to receive, via the wireless interface, an authentication message comprising an authentication key identifier and client device data, select an intraoral scanning device key from a plurality of intraoral scanning device keys in the memory unit based on the authentication key identifier, verify the client device data based on the selected intraoral scanning device key, and terminate the session if the verification fails.

According to the aspect, a method for configuration of an intraoral scanning device that may comprise a processing unit configured to process intraoral scan data of a patient and provide 2D image data and/or 3D image data, a memory unit and a wireless interface configured for transmitting the 2D image and/or the 3D image. The method may comprise receiving a linking request for a session via the wireless interface, obtaining a session identifier, transmitting, via the wireless interface, a linking response comprising an intraoral scanning device identifier and the session identifier, receiving, via the wireless interface, an authentication message comprising an authentication key identifier and client device data, selecting an intraoral scanning device key from a plurality of intraoral scanning device keys based on the authentication key identifier, verifying the client device data based on the selected intraoral scanning device key; and terminating the session if verification fails.

According to the aspect, an intraoral scanning device for acquiring intraoral scan data from a three-dimensional dental object during a scanning session is disclosed. The intraoral scanning device may comprise a processing unit configured to process intraoral scan data of a patient and provide 2D image data and/or 3D image data, a wireless interface configured for transmitting the 2D image data and/or the 3D image data, and a memory. The processing unit may be configured to receive a connection request for a session via the wireless interface, obtain a session identifier, transmit, via the wireless interface, a connection response comprising an intraoral scanning device identifier and the session identifier. Furthermore, the processing unit may be configured to receive, via the wireless interface, an authentication message comprising an authentication key identifier and client device data, select an intraoral scanning device key from a plurality of intraoral scanning device keys in the memory unit based on the authentication key identifier, verify the client device data based on the selected intraoral scanning device key, and terminate the session if the verification fails.

The intraoral scanning device is a handheld scanning device for scanning inside an oral cavity of a patient. The intraoral scanning device differs from other type of teeth scanning device in that the intraoral scanning device is a handheld scanning device which can easily be handled by one hand by a user, and which has now wired connection to any external device during scanning of an inside of an oral cavity of a patient. Therefore, the only attack which an intraoral scanning device may experience is via the wireless interface.

The method and the intraoral scanning device as disclosed provide secure configuration of the intraoral scanning device, such as secure access to the memory of the intraoral scanning device. It is an advantage of the present disclosure that the intraoral scanning device can only be configured or updated by authorized parties. The disclosed intraoral thus has the advantage of detecting and preventing any modification by unauthorized parties. The intraoral scanning device disclosed herein is advantageously protected against attacks such as spoofing attacks, man-in-the-middle attacks, and/or replay-attacks.

The intraoral scanning device is the key element in providing the needed level of security in wireless communication in a service infrastructure which at least includes the intraoral scanning device and a scan computer or a dental software on a computer. It would not be possible for a third party to attack the wireless communication as this person needs to have the intraoral scanning device physically in its hand. It would not even be enough to have access to the scan computer or the dental software.

The method as disclosed herein provides a secure configuration and/or update of an intraoral scanning device.

The present disclosure provides improved security of an intraoral scanning device. Security comprises assessing threats, vulnerabilities and attacks and developing appropriate safeguards and countermeasures to protect against threats and attacks.

The intraoral scanning device comprises a processing unit. The processing unit may be configured to process intraoral scan data of a patient and provide 2D image data and/or 3D image data. The 2D image data and/or 3D image data may include information about the anatomy of the oral cavity of the patient, such as teeth, gingival, bone level, and/or information about diagnostic indicators such as caries, bone loss, gingivitis, gingiva recession, periodontitis, bone loss, cracks, and occlusion.

The 2D image data and/or the 3D image data may be image data configured to be visualizable on a display in a 2D or a 3D manner, respectively.

As used herein, the term “certificate” refers to a data structure that enables verification of its origin and content, such as verifying the legitimacy and/or authenticity of its origin and content. The certificate may be configured to provide a content that is associated to a holder of the certificate by an issuer of the certificate. The certificate comprises a digital signature, so that a recipient of the certificate is able to verify or authenticate the certificate content and origin. The certificate may comprise one or more identifiers and/or keying material, such as one or more cryptographic keys (e.g., an intraoral scanning device key) enabling secure communication in an intraoral scanning device system. The certificate permits thus to achieve authentication of origin and content, non-repudiation, and/or integrity protection. The certificate may further comprise a validity period, one or more algorithm parameters, and/or an issuer. A certificate may comprise a digital certificate, a public key certificate, an attribute certificate, and/or an authorization certificate.

As used herein, the term “key” refers to a cryptographic key, i.e., a piece of data, (e.g. a string, a parameter) that determines a functional output of a cryptographic algorithm. For example, during encryption, the key allows a transformation of a plaintext into a cipher-text and vice versa during decryption. The key may also be used to verify a digital signature and/or a message authentication code, MAC. A key is so called a symmetric key when the same key is used for both encryption and decryption. In asymmetric cryptography or public key cryptography, a keying material is a key pair, so called a private-public key pair comprising a public key and a private key. In an asymmetric or public key cryptosystem (such as Rivest Shamir Adelman, RSA, cryptosystem, and elliptic curve cryptography, ECC), the public key is used for encryption and/or signature verification while the private key is used for decryption and/or signature generation. The intraoral scanning device key may be keying material allowing deriving one or more symmetric keys, such as a session key and/or a certificate key for intraoral scanning device communication. The intraoral scanning device key may be stored in a memory unit of the intraoral scanning device, e.g., during manufacture. The intraoral scanning device key may comprise keying material that is used to derive a symmetric key. The intraoral scanning device key comprises for example an Advanced Encryption Standard, AES, key, such as an AES-128 bits key.

As used herein the term “identifier” refers to a piece of data that is used for identifying, such as for categorizing, and/or uniquely identifying. The identifier may be in a form of a word, a number, a letter, a symbol, a list, an array, or any combination thereof. For example, the identifier as a number may be in the form of an integer, such as unsigned integer, uint, with a length of e.g., 8 bits, 16 bits, 32 bits, etc., such as an array of unsigned integers.

The term “client device” as used herein refers to a device that is able to communicate with the intraoral scanning device. The client device may refer to a computing device acting as a client. The client device may comprise a customization device, a relay, a tablet, a personal computer, an application running on a personal computer or tablet, and/or USB dongle plugged into a personal computer.

The present disclosure relates to an intraoral scanning device. The intraoral scanning device may comprise a processing unit configured to process intraoral scan data of a patient and provide 2D image data and/or 3D image data, a memory unit, and a wireless interface. The memory unit may include removable and non-removable data storage units including, but not limited to, Read Only Memory (ROM), Random Access Memory (RAM), etc. The memory unit may have an intraoral scanning device certificate stored thereon. The memory unit may have the intraoral scanning device certificate stored at a memory address of the memory unit, and/or in memory cells of the memory unit, such as in designated memory cells and/or at designated addresses. The wireless interface may comprise a wireless transceiver, e.g., configured for wireless communication at frequencies in the range from 2.4 to 2.5 GHZ, 2.4 GHz to 5 GHZ, about 2.45 GHz or about 5 GHz. In one or more exemplary intraoral scanning devices, the wireless interface is configured for communication, such as wireless communication, with a client device or an intraoral scanning device, respectively comprising a wireless transceiver configured to receive and/or transmit data. The processing unit may be configured to receive a linking request for a session via the wireless interface; and to obtain a session identifier, e.g., in response to the linking request. The wireless interface may be configured to receive the linking request for a session from a client device. The processing unit may be configured to obtain a session identifier, such as by generating a random or pseudo-random number. The processing unit may be configured to store the session identifier in the memory unit. The memory unit may be configured to store the session identifier at a memory address of the memory unit, and/or in memory cells of the memory unit, such as in designated memory cells and/or at designated addresses. The linking request may comprise an authentication key identifier and/or an authentication type identifier, in order to permit the intraoral scanning device to perform authentication of the linking request and the client device sending the linking request at this early stage. This may provide a level of access control.

The processing unit may be configured to transmit via the wireless interface a linking response comprising an intraoral scanning device identifier and the session identifier. The processing unit may be configured to generate a linking response by including the session identifier and the intraoral scanning device identifier in the linking response. The intraoral scanning device identifier may refer to a unique identifier of the intraoral scanning device. The intraoral scanning device identifier may be included in the intraoral scanning device certificate. The wireless interface may be configured to transmit the linking response to e.g., the client device.

The processing unit may be configured to receive, via the wireless interface, an authentication message comprising an authentication key identifier and client device data. For example, the wireless interface may be configured to receive the authentication message from the client device. For example, the intraoral scanning device receives the authentication message from the client device in order to establish a communication session. The client device data may comprise a client device certificate (encrypted or unencrypted), customization data, intraoral scanning device operating parameters, and/or firmware data. For example, the authentication message may comprise an authentication key identifier in plain text. The authentication key identifier is indicative of an intraoral scanning device key, an intraoral scanning device key stored in the memory unit of the intraoral scanning device, for example as part of the intraoral scanning device certificate.

The processing unit may be configured to select an intraoral scanning device key from a plurality of intraoral scanning device keys in the memory unit, based on the authentication key identifier and optionally other identifiers. When the authentication key identifier is acceptable by the intraoral scanning device based on an intraoral scanning device key identifier held by the intraoral scanning device, the processing unit may be configured to select an intraoral scanning device key that the authentication key identifier indicates and to use the selected intraoral scanning device key as keying material in securing the session. The processing unit may be configured to select an intraoral scanning device key from a plurality of intraoral scanning device keys in the memory unit based on the authentication key identifier and an authentication type identifier.

The authentication type identifier may be received in plaintext by the intraoral scanning device, and/or as client device type identifier in the client device certificate (encrypted or decrypted). For example, the processing unit may be configured to select an intraoral scanning device key which the authentication key identifier and the authentication type identifier indicate.

The processing unit may be configured to verify the client device data, based on the selected intraoral scanning device key; and to terminate the session if verification fails. To verify the client device data may be based on an intraoral scanning device certificate or at least parts thereof. To verify the client device data based on the selected intraoral scanning device key may comprise verifying the integrity of the client device data based on the selected intraoral scanning device key, such as verifying a MAC and/or a digital signature comprised in the client device data. To verify the client device data based on the selected intraoral scanning device key may comprise decrypting the client device data, e.g., a client device certificate, using the selected intraoral scanning device key (as keying material to derive a decryption key or as a decryption key), when the client device data is received encrypted. To verify the client device data based on the selected intraoral scanning device key may comprise verifying the client device data, e.g., decrypted client device certificate, by comparing the received client device data with data stored in the memory unit. The client device data may comprise a client device certificate (such as an encrypted client device certificate), an authentication key identifier, and/or an authentication type identifier. The client device may be assigned a client device certificate. The client device certificate refers to a certificate generated and assigned to the client device by e.g., a device manufacturing the client device.

The client device certificate may comprise a certificate type identifier. The certificate type identifier may indicate a type of the certificate amongst a variety of certificate types, such as an intraoral scanning device family certificate type, an intraoral scanning device certificate type, a firmware certificate type, a research and development certificate type, client device certificate type. The certificate type identifier may be used by the intraoral scanning device to identify what type of certificate it receives, stores, and/or retrieves. The client device certificate may comprise a version identifier indicative of a data format version of the certificate. The intraoral scanning device may be configured to use the certificate type identifier and/or the version identifier to determine what type of data the certificate comprises, what type of data is comprised in a field of the certificate. For example, the intraoral scanning device determines based on the certificate type identifier and/or version identifier what field of the certificate comprises a digital signature and/or which public key is needed to verify the digital signature. It may be envisaged that there is a one-to-one mapping between the certificate type identifier and the public-private key pair.

The client device certificate may comprise a signing device identifier. The signing device identifier refers to a unique identifier identifying the device (such as a manufacturing device, e.g., an integrated circuit card, a smart card, a hardware security module) that has signed the client device certificate. The signing device identifier may for example comprise a medium access control, MAC, address of the signing device and/or a serial number. The signing device identifier optionally allows for example the intraoral scanning device to determine whether the signing device is e.g., black-listed or not, and thus to reject certificates signed by a signing device that is black-listed.

The client device certificate may comprise one or more hardware identifiers such as a first hardware identifier and/or a second hardware identifier. A hardware identifier may identify a piece of hardware comprised in the client device, such as a radio chip comprised in the client device or a digital signal processor of the client device. The hardware identifier may be stored in a register of the piece of hardware comprised in the intraoral scanning device during manufacturing of the piece of hardware. The hardware identifier may comprise a serial number, a medium access control, MAC, address, a chip identifier, or any combination thereof. The client device certificate may comprise a client device type identifier. A client device type identifier may be indicative of a type which the client device belongs to. The client device may be attributed a client device type corresponding to a model, category or type of client devices, such as a customization type, e.g., a computer product model, category or type configured for customizing the intraoral scanning device, a USB dongle product model, category or type configured for customizing the intraoral scanning device.

The client device certificate may comprise a client device identifier. The client device identifier refers to an identifier identifying a client device. The client device identifier may for example comprise a medium access control, MAC, address of the client device, and/or a serial number of the client device.

The client device certificate may comprise a client device key identifier. A client device key identifier may be indicative of the client device key used as keying material for securing a communication with an external party, such as with an intraoral scanning device. In one or more exemplary client device certificates, the client device certificate comprises a Bluetooth address or an IP address of the client device.

The client device certificate comprises a digital signature. The digital signature enables a proof or verification of authenticity of the intraoral scanning device certificate, such as verification of the signer legitimacy. The digital signature is optionally generated by the manufacturing device using a client device customization private key. The intraoral scanning device may be configured to verify the digital signature of the client device certificate when receiving the (encrypted or unencrypted) client device certificate comprising the digital signature (i.e., receiving the authentication message comprising the encrypted client device certificate, and obtaining a decrypted version of the client device certificate). The digital signature is verifiable by the intraoral scanning device using a corresponding client device customization public key. If the digital signature is not successfully verified using the alleged public key, the intraoral scanning device may disregard the client device certificate and/or abort normal operation. This may provide the advantage that the intraoral scanning device rejects a client device certificate that is tampered or received from unauthenticated parties. The communication with the intraoral scanning device may thus be robust against impersonation, modification, and masquerading attacks.

The authentication message may comprise an authentication type identifier. To select an intraoral scanning device key from a plurality of intraoral scanning device keys may be based on the authentication type identifier. An authentication type identifier may be indicative of a client device type identifier and/or a certificate type identifier, e.g., of the (encrypted) client device certificate. The client device may be attributed a client device type corresponding to a model, category or type of client devices, such as a customization type, e.g. a computer product model, category or type configured for customizing the intraoral scanning device, a USB dongle product model, category or type configured for customizing the intraoral scanning device. A client device type identifier may refer to an identifier indicative of a client device type. A client device type identifier may uniquely identify a client device type. A client device type identifier may identify a type which the client device belongs to. The client device type identifier may be comprised in the client device certificate. The intraoral scanning device may be configured to select the intraoral scanning device key corresponding to the authentication type identifier and/or the authentication key identifier.

Customizing the intraoral scanning device implies that a customization part of the memory can be in read and/or writ mode. Customizing the intraoral scanning device implies that a firmware part of the memory is write-protected. The customization part of the memory may comprise setting data, such as power management settings, configuration of a user interface of the intraoral scanning device and/or settings of an optical unit of the intraoral scanning device.

The optical unit may include one or more light projectors, one or more optical components, and one or more image sensors.

The user interface of the intraoral scanning device may include at least a touch sensor, at least a touch button, at least a light emitting diode, a haptic sensor, and/or an accelerometer.

The client device data may include customization data which include setting data, such as power management settings, configuration of a user interface of the intraoral scanning device and/or settings of an optical unit of the intraoral scanning device. The client device data may include improved feature updates, new feature updates relating to an operating software system, a FPGA or other electronic/digital hardware of the intraoral scanning device.

The client device data may comprise an encrypted client device certificate; and the processing unit may be configured to generate a certificate key based on the selected intraoral scanning device key and/or the session identifier. To verify the client device data may comprise to decrypt the encrypted client device certificate with the certificate key to obtain a decrypted version of the encrypted client device certificate. The encrypted client device certificate may be generated by the client device using an encryption algorithm and a certificate key.

The intraoral scanning device may be configured to decrypt the encrypted client device certificate using a certificate key, a common secret and/or an intraoral scanning device key. The certificate key may be based on a common secret and/or a certificate value. The intraoral scanning device may be configured to obtain and/or generate the common secret based on an intraoral scanning device key, such as the selected intraoral scanning device key. For example, to generate the common secret based on the intraoral scanning device key, the intraoral scanning device may retrieve from the memory unit the intraoral scanning device key and/or the intraoral scanning device certificate from the memory unit, the intraoral scanning device certificate comprising an intraoral scanning device key, which is to be used for deriving the common secret. The intraoral scanning device may be configured to store the common secret in the memory unit, so as to e.g., retrieve the common secret from the memory unit when needed.

The intraoral scanning device being configured to receive client device data, or a linking request may be scheduled for a specific time on a day when the intraoral scanning device will not be used. The scheduling may be determined by the processing unit based on historical usage time of the intraoral scanning device and a machine learning model. The machine learning model receives timestamps from a clock in the intraoral scanning device and input information about when the intraoral scanning device is being used in a scanning session. The machine learning model includes a training data set which includes historical usage time of the intraoral scanning device being in the scanning session. Based on the machine learning model and a timestamp defining the time of the day the processing unit will know when to be configured to receive the client device data. The advantage of the scheduling is that a valid authenticated mode request will not interfere the work of the dentist with the intraoral scanning device. Furthermore, when being placed into the customization mode, the intraoral scanning device can be programmed to do time consuming updates within specific time-period(s). For example, an update which last more than 30 mins will automatically be planned to be performed in a time-period of more than 30 mins where the intraoral scanning device will not be used, such as outside the working hours or during a break of the dentist/clinic.

The processing unit may be configured to place the intraoral scanning device into the requested mode if authentication of the mode request succeeds and if a timestamp is within a time-period. The timestamp is generated by a clock of the intraoral scanning device and received by the processing unit.

The processing unit may include a machine learning model that includes a training data set which includes historical data that relates to usage time of the intraoral scanning device being in a scanning session, and wherein the machine learning model receives a timestamp from a clock in the intraoral scanning device and input information about when the intraoral scanning device is being used in a scanning session, and the processing unit may then be configured to receive a linking request or client device data based on an output of the machine learning model. The output of the machine learning model is a trigger for the processing unit to know when to be in a state for receiving a linking request and client device data

The intraoral scanning device may be configured to generate the common secret based on a session identifier using the processing unit and to store the common secret in the memory unit. For example, the intraoral scanning device may generate a common secret based on an intraoral scanning device key, e.g., the selected intraoral scanning device key, and a session identifier. The intraoral scanning device may generate the common secret CS, e.g., as follows:

$$CS = hash ( IOS\_KEY , S\_ID ) ,$$

where hash is a hash function, IOS_KEY is the (selected) intraoral scanning device key and S_ID is a session identifier. The session identifier may be generated by the intraoral scanning device upon reception of a linking request. The session identifier may comprise a random or pseudo random number of a defined length. The common secret may be used as a certificate key in one or more exemplary intraoral scanning devices.

The certificate key may be based on the common secret, e.g., generated by performing a hash function on the common secret and/or a certificate value. The intraoral scanning device may then generate the certificate key e.g., as follows:

$$C\_KEY = hash ( CS , C\_VAL ) ,$$

where hash is a hash function, CS is the common secret and C_VAL is a certificate value. The certificate value may be a predefined value or string, such as “certificate”.

In one or more exemplary intraoral scanning devices, the certificate key may optionally be generated by performing a hash function on the intraoral scanning device key and the session identifier. The intraoral scanning device may decrypt the encrypted client device certificate (part of the client device data) using the certificate key generated by the intraoral scanning device and obtain the decrypted version of the client device certificate. The intraoral scanning device may verify the content of the decrypted version of the client device certificate.

In one or more exemplary intraoral scanning devices, to verify the client device data comprises to determine if the authentication key identifier matches a client device key identifier of the client device certificate, and verification fails if no match is determined.

The intraoral scanning device may be configured to verify that the authentication key identifier matches a corresponding client device key identifier comprised in the client device certificate. The intraoral scanning device may be configured to verify that the authentication key identifier has a value that is equal to the client device key identifier comprised in the client device certificate. For example, the intraoral scanning device may be configured to verify that the authentication key identifier matches a corresponding client device key identifier comprised in the decrypted version of the client device certificate. In one or more exemplary intraoral scanning devices, to verify the client device data comprises to determine if a client device type identifier of the client device certificate is valid and verification fails if the client device type identifier of the client device certificate is not valid. For example, the intraoral scanning device may be configured to verify that the authentication type identifier matches a corresponding client device type identifier comprised in the decrypted version of the client device certificate.

In one or more exemplary intraoral scanning devices, to determine if a client device type identifier of the client device certificate is valid comprises to determine if the client device type identifier is black-listed, wherein the client device type is not valid if the client device type identifier is black-listed, e.g., appears on a list of black-listed client device types. In one or more exemplary intraoral scanning devices, to determine if a client device type identifier of the client device certificate is valid comprises to determine if the client device type identifier is allowed, wherein the client device type is valid if the client device type identifier is allowed, e.g., appears on a list of allowed client device types. For example, the client device type identifier of the client device may be valid if the authentication type identifier matches a corresponding client device type identifier comprised in the decrypted version of the client device certificate.

In one or more exemplary intraoral scanning devices, to verify the client device data comprises to verify a digital signature of the client device certificate, and verification fails if the digital signature is not verified. For example, the client device data comprises a digital signature appended to it to protect integrity of the client device data. Verifying a digital signature comprises e.g., computing a comparison result based on the digital signature and a corresponding client device public key and comparing the comparison result to the received client device data/client device certificate. The corresponding client device public key may be retrieved by the intraoral scanning device from the memory unit, a remote data storage unit, and/or the server device. The digital signature may be verified as valid, or the verification is successful when the digital signature raised to the power of the client device public key is identical to the received client device data.

In one or more exemplary intraoral scanning devices, the client device certificate comprises a signing device identifier and/or a client device identifier. The client device identifier refers to an identifier identifying a client device. The client device identifier may for example comprise a medium access control, MAC, address of the client device, and/or a serial number of the client device. The intraoral scanning device may be configured to verify the client device data by determining if the signing device identifier and/or the client device identifier are valid. For example, the intraoral scanning device may be configured to determine if the signing device identifier is valid by verifying that the signing device identifier is not black-listed. For example, the intraoral scanning device may be configured to determine if the client device identifier is valid by verifying that the client device identifier is not black-listed. The client device identifier allows for example the intraoral scanning device to identify the client device amongst a plurality of client devices. Verification fails if the signing device identifier and/or the client device identifier are not valid. For example, if the intraoral scanning device determines that the signing device identifier and/or the client device identifier are black-listed, the signing device identifier and/or the client device identifier are not valid and verification fails.

In one or more exemplary intraoral scanning devices, the processing unit may be configured to receive an additional authentication message. The additional authentication message may comprise client device data and/or an authentication device identifier. The authentication device identifier may refer to an identifier enabling authentication of the client device, such as a client device identifier comprised in an authentication message. For example, the authentication device identifier comprises a serial number, a medium access control, MAC, address, or any combination thereof. The intraoral scanning device may be configured to verify the authentication message and authenticate the client device sending the authentication message. The processing unit may be configured to obtain a common secret based on the authentication device identifier from the memory unit. The memory unit may have client device identifiers associated with common secrets stored thereon. The processing unit may then be configured to retrieve the corresponding common secret based on the authentication device identifier. The common secret has been generated and stored earlier at e.g., an initial round of authentication of a returning client device. Thus, once the client device authenticated, the processing unit can just retrieve the corresponding common secret. This provides a faster subsequent authentication and avoids having to regenerate the common secret for computing the additional certificate key, and thus saves the corresponding power consumption. The processing unit may be configured to generate an additional certificate key from the common secret; and to verify the client device data based on the additional certificate key. For example, the processing unit may generate the additional certificate key by computing a hash value based on the common secret and a certificate value. As described above, the processing unit may be configured to verify the client device data based on the additional certificate key by verifying the integrity of the client device data, such as verifying a MAC and/or a digital signature of the client device data. The processing unit is configured to verify the client device data based on the additional certificate key by decrypting the client device data using the additional certificate key (as a decryption key) when the client device data is received encrypted. The processing unit is configured to verify the client device data by verifying the content of the client device data. The processing unit may be configured to verify the client device data based on the additional certificate key by comparing the client device data with data stored in the memory unit.

In one or more exemplary intraoral scanning devices, the processing unit may be configured to generate an offline session key based on the common secret and the session identifier, and the processing unit may be configured to communicate with the client device using the offline session key. An offline session key may be used to secure offline communication between the intraoral scanning device and a client device. Offline communication refers to a communication that does not involve any other network device (e.g., a server device). To generate an offline session key may comprise to generate an offline key based on the common secret (e.g., perform a hash function of the common secret and an offline value), and to compute the offline session key based on the offline key and the session identifier (e.g. perform a hash function of the offline key and the session identifier). The offline session key is used by the intraoral scanning device and the client device to secure (e.g., encrypt) the intraoral scanning device data communicated between the intraoral scanning device and the client device.

In one or more exemplary intraoral scanning devices, the authentication message comprises an authentication token identifier, and the processing unit may be configured to store the authentication token identifier in the memory unit and to link the authentication token identifier with the common secret. The authentication token identifier may be indicative of enabling a token-based authentication at the intraoral scanning device, i.e., when the intraoral scanning device receives an authentication token identifier from an authenticated client device, it may enable token-based authentication in future communication with the same client device by storing e.g. an indicator such as a flag in relation with the common secret and the client. For example, the intraoral scanning device receiving the authentication token identifier may be configured to indicate to the processing unit to enable token-based authentication by storing and/or linking the token identifier with the common secret generated for the same client device, such as by storing and/or linking the token identifier with the common secret and the client device identifier of the same client device in e.g., a table. Token identifiers and token-based authentication may be used for intraoral scanning device management, such as to group intraoral scanning devices within a dental clinic and permit further customization with minimal or no user physical interaction/intervention as well as possibly simpler and faster client device authentication. The client device for example accesses securely a data storage where the token identifier is securely stored in a first session, retrieves the credential and keying material to perform token-based authentication in a subsequent session. This way, any client device in e.g., a dental clinic can be used to perform updates of the intraoral scanning device in a secure way using token-based authentication.

In one or more exemplary intraoral scanning devices, the processing unit may be configured to receive a further authentication message comprising client device data, an authentication type identifier, an authentication key identifier and/or an authentication session token identifier. The further authentication message may comprise an authentication device identifier. The processing unit may be configured to find in the memory unit the common secret linked to the client device type identifier and/or the client device identifier of the client device that sends the further authentication message based on locating the stored client device type identifier corresponding to the authentication type identifier and/or locating the stored client device identifier corresponding to the authentication device identifier. The processing unit may be configured to obtain a common secret based on the authentication type identifier: to generate a token key based on the common secret; and to generate a session token identifier based on the token key and the session identifier. The processing unit may have generated in an earlier session with the client device a common secret to e.g., establish a certificate key and may have stored and linked the common secret to the client device type identifier and/or the client device identifier. The processing unit may then be configured to obtain the common secret based on the authentication type identifier and/or the authentication client identifier corresponding to the stored client device type identifier and/or client device identifier. The processing unit may be configured to generate a token key by performing a hash function on the common secret and a token value (such as a pre-defined arbitrary string or a pre-defined arbitrary value). The processing unit may be configured to generate a session token identifier based on the token key and the session identifier by generating a session identifier, and by performing a hash function on the token key and the session identifier. The processing unit may be configured to verify the authentication session token identifier based on the session token identifier. The processing unit may be configured to verify the authentication session token identifier by comparing the authentication session token identifier and the generated session token identifier. For example, if the processing unit determines that the authentication session token identifier matches the generated session token identifier, the verification is successful and the processing unit may proceed with no user physical intervention and continue to verify the client device data provided in the further authentication message. The client device data may comprise a client device certificate. The intraoral scanning device may verify the client device certificate (and/or check against a blacklist) for any customization or updates to be allowed. The verified authentication token identifier may for example be used to indicate to the intraoral scanning device that the client device holds the previous shared token key and therefore is allowed to customize exactly this intraoral scanning device without physical intraoral scanning device user intervention.

In one or more exemplary intraoral scanning devices, the processing unit may be configured to generate a session key based on the session identifier and the intraoral scanning device key, and the processing unit may be configured to receive and authenticate session data based on the session key. To generate a session key based on the session identifier and the intraoral scanning device key may comprise computing the session key by generating a common secret based on the intraoral scanning device key and the session identifier and optionally generating a hash value of the common secret and a session value, the generated hash value corresponding to the session key. For example, the processing unit may be configured to authenticate session data based on the session key by verifying a MAC generated with the session key and/or by decrypting session data using the session key. The present disclosure relates to a method of operating an intraoral scanning device comprising a processing unit configured to process intraoral scan data of a patient and provide 2D image data and/or 3D image data, a memory unit, and a wireless interface, such as a method for controlling communication of an intraoral scanning device, such as a method for enabling secure intraoral scanning device communication. The method comprises receiving a linking request for a session via the wireless interface. The linking request may comprise an authentication key identifier and/or an authentication type identifier, in order to permit the intraoral scanning device to perform authentication at this early stage the linking request and the client device sending the linking request. This may provide a level of access control. The method comprises obtaining a session identifier, e.g., with the intraoral scanning device. Obtaining a session identifier may comprise generating a session identifier, such as by generating a random or pseudo-random number. For example, the processing unit generates a random or pseudo-random number of a predetermined length, e.g., 16 bytes, 32 bytes, 64 bytes etc., to be used as a session identifier. Obtaining a session identifier may comprise retrieving a session identifier from the memory unit. The method may comprise storing the session identifier in the memory unit. For example, storing the session identifier in the memory unit comprises storing the session identifier at a memory address of the memory unit, and/or in memory cells of the memory unit, such as in designated memory cells and/or at designated addresses.

The method comprises transmitting via the wireless interface a linking response comprising an intraoral scanning device identifier and the session identifier. Transmitting the linking response may comprise generating the linking response by including the session identifier and the intraoral scanning device identifier and transmitting the thus generated linking response to e.g., the client device.

The method comprises receiving, via the wireless interface, an authentication message. The authentication message comprises an authentication key identifier and client device data. The method may comprise receiving, via the wireless interface, an authentication message from a client device. For example, the intraoral scanning device receives the authentication message from the client device in order to establish a communication session. The client device data may comprise a client device certificate, customization data, intraoral scanning device operating parameters, and/or firmware data. The authentication key identifier may be an identifier that may be used to verify if the client device provides an authentication key identifier acceptable by the intraoral scanning device.

The method comprises selecting an intraoral scanning device key from a plurality of intraoral scanning device keys in the memory unit, based on the authentication key identifier. When the authentication key identifier matches the intraoral scanning device key identifier held by the intraoral scanning device and/or is indicative of an intraoral scanning device key of the intraoral scanning device, the processing unit may be configured to use the authentication key identifier as a key identifier indicating which intraoral scanning device key is to be used as keying material in the session. Selecting an intraoral scanning device key from a plurality of intraoral scanning device keys in the memory unit may be based on the authentication key identifier and an authentication type identifier. The authentication type identifier may be received in plaintext by the intraoral scanning device, and/or as client device type identifier in the client certificate (encrypted or decrypted). For example, the processing unit selects an intraoral scanning device key which the authentication key identifier and the authentication type identifier indicate.

The method comprises verifying the client device data, based on the selected intraoral scanning device key. Verifying the client device data may be based on an intraoral scanning device certificate or at least parts thereof. Further, the method comprises terminating the session if verification fails. Verifying the client device data based on the selected intraoral scanning device key may comprise verifying the integrity of the client device data based on the selected intraoral scanning device key, such as verifying a MAC and/or a digital signature comprised in the client device data. Verifying the client device data based on the selected intraoral scanning device key may comprise decrypting the client device data using the selected intraoral scanning device key (as keying material to derive a decryption key or as a decryption key) when the client device data is received encrypted. Verifying the client device data based on the selected intraoral scanning device key may comprise verifying the client device data by comparing the received client device data, e.g., decrypted client device certificate, with data stored in the memory unit. For example, verification fails if integrity of the client device data is detected as corrupted by e.g., verifying a MAC or a digital signature, if decryption fails, and/or if comparison of the received client device data with data stored in the memory unit shows a mismatch.

The authentication message optionally comprises an authentication type identifier. An authentication type identifier may be indicative of a client device type identifier and/or a certificate type identifier. Selecting an intraoral scanning device key from a plurality of intraoral scanning device keys may be based on the authentication type identifier. Selecting the intraoral scanning device key may be based on the authentication type identifier provided in the authentication message and/or the authentication key identifier verified.

The client device data may comprise a client device certificate (such as an encrypted client device certificate), an authentication key identifier, and/or an authentication type identifier. The client device may be assigned a client device certificate.

The method may comprise generating a certificate key based on the selected intraoral scanning device key and/or the session identifier; and verifying the client device data may comprise decrypting the encrypted client device certificate with the certificate key to obtain a decrypted version of the encrypted client device certificate. Decrypting the encrypted client device certificate with the certificate key may comprise decrypting the encrypted client device certificate using a certificate key, a common secret and/or an intraoral scanning device key, such as generating a certificate key based on a common secret and processing the encrypted client certificate using a decryption function and a certificate key. The certificate key may be based on a common secret and/or a certificate value. Generating a certificate key may comprise obtaining or generating the common secret based on the selected intraoral scanning device key. For example, generating the common secret based on the intraoral scanning device key comprises retrieving the intraoral scanning device certificate from the memory unit, the intraoral scanning device certificate comprising the selected intraoral scanning device key, and/or retrieving the selected intraoral scanning device key from the memory unit. The method may comprise generating the common secret based on a session identifier and/or the intraoral scanning device key. For example, the common secret CS is generated based on a selected intraoral scanning device key and a session identifier, e.g., as follows:

$$CS = hash ( IOS\_KEY , S\_ID ) ,$$

where hash is a hash function, IOS_KEY is the selected intraoral scanning device key and S_ID is a session identifier. The session identifier may comprise a random or pseudo random number of a defined length. The common secret may be used as a certificate key in one or more exemplary intraoral scanning devices. The intraoral scanning device may be configured to store the common secret in the memory unit, so as to e.g., retrieve the common secret from the memory unit when needed.

Generating a certificate key may comprise performing a hash function on the common secret and/or a certificate value. The intraoral scanning device may then generate the certificate key e.g., as follows:

$$C\_KEY = hash ( CS , C\_VAL ) ,$$

where hash is a hash function, CS is the common secret and C_VAL is a certificate value. The certificate value may be a predefined value or string, such as “certificate”.

In one or more exemplary methods, generating a certificate key comprises performing a hash function on the intraoral scanning device key and the session identifier. Stated differently, the common secret may be used as a certificate key if the client device has also used the common secret as certificate key to encrypt the client device certificate.

Verifying the client device data may comprise decrypting the encrypted client device certificate using the certificate key generated by the intraoral scanning device and obtaining the decrypted version of the client device certificate.

In one or more exemplary methods, verifying the client device data may comprise verifying a content of the decrypted version of the client device certificate. For example, verifying the client device data comprises determining if the authentication key identifier matches a client device key identifier of the client device certificate, and verification fails if no match is determined.

In one or more exemplary methods, verifying the client device data comprises determining if a client device type identifier of the client device certificate is valid and verification fails if the client device type identifier of the client device is not valid. For example, an authentication type identifier is sent in plain text in the authentication message, the authentication type identifier sent in plain text is valid if the authentication type identifier matches a corresponding client device type identifier comprised in the decrypted version of the client device certificate. For example, determining if a client device type identifier of the client device certificate is valid may comprise determining if the client device type identifier of the client device certificate is comprised in a list of authorized client devices.

In one or more exemplary methods, determining if a client device type identifier of the client device certificate is valid comprises determining if the client device type identifier is black-listed, wherein the client device type is not valid if the client device type identifier is black-listed, e.g., appears on a list of black-listed client device types. In one or more exemplary methods, determining if a client device type identifier of the client device certificate is valid comprises determining if the client device type identifier is allowed, wherein the client device type is valid if the client device type identifier is allowed, e.g., appears on a list of allowed or authorized client device types.

In one or more exemplary methods, verifying the client device data comprises verifying a digital signature of the client device certificate, and verification fails if the digital signature is not verified. For example, the client device data comprises a digital signature appended to it to protect integrity of the client device data. Verifying a digital signature comprises e.g., computing a comparison result based on the digital signature and a corresponding public key and comparing the comparison result to the received client device data. The digital signature may be verified as valid, or the verification may be successful when the digital signature raised to the power of the public key is identical to the received client device data.

In one or more exemplary methods, the client device certificate comprises a signing device identifier and/or a client device identifier and verifying the client device data comprises determining if the signing device identifier and/or the client device identifier is valid and wherein verification fails if the client device identifier of the client device and/or the signing device identifier is not valid.

In one or more exemplary methods, determining if a client device identifier of the client device certificate is valid comprises determining if the client device identifier is black-listed, wherein the client device identifier is not valid if the client device identifier is black-listed, e.g., appears on a list of black-listed client devices. In one or more exemplary methods, determining if a client device identifier of the client device certificate is valid comprises determining if the client device identifier is allowed, wherein the client device type is valid if the client device identifier is allowed, e.g., appears on a list of allowed or authorized client devices.

In one or more exemplary methods, the method comprises receiving an additional authentication message comprising client device data and/or an authentication device identifier. The method may further comprise obtaining, from the memory unit, a common secret based on the authentication device identifier, generating an additional certificate key from the common secret, and verifying the client device data based on the additional certificate key.

In one or more exemplary methods, the method comprises generating an offline session key based on the common secret and the session identifier and communicating with the client device using the offline session key.

In one or more exemplary methods, the method comprises receiving a further authentication message comprising client device data, an authentication type identifier, an authentication key identifier and/or an authentication session token identifier. The further authentication message may comprise an authentication device identifier. The method may comprise finding or determining in the memory unit the common secret linked to the client device type identifier and/or the client device identifier of the client device that sends the further authentication message based on locating the stored client device type identifier corresponding to the authentication type identifier and/or locating the stored client device identifier corresponding to the authentication device identifier. The method may comprise obtaining a common secret based on the authentication type identifier: generating a token key based on the common secret; and generating a session token identifier based on the token key and the session identifier. The processing unit may have generated in an earlier session with the client device a common secret to e.g., establish a certificate key and may have stored and linked the common secret to the client device type identifier and/or the client device identifier. The method may comprise obtaining the common secret based on the authentication type identifier and/or the authentication client identifier corresponding to the stored client device type identifier and/or client device identifier. The method may comprise generating a token key by performing a hash function on the common secret and a token value (such as a pre-defined arbitrary string or a pre-defined arbitrary value). The method may comprise generating a session token identifier based on the token key and the session identifier by generating a session identifier, and by performing a hash function on the token key and the session identifier. The method may comprise verifying the authentication session token identifier based on the session token identifier. The method may comprise verifying the authentication session token identifier by comparing the authentication session token identifier and the generated session token identifier. For example, if it is determined that the authentication session token identifier matches the generated session token identifier, the verification is successful and the processing unit may proceed with no user physical intervention and continue to verify the client device data provided in the further authentication message. The client device data may comprise a client device certificate. The intraoral scanning device may verify the client device certificate (and check against a blacklist) for any customization to be allowed. The verified authentication token identifier may for example be used to indicate to the intraoral scanning device that the client device holds the previous shared token key and therefore is allowed to customize exactly this intraoral scanning device without physical intraoral scanning device user intervention.

In one or more exemplary methods, the method comprises generating a session key based on the session identifier and the intraoral scanning device key, receiving, and authenticating session data based on the session key.

### Dental System, Intraoral Scanning Devices and Method of Securing Communication for a User Application:

Furthermore, the present disclosure relates to a dental system comprising a server device and an intraoral scanning device system, wherein the intraoral scanning device system comprises an intraoral scanning device and an external device. In particular, the present disclosure relates to devices for securing communication for a user application on accessory external device of a dental system comprising an intraoral scanning device, and a method of securing communication for a user application on accessory external device of a dental system comprising an intraoral scanning device.

An even further aspect of the present disclosure is to improve security in dental system communication. The dental system comprises a server device, an external device having a user application installed thereon and an intraoral scanning device. The server device may be controlled by the intraoral scanning device manufacturer. The server device may be a distributed server device, i.e., a server device with distributed processor. Namely, the method, user application and server device disclosed herein enables dental system communication that is robust against security threats, vulnerabilities, and attacks by implementing appropriate safeguards and countermeasures, such as security mechanisms, to protect against threats and attacks. The present disclosure relates to dental system communication that is robust against replay attacks, unauthorized access, battery exhaustion attacks, and man-in-the-middle attacks.

Yet another aspect of the present disclosure is to improve security of an intraoral scanning device. Security comprises in assessing threats, vulnerabilities and attacks and developing appropriate safeguards and countermeasures to protect against threats and attacks. The present disclosure relates to an intraoral scanning device comprising a processing unit configured to process intraoral scan data of a patient and provide 2D image data and/or 3D image data.

It is an important advantage of the present disclosure that the risk of user sensitive data, such as intraoral scanning device settings and/or user specific software updates, being sent to or shared with third party user applications or otherwise corrupted user applications is heavily reduced or eliminated.

Further, the present disclosure allows an intraoral scanning device manufacturer to securely keep and maintain updated and correct information on user applications. Even further, a server device or an intraoral scanning device manufacturer can keep updated information on and link user applications with specific intraoral scanning devices.

According to the aspects, a method of securing communication for a user application installed on an external device of a dental system comprising an intraoral scanning device, a server device, and the external device, is disclosed. The securing communication for the user application comprises obtaining challenge data in the server device: transmitting the challenge data from the server device to the user application installed on the external device: transmitting a challenge request comprising the challenge data from the user application to the intraoral scanning device: receiving a challenge response comprising response data from the intraoral scanning device: forwarding the response data from the user application to the server device: verifying the response data in the server device based on the challenge data; and approving the user application in the server device if verifying the response data is successful.

According to the aspect, a dental system comprising a server device and an intraoral scanning device system, is disclosed. The intraoral scanning device system comprising an external device and an intraoral scanning device, the server device being configured for securing communication for a user application installed on the external device. The server device may be configured to approve the user application, wherein to approve the user application comprises to obtain challenge data: transmit the challenge data to the user application: receive a response message comprising response data from the user application, the response data comprising an intraoral scanning device identifier: verify the response data based on the challenge data; and approve the user application if the response data are verified. The external device may comprise a processing unit, a memory unit; and a wireless interface, wherein the user application is configured to secure communication for the user application. The secure communication for the user application may be comprised to obtain challenge data from the server device: transmit a challenge request comprising the challenge data to the intraoral scanning device of the intraoral scanning device system: receive a challenge response comprising response data from the intraoral scanning device; and forward the response data to the server device.

As used herein the term “identifier” refers to a piece of data that is used for identifying, such as for categorizing, and/or uniquely identifying. The identifier may be in a form of a word, a number, a letter, a symbol, a list, an array, or any combination thereof. For example, the identifier as a number may be in the form of an integer, such as unsigned integer, uint, with a length of e.g., 8 bits, 16 bits, 32 bits, or more, such as an array of unsigned integers. An identifier may have a length of several bytes. For example, an intraoral scanning device identifier may have a length of 20 bytes.

The external device comprises a memory unit and a wireless interface respectively connected to a processing unit. The memory unit may include removable and non-removable data storage units including, but not limited to, Read Only Memory (ROM), Random Access Memory (RAM), etc. The memory unit has a user application stored thereon. The wireless interface comprises an antenna and a wireless transceiver, e.g., configured for wireless communication at frequencies in the range from 2.4 to 2.5 GHZ, 2.4 GHz to 5 GHZ, about 2.45 GHz or about 5 GHz. The wireless interface may be configured for communication, such as wireless communication, with the intraoral scanning device comprising an antenna and a wireless transceiver.

The user application may be a dental software configured for handling an intraoral scanning device. The user application may be a dental software configured to receive 2D image data and/or 3D image data, and visualize the image data on a graphical user in real-time.

The method comprises obtaining challenge data in a server device. Obtaining challenge data may comprise generating the challenge data, e.g., based on a default challenge value and/or a timestamp. Accordingly, the server device may be configured to generate the challenge data, e.g., based on a default challenge value and/or a timestamp. The server device may be configured to generate the challenge data at a certain interval, such as every 5 minutes, every 10 minutes, or every 30 minutes. While a short time between generation of (different) challenge data may increase security, a too short time between generation of (different) challenge data may set too high timing requirements for the user application/intraoral scanning device, which in turn leads to unnecessary faulty verifications and requires power-consuming challenge-response generation in the intraoral scanning device. The challenge data may be random or pseudo-random. The challenge data may comprise at least 8 bytes, such as at least 16 bytes. The challenge data may be a 16-bytes value. The server device may be configured to generate the challenge data based on a look-up table and/or a function, e.g., having a timestamp as input. Obtaining challenge data based on a timestamp value enables and/or provides challenge data with a built-in validity period. Obtaining challenge data with a given interval enables and/or provides challenge data with a built-in validity period.

The present disclosure relates to secure communication between entities of a dental system. The dental system comprises a server device and an intraoral scanning device system, the intraoral scanning device system comprising an external device and an intraoral scanning device. The external device forms a communication device to the intraoral scanning device. The external device is typically paired or otherwise wirelessly coupled to the intraoral scanning device.

Obtaining challenge data may comprise storing the challenge data in the server device. The server device may be configured to delete the challenge data after verifying the response data. The method may comprise deleting the challenge data after a certain period of time and/or replacing the challenge data with new challenge data.

The method comprises transmitting the challenge data from the server device to the user application.

The method comprises transmitting a challenge request comprising the challenge data from the user application to the intraoral scanning device.

The method comprises receiving a challenge response, e.g., in the user application, the challenge response comprising response data from the intraoral scanning device. The response data may comprise at least 8 bytes, such as at least 16 bytes or at least 32 bytes. The response data may have a length in the range from 16 to 72 bytes. The response data may comprise an intraoral scanning device identifier. The response data may comprise a key identifier for enabling the server device to use or apply the correct keying material when verifying the response data. The response data may comprise intraoral scanning device challenge data generated in the intraoral scanning device.

The response data comprises a response value, e.g., a challenge response value, and/or intraoral scanning device data. The response data may comprise a checksum value based on the response value and/or the intraoral scanning device data. The response value may be based on the challenge data and/or intraoral scanning device data, e.g., an intraoral scanning device identifier. The response value may be generated based on one or more of the challenge data from the server device, an intraoral scanning device key identified by the key identifier, the intraoral scanning device identifier, and intraoral scanning device challenge data. The response value may be based on a static string. The response value may be encrypted using one or more of challenge data from the server device, a key identified by the key identifier, the intraoral scanning device identifier, and intraoral scanning device challenge data as keying material.

The method comprises forwarding the response data from the user application to the server device, e.g., in a response message. The response data, e.g., the response value of the response data, are verified in the server device based on the challenge data. Verifying the response data in the server device based on the challenge data may comprise calculating the challenge data, e.g., based on a default challenge value and/or a timestamp. Verifying the response data in the server device based on the challenge data may comprise retrieving the challenge data from a memory of the server device. Verifying the response data in the server device may be based on intraoral scanning device challenge data of the response data. Verifying the response data in the server device may be based on intraoral scanning device identifier of the response data. Verifying the response data may comprise calculating a verification value based on the challenge data from the server device and/or one or more of a key identified by the key identifier, intraoral scanning device challenge data, and intraoral scanning device identifier of the response data. Verifying the response data may comprise comparing the verification value with the response value. The response data may be verified (verifying is successful) if the verification value corresponds to the response value.

The method optionally comprises approving the user application in the server device if verifying the response data is successful. Thus, the server device regards the user application as a trusted entity in the system if verifying the response data is successful. In other words, the user application can be said to be whitelisted in the server device if verifying the response data is successful.

The method optionally comprises disapproving the user application in the server device if verifying the response data fails. Thus, the server device may regard the user application as an un-trusted entity in the system if verifying the response data is successful. The user application may be black-listed, e.g., for a certain period, in the server device if verifying the response data fails, e.g. if verifying the response data fails for a number of times, e.g. two, three or more. The method may comprise setting a user application status identifier to a value indicative of the user application not being approved if verifying the response data fails.

The method may comprise determining the response data, or at least a response value thereof, in the intraoral scanning device based on the challenge data and/or intraoral scanning device identifier of the intraoral scanning device. Thus, the intraoral scanning device may be configured to generate the response data based on the challenge data and/or an intraoral scanning device identifier. Response data, such as a response value, based on an intraoral scanning device identifier enables the server device to authenticate the intraoral scanning device. The response data optionally comprises or is indicative of an intraoral scanning device identifier. Thus, the server device can identify a specific intraoral scanning device.

In the method, receiving a challenge response comprising response data from the intraoral scanning device may be performed by the user application.

In the method, approving the user application comprises setting a user application status identifier to a value indicative of the user application being approved.

The method may comprise linking the user application to an intraoral scanning device, e.g., to the intraoral scanning device identifier of the intraoral scanning device, in a memory of the server device if verifying the response data is successful.

The method may comprise transmitting a request for challenge data from the user application. Thus, the user application and/or intraoral scanning device may be able to initiate the secure communication between the user application and the server device, e.g., if the user application is updated and/or if the external device and/or the user application is restarted, in turn increasing the security level.

The request for challenge data may be transmitted if a first approval criterion, e.g., in the user application, is fulfilled. The first approval criterion may comprise determining, e.g., in the user application, if the user application has been approved earlier, wherein the first approval criterion is fulfilled if the user application has not been approved earlier. The first approval criterion may be fulfilled if the user application is started for the first time, e.g., after installation of the user application and/or after repowering of the external device. The first approval criterion may be fulfilled if the user application has been updated to a new version.

The method may comprise storing an approval timestamp indicative of time of last approval: determining if a second approval criterion based on the approval timestamp is fulfilled; and initiate securing communication for the user application if the second approval criterion is fulfilled. Thereby is ensured that the server device approves/disapproves a user application with a certain frequency, further increasing the security in the dental system by keeping an updated user application database in the server device and to optimize dental system communication.

In the method, approving the user application may comprise transmitting intraoral scanning device settings specific for the intraoral scanning device to the user application. Approving the user application may comprise transmitting intraoral scanning device operating parameters specific for the intraoral scanning device to the user application.

The method may comprise not approving or disapproving the user application if response data are not received within an approval period, e.g., from obtaining challenge data or transmitting the challenge data. In one or more exemplary server devices/methods, the length of an approval period may be determined by a frequency of determining new challenge data. In one or more exemplary devices/methods, challenge data are calculated or generated with a given interval, such as every 5 minutes or every 10 minutes.

The method may comprise establishing a secure session between the user application and the intraoral scanning device and optionally transmitting the challenge request in the secure session, such as an integrity-protected, encrypted, authenticated, and/or mutually authenticated session. The challenge response may be received in the secure session.

The method may comprise establishing a secure session, such as an integrity-protected, encrypted, authenticated, and/or mutually authenticated session, between the server device and the user application, and optionally transmitting the challenge data in the secure session. The response data may be forwarded from the user application to the server device in the secure session.

The server device may be configured to determine if an approval criterion is fulfilled, the server device being configured to initiate securing communication for the user application if the approval criterion is fulfilled, wherein the approval criterion comprises a first approval criterion and a second approval criterion, and wherein the approval criterion is fulfilled if the first approval criterion and/or the second approval criterion is fulfilled. The second approval criterion may be fulfilled if the time since last approval is longer than an approval time threshold, e.g., one or more days, such as 7 days, 14 days. Thus, approval of a user application with a minimum frequency may be employed to ensure updated user application data in the server device.

The present disclosure also relates to a user application for an external device of a dental system. The external device may be a tablet computer, a dental clinic computer, or a computer. The user application is, when installed on the external device, configured to secure communication for the user application.

The user application may be configured to determine if a first approval criterion is fulfilled and to initiate securing communication for the user application if the first approval criterion is fulfilled, and wherein to obtain challenge data comprises to transmit a request for challenge data to the server device. The request for challenge data is a message requesting the server device to transmit challenge data to the user application. Thus, the user application and/or intraoral scanning device (via the user application) can actively initiate approval of the user application in the server device.

By enabling dental system entities to initiate securing communication for the user application, the approval procedures can be optimized, e.g., by enabling the approval procedure to be initiated only when necessary or when justified due to changes in the different entities in the dental system.

Client device for secure intraoral scanning device:

The functionality of an intraoral scanning device becomes increasingly advanced. Intraoral scanning device communication comprises wireless communication between an intraoral scanning device and external devices, such as a computers and tablets, which increases in complexity. Intraoral scanning device communication is exposed to many challenges in terms of security. A device communicating with an intraoral scanning device may be a legitimate device but may also be a rogue device. If communication with an intraoral scanning device does not permit to distinguish legitimate devices from rogue devices, this opens the door to a plethora of attacks, such as unauthorized access to the intraoral scanning device memory to write or change data. Any such attacks may result in a malfunction of the intraoral scanning device, e.g., a battery exhaustion attack.

However, an intraoral scanning device is a small device with strict constraints in terms of computational power, memory space, etc. Therefore, a device communicating with an intraoral scanning device cannot use an off-the-shelf security algorithm and protocol, at the risk of e.g., depleting the intraoral scanning device battery or degrading functions of the intraoral scanning device rendering the intraoral scanning quasi-useless.

Present intraoral scanning devices are part of a service infrastructure which includes communication between intraoral scanning devices, scan software for a specific service, and the provider of the service. The service could for example include manufacture of an aligner, a retainer, a crown, an implant, a bracer, a nightguard etc. For improving the usability of such an infrastructure for the dentist, minimal interaction between the infrastructure and the dentist is needed. One way of achieving this is by applying wireless communication between the intraoral scanning device and an external computer that is connected to a server that can forward the intraoral scan data to a service provider. Scan data of a patient can be characterized as being personal information, and therefore, there is a need for minimizing any risk of a third party stealing or corrupting the at least scan data. The scan data is characterized as personal information, and in some situations, other type of personal information is associated with the scan data, such as age, gender, location address, personal security number etc. In this example, a demand for improving the security of the wireless communication in the service infrastructure is needed.

An aspect of the present disclosure to provide a client device, and a method which seeks to mitigate, alleviate, or eliminate one or more of the above-identified deficiencies in the art and disadvantages singly or in any combination.

A further aspect of the present disclosure is to improve security in wireless communication with an intraoral scanning device that protects the intraoral scanning device against potential attacks, such as an improved client device, and a method of communication with an intraoral scanning device that improves security thereof.

According to the aspects, a client device for intraoral scanning device communication is disclosed. The client device may comprise a processing unit, a memory unit, and a wireless interface configured to receive 2D image data and/or 3D image data from an intraoral scanning device. The processing unit may be configured to send a session request for a session to the intraoral scanning device via the wireless interface, receive a session response from the intraoral scanning device via the wireless interface, the session response comprising an intraoral scanning device identifier. Furthermore, the processing unit may be configured to obtain a session key based on the session response. Additionally, the processing unit may be configured to determine intraoral scanning device data, generate session data based on the session key and the intraoral scanning device data, and send the session data to the intraoral scanning device via the wireless interface.

According to the aspects, a client device for intraoral scanning device communication is disclosed. The client device may comprise a processing unit, a memory unit, and a wireless interface configured to receive 2D image data and/or 3D image data from an intraoral scanning device. The processing unit may be configured to send a session request for a session to the intraoral scanning device via the wireless interface, receive a session response from the intraoral scanning device via the wireless interface, the session response comprising an intraoral scanning device identifier. Furthermore, the processing unit may be configured to obtain a session key based on the session response, wherein to obtain a session key comprises to establish a connection to a session key generator via the wireless interface, to send a session key request to the session key generator via the wireless interface, the session key request comprising the intraoral scanning device identifier, to receive a session key response from the session key apparatus via the wireless interface, and to determine the session key based on the session key response. Additionally, the processing unit may be configured to determine intraoral scanning device data, generate session data based on the session key and the intraoral scanning device data, and send the session data to the intraoral scanning device via the wireless interface.

According to the aspect, a method, performed in a client device, for intraoral scanning device communication, the client device comprising a processing unit, a memory unit and a wireless interface configured to receive 2D image data and/or 3D image date from an intraoral scanning device. The method may comprise sending a session request for a session to the intraoral scanning device via the wireless interface, receiving a session response via the wireless interface, obtaining a session key based on the session response, wherein obtaining a session key comprises establishing a connection to a session key apparatus, sending a session key request to the session key apparatus, receiving a session key response from the session key apparatus, and determining the session key based on the session key response. Furthermore, the method may comprise determining intraoral scanning device data, generating session data based on the session key and the intraoral scanning device data, and sending the session data to the intraoral scanning device via the wireless interface.

The intraoral scanning device is a handheld scanning device for scanning inside an oral cavity of a patient. The intraoral scanning device differs from other type of teeth scanning device in that the intraoral scanning device is a handheld scanning device which can easily be handled by one hand by a user, and which has now wired connection to any external device during scanning of an inside of an oral cavity of a patient. Therefore, the only attack which an intraoral scanning device may experience is via the wireless interface.

The method and the intraoral scanning device as disclosed provide secure configuration of the intraoral scanning device, such as secure access to the memory of the intraoral scanning device. It is an advantage of the present disclosure that the intraoral scanning device can only be configured or updated by authorized parties. The disclosed intraoral thus has the advantage of detecting and preventing any modification by unauthorized parties. The intraoral scanning device disclosed herein is advantageously protected against attacks such as spoofing attacks, man-in-the-middle attacks, and/or replay-attacks.

The intraoral scanning device is the key element in providing the needed level of security in wireless communication in a service infrastructure which at least includes the intraoral scanning device and a scan computer, or a dental software installed on a computer. It would not be possible for a third party to attack the wireless communication as this person needs to have the intraoral scanning device physically in its hand. It would not even be enough to have access to the scan computer or the dental software.

The method and the client device as disclosed provide a secure communication from the client device to the intraoral scanning device, such as provide to the client device a secure and/or authorized access to the memory of the intraoral scanning device. It is an advantage of the present disclosure that the communication between the client device and the intraoral scanning device is protected against any action or at least some actions from undesired parties. The disclosed client device thus has the advantage of allowing the intraoral scanning device to detect any modification by unauthorized parties. The client device provides a secure communication adapted to the intraoral scanning device, which in turn is able to communicate securely with legitimate parties such as the client device and to counterstrike attacks such as spoofing attacks, man-in-the-middle attacks, and/or replay-attacks.

The method as disclosed herein provides a secure configuration and/or update of an intraoral scanning device.

The present disclosure provides improved security of communication performed between the client device and an intraoral scanning device. Security comprises assessing threats, vulnerabilities and attacks and developing appropriate safeguards and countermeasures to protect against threats and attacks. The present disclosure provides an intraoral scanning device comprising a processing unit configured to process intraoral scan data of a patient and provide 2D image data and/or 3D image data. The 2D image data and/or 3D image data may include information about the anatomy of the oral cavity of the patient, such as teeth, gingival, bone level, and/or information about diagnostic indicators such as caries, bone loss, gingivitis, gingiva recession, periodontitis, bone loss, cracks, and occlusion.

The processing unit may be configured to obtain a session key which comprises to establish a connection to a session key apparatus via the wireless interface, to send a session key request to the session key apparatus via the wireless interface, the session key request comprising the intraoral scanning device identifier, to receive a session key response from the session key apparatus via the wireless interface, and to determine the session key based on the session key response.

The processing unit may be configured to obtain a session key which comprises validating the intraoral scanning device identifier based on a client device key and derive the session key based on the intraoral scanning device identifier.

The processing unit may be configured to process the received 2D image data and/or 3D image data for the purpose of visualizing the data on a display, for designing dental accessories, such as aligners, retainers, crowns, implants, bracers, nightguards etc., and/or for providing diagnostic data.

The 2D image data and/or the 3D image data may be image data configured to be visualizable on a display in a 2D or a 3D manner, respectively.

The term “client device” as used herein refers to a device that communicates with the intraoral scanning device. The client device may refer to a computing device acting as a client. The client device may comprise a customization device, a handheld device, a relay, a tablet, a personal computer, a mobile phone, and/or USB dongle plugged into a personal computer. The client device may control operation of the intraoral scanning device, either by sending customization data, intraoral scanning device operating parameters, and/or firmware data. The disclosed client device and method support the intraoral scanning device in combatting attacks such as unauthorized access or control of an intraoral scanning device, while still allowing access to legitimate parties such as the client device, for e.g., customization purposes, update purposes, maintenance purposes.

The intraoral scanning device may be operated in one or more modes. The one or more modes may include a first mode and/or a second mode. The one or more modes may include a third mode and/or a fourth mode. The one or more modes may include a default mode.

The client device may comprise a memory unit and a wireless interface respectively connected to the processing unit. The wireless interface may comprise a wireless transceiver, e.g., configured for wireless communication at frequencies in the range from 2.4 to 2.5 GHZ, 2.4 GHz to 5 GHZ, about 2.45 GHz or about 5 GHz. The wireless transceiver may be a Bluetooth transceiver, a Bluetooth Low Energy transceiver, or a Wireless Fidelity (WIFI) transceiver. The wireless interface may form a connection to one or more other devices such as a computer, and/or a scan computer, and/or a tablet and/or a smart phone.

In an embodiment, the wireless interface is configured for communication, such as wireless communication, with an intraoral scanning device comprising a wireless transceiver.

The processing unit may be configured to send a session request for a session to the intraoral scanning device via the wireless interface. The processing unit may be configured to receive a session response from the intraoral scanning device via the wireless interface, e.g., from an intraoral scanning device and/or a session key apparatus. The session response may comprise the intraoral scanning device identifier or an identifier derived therefrom. In an exemplary client device, the client device may receive the intraoral scanning device identifier during a pairing of the client device and the intraoral scanning device. Hence, the processing unit comprises e.g., a receive/send unit configured to send data such as the session request and/or receive data such as the session response via the wireless interface. The processing unit may be configured to obtain a session key based on e.g., the session response, such as to extract the session key from or based on the session response. Hence, the processing unit comprises an obtainer. The processing unit may retrieve the session key from a key depository, e.g., stored in the memory unit. The processing unit may be configured to obtain a session key, wherein to obtain a session key may comprise to establish a connection to a session key apparatus via the wireless interface. The processing unit may send a session key request to the session key apparatus such as a session key server via the wireless interface e.g., via a wireless communication link established between the client device and the session key apparatus via the wireless interface. The processing unit may receive a session key response from the session key apparatus via the wireless interface and may determine the session key based on the session key response.

The session response may comprise an intraoral scanning device identifier. The intraoral scanning device identifier may comprise a hardware number of the intraoral scanning device and/or a serial number of the intraoral scanning device. The client device may retrieve the session key from the session key apparatus by providing the intraoral scanning device identifier to the session key apparatus, e.g., as part of the session key request, and requesting the session key or an intraoral scanning device key from the session key apparatus and/or requesting the session key apparatus to decrypt the session response and/or the session key.

In one or more exemplary client devices, the processing unit configured to obtain the session key may be configured to establish a connection to a session key apparatus via the wireless interface, to send a session key request to the session key apparatus via the wireless interface, to receive a session key response from the session key apparatus via the wireless interface, and to determine the session key based on the session key response. The session key request may comprise the intraoral scanning device identifier. The connection to the session key apparatus may be a secure connection over a network, such as including a private and/or a public network.

The session key apparatus may be a customization accessory device: wherein the customization accessory device optionally comprises a storage device containing a list configured to provide a session key and/or a session key response based on a session key request.

The processing unit may be configured to determine intraoral scanning device data. Hence the processing comprises e.g., a determiner. The intraoral scanning device data comprises e.g., firmware, customization data, and/or intraoral scanning device operating parameters. Customization data may for example be setting data of the intraoral scanning device, such as power management settings, configuration settings, configuration of a user interface of the intraoral scanning device and/or settings of an optical unit of the intraoral scanning device. Firmware may refer to a computer program provided by the intraoral scanning device manufacturer, and to be installed on the intraoral scanning device to control the intraoral scanning device. Firmware is for example to be installed to upgrade the operations and capabilities of the intraoral scanning device.

The optical unit may include one or more light projectors, one or more optical components, and one or more image sensors.

The user interface of the intraoral scanning device may include at least a touch sensor, at least a touch button, at least a light emitting diode, a haptic sensor, and/or an accelerometer.

The session response may comprise an encrypted session key. The processing unit may be configured to determine the session key by retrieving the session key from the session key response.

In one or more exemplary client device, to determine the session key comprises retrieving an intraoral scanning device key from the session key response or from the memory unit and decrypting the encrypted session key based on the intraoral scanning device key. To determine the session key may comprise decrypting the encrypted session key with a global key. A global is e.g., a key common to a group of client devices. The processing unit may be configured to retrieve an intraoral scanning device key from the session key response and decrypt the encrypted session key based on the intraoral scanning device key. The processing unit may comprise a decrypt/encrypt unit. The intraoral scanning device key may be e.g., a symmetric key or a public key of a private-public key pair. The intraoral scanning device key may comprise an AES-128 bits key as a symmetric key. The use of a symmetric key as an intraoral scanning device key provides the advantage of being able to use hardware accelerators. The intraoral scanning device key may comprise a public key of a private-public key pair, such as a public key of a private-public key pair of an authorized discloser of the session key, such as of the client device or the session key apparatus.

The processing unit may be configured to determine the session key by including a decryption of the encrypted session key with a global key, i.e., to determine the session key may comprise decrypting the encrypted session key with a global key. The global key may be e.g., a symmetric key or a public key of a private-public key pair. The session key may be compliant with an encryption standard such as Advanced Encryption Standard, AES, RSA crypto-system, Triple Data Encryption Algorithm.

The processing unit may be configured to generate session data, e.g., including a message authentication code, based on the session key and the intraoral scanning device data. Hence the processing unit may comprise a generator. The processing unit may generate a message authentication code based on the session key and the intraoral scanning device data. The message authentication code may be included in the session data. The processing unit may be configured to generate session data based on an intraoral scanning device key. The processing unit may be configured to digitally sign the intraoral scanning device data, such as to digitally sign the intraoral scanning device data using a private key of the client device, and/or of a group of client devices. The processing unit may be configured to digitally sign the intraoral scanning device data using a private key obtained from the session key apparatus, e.g., as part of a session key response. The processing unit may generate a digital signature using a signature generation function and a private key of a client device and append the digital signature to the session data. The intraoral scanning device may then verify the digital signature when receiving the session data. If the digital signature is not successfully verified using the alleged public key of a client device, the intraoral scanning device may disregard the session data and/or terminate the session. This may provide the advantage that the client device supports the intraoral scanning device in rejecting session data tampered or received from unauthenticated parties and the communication with the intraoral scanning device may thus be robust against impersonation and masquerading attacks.

The processing unit may be configured to send the session data to the intraoral scanning device via the wireless interface, e.g., using the receive/send unit. The session data may comprise intraoral scanning device data encrypted with the session key. To encrypt session data with the session key, the client device may utilize any of the above encryption standards.

### Method of Controlling Access to Intraoral Scanning Device Services:

Furthermore, the present disclosure relates to a method of controlling an access of an intraoral scanning device services by clients.

An aspect of the present disclosure to provide a client device, and a method which seeks to mitigate, alleviate, or eliminate one or more of the above-identified deficiencies in the art and disadvantages singly or in any combination.

A further aspect of the present disclosure is to improve security in wireless communication with an intraoral scanning device that protects the intraoral scanning device against potential attacks, such as an improved client device, and a method of communication with an intraoral scanning device that improves security thereof.

An even further aspect of the present disclosure is to provide for a method of operating an intraoral scanning device, wherein access to intraoral scanning services by client devices is to be controlled in an efficient manner.

According to the aspects, a method of controlling access of a client device to a service of an intraoral scanning device is disclosed. The method may comprise the steps of requesting access of the client device to the service of the intraoral scanning device by providing a client device authenticator to the intraoral scanning device; authenticating the client device based on a validation of the provided client device authenticator by the intraoral scanning device. Furthermore, the method may comprise upon successful authentication, comparing a security level associated with the service requested by the client device with a highest security level assigned to the client device by the intraoral scanning device, wherein the security level is selected from a plurality of hierarchically structured security levels, and granting access of the client device to the service of the intraoral scanning device, if the requested security level is below or equal to the highest security level assigned to the client device.

The present disclosure is beneficial in that it allows to implement a service access control which is enforced on the intraoral scanning device at runtime without the need for an external entity and which provides for client specific service access, while having low resource requirements, taking into account the typically limited resources of intraoral scanning devices, in particular with regard to memory space, power consumption and computational effort.

Another client device may be configured to communicate with the intraoral scanning device via the client device through at least a wired communication link. The client device that communicates via the wireless interface to the intraoral scanning device may be a gateway to the intraoral scanning device. First, the another client device has to establish a connection to the client device through a key-exchange scheme. Another session request may then be send by the client device via the wireless interface to the intraoral scanning device. The processing unit of the client device may then receive a session response from the intraoral scanning device via the wireless interface, and a session key may then be obtained by the processing unit based on the session response. Session data to be communicated between the another client device and the intraoral scanning device may be generated based on the session key and the intraoral scanning device data. The session data is then communicated via the wireless interface between the client device and the intraoral scanning device and then via at least a wired connection between the another client device and the client device. In another example, the another session request may be part of the session request that is generated by the client device, and which means that only one session request is needed to be forward in order to establish a communication link between the another client device, the client device and the intraoral scanning device. The another client device may be located remotely from the clinic which operates the client device and the intraoral scanning device. In the another client device a dentist may be needed to monitor a scanning of a patient remotely, and this can be achieved with high security by configuring the client device to be a gateway between the another client device and the intraoral scanning device.

The another client device may be a second client device, and the client device may be a first client device, and a dental system may then include the first client device, at least the second client device and the intraoral scanning device.

The another client device, the client device and the intraoral scanning device may be part of a mesh network for sharing or distributing intraoral scanning data.

Client Device with Certificate and Related Method:

Furthermore, the present disclosure relates to a client device for intraoral scanning device communication and related method. In particular, a method of operating a client device for intraoral scanning device communication is disclosed.

An aspect of the present disclosure to provide a client device, and a method which seeks to mitigate, alleviate, or eliminate one or more of the above-identified deficiencies in the art and disadvantages singly or in any combination.

A further aspect of the present disclosure is to improve security in wireless communication with an intraoral scanning device that protects the intraoral scanning device against potential attacks, such as an improved client device, and a method of communication with an intraoral scanning device that improves security thereof.

There is a need for client device and method providing improved security for intraoral scanning device communication. Further, there is a need for devices and methods reducing the risk of an intraoral scanning and intraoral scanning function being compromised by a third party.

According to the aspects, a client device for intraoral scanning device communication is disclosed. The client device may comprise a processing unit, a memory unit, and a wireless interface. The memory unit may have a client device key, such as at least one client device key, and/or a client device certificate stored thereon. The processing unit may be configured to receive a connection response, e.g., comprising an intraoral scanning device identifier, via the wireless interface: generate one or more keys, e.g., including a certificate key, based on the intraoral scanning device identifier and/or the client device key: obtain an authentication message based on the certificate key and/or the client device certificate. To obtain the authentication message may optionally comprise to generate and/or obtain an encrypted client device certificate by encrypting the client device certificate, e.g., with the certificate key, and optionally to include the encrypted client device certificate in the authentication message. The processing unit may be configured to transmit the authentication message via the wireless interface.

The processing unit may be configured to obtain a session identifier. To generate one or more keys may comprise to generate an intraoral scanning device key based on the intraoral scanning device identifier and the client device key and may further comprise to generate a common secret based on the intraoral scanning device key and the session identifier.

The certificate key may be based on the common secret and a certificate value.

To generate one or more keys may comprise to generate a session key based on the intraoral scanning device identifier, the session identifier, and the client device key. The processing unit may be configured to transmit the session key to a customization device.

The session key may be based on the common secret and a session value.

The processing unit may further be configured to include an authentication key identifier indicative of the client device key in the authentication message.

The processing unit may further be configured to include an authentication type identifier in the authentication message.

The client device certificate may comprise one or more of:
a certificate type identifier;
a signing device identifier;
a client device type identifier;
a client device identifier;
a client device key identifier;
one or more hardware identifiers; and
a digital signature.

According to the aspects, a method of operating a client device for intraoral scanning device communication is disclosed, the client device comprising a memory unit having a client device key, such as at least one client device key, and/or a client device certificate stored thereon. The method comprises receiving a connection response, e.g., comprising an intraoral scanning device identifier via the wireless interface: generating one or more keys, e.g., including a certificate key, based on the intraoral scanning device identifier and/or the client device key; and obtaining an authentication message based on the certificate key and/or the client device certificate. Obtaining the authentication message optionally comprises generating an encrypted client device certificate, e.g., by encrypting the client device certificate with the certificate key, and optionally including the encrypted client device certificate in the authentication message. The method comprises transmitting the authentication message via the wireless interface.

The method may comprise obtaining a session identifier, and wherein generating one or more keys may comprise generating an intraoral scanning device key based on the intraoral scanning device identifier and the client device key, and generating a common secret based on the intraoral scanning device key and the session identifier.

The certificate key may be based on the common secret and a certificate value, or the method may comprise basing the certificate key on the common secret and a certificate value.

Generating one or more keys may comprise generating a session key based on the intraoral scanning device identifier, the session identifier, and the client device key. The method may further comprise transmitting the session key to a customization device.

The session key may be based on the common secret and a session value, or the method may comprise basing the session key on the common secret and a session value.

Obtaining the authentication message may comprise including an authentication key identifier indicative of the client device key in the authentication message.

Obtaining the authentication message may comprise including an authentication type identifier in the authentication message.

Advantageously, the method and intraoral scanning device enables the intraoral scanning device manufacturer to control client device access to the intraoral scanning device and/or enable version control in client device access.

The method and apparatus as disclosed provide a scalable security architecture for intraoral scanning device systems with improved security. The disclosed client device and method support an intraoral scanning device in combatting attacks such as unauthorized access or control of an intraoral scanning device, while still allowing access to legitimate parties such as the client device, for e.g., customization purposes, update purposes, maintenance purposes. The client device and method allow the intraoral scanning device to open a session only with authenticated parties, such as an authenticated customization device, an authenticated accessory device, an authenticated external device and/or an authenticated server. This may provide robustness against impersonation and masquerading attacks, battery exhaustion attacks, man-in-the-middle attacks and/or replay attacks. Further, the need for updating and/or exchange of keys in case a key has been compromised at a client device has been reduced and simplified.

The processing unit may be configured to process the received 2D image data and/or 3D image data for the purpose of visualizing the data on a display, for designing dental accessories, such as aligners, retainers, crowns, implants, bracers, nightguards etc., and/or for providing diagnostic data.

The 2D image data and/or the 3D image data may be image data configured to be visualizable on a display in a 2D or a 3D manner, respectively.

The term “client device” as used herein refers to a device that communicates with the intraoral scanning device. The client device may refer to a computing device acting as a client. The client device may comprise a customization device, a handheld device, a relay, a tablet, a personal computer, a mobile phone, and/or USB dongle plugged into a personal computer. The client device may control operation of the intraoral scanning device, either by sending customization data, intraoral scanning device operating parameters, and/or firmware data. The disclosed client device and method support the intraoral scanning device in combatting attacks such as unauthorized access or control of an intraoral scanning device, while still allowing access to legitimate parties such as the client device, for e.g., customization purposes, update purposes, maintenance purposes.

The intraoral scanning device may be operated in one or more modes. The one or more modes may include a first mode and/or a second mode. The one or more modes may include a third mode and/or a fourth mode. The one or more modes may include a default mode.

The client device may comprise a memory unit and a wireless interface respectively connected to the processing unit. The wireless interface may comprise a wireless transceiver, e.g., configured for wireless communication at frequencies in the range from 2.4 to 2.5 GHz, 2.4 GHz to 5 GHZ, about 2.45 GHz or about 5 GHz. The wireless transceiver may be a Bluetooth transceiver, a Bluetooth Low Energy transceiver, or a Wireless Fidelity (WIFI) transceiver. The wireless interface may form a connection to one or more other devices such as a computer, and/or a scan computer, and/or a tablet and/or a smart phone.

In an embodiment, the wireless interface is configured for communication, such as wireless communication, with an intraoral scanning device comprising a wireless transceiver.

The processing unit may be configured to send a session request for a session to the intraoral scanning device via the wireless interface. The processing unit may be configured to receive a session response from the intraoral scanning device via the wireless interface, e.g., from an intraoral scanning device and/or a session key apparatus. The session response may comprise the intraoral scanning device identifier or an identifier derived therefrom. In an exemplary client device, the client device may receive the intraoral scanning device identifier during a pairing of the client device and the intraoral scanning device. Hence, the processing unit comprises e.g., a receive/send unit configured to send data such as the session request and/or receive data such as the session response via the wireless interface. The processing unit may be configured to obtain a session key based on e.g., the session response, such as to extract the session key from or based on the session response. Hence, the processing unit comprises an obtainer. The processing unit may retrieve the session key from a key depository, e.g., stored in the memory unit. The processing unit may be configured to obtain a session key, wherein to obtain a session key may comprise to establish a connection to a session key apparatus via the wireless interface. The processing unit may send a session key request to the session key apparatus such as a session key server via the wireless interface e.g., via a wireless communication link established between the client device and the session key apparatus via the wireless interface. The processing unit may receive a session key response from the session key apparatus via the wireless interface and may determine the session key based on the session key response.

The session response may comprise an intraoral scanning device identifier. The intraoral scanning device identifier may comprise a hardware number of the intraoral scanning device and/or a serial number of the intraoral scanning device. The client device may retrieve the session key from the session key apparatus by providing the intraoral scanning device identifier to the session key apparatus, e.g., as part of the session key request, and requesting the session key or an intraoral scanning device key from the session key apparatus and/or requesting the session key apparatus to decrypt the session response and/or the session key.

In one or more exemplary client devices, the processing unit configured to obtain the session key may be configured to establish a connection to a session key apparatus via the wireless interface, to send a session key request to the session key apparatus via the wireless interface, to receive a session key response from the session key apparatus via the wireless interface, and to determine the session key based on the session key response. The session key request may comprise the intraoral scanning device identifier. The connection to the session key apparatus may be a secure connection over a network, such as including a private and/or a public network.

The session key apparatus may be a customization accessory device: wherein the customization accessory device optionally comprises a storage device containing a list configured to provide a session key and/or a session key response based on a session key request.

The processing unit may be configured to determine intraoral scanning device data. Hence the processing comprises e.g., a determiner. The intraoral scanning device data comprises e.g., firmware, customization data, and/or intraoral scanning device operating parameters. Customization data may for example be setting data of the intraoral scanning device, such as power management settings, configuration settings, configuration of a user interface of the intraoral scanning device and/or settings of an optical unit of the intraoral scanning device. Firmware may refer to a computer program provided by the intraoral scanning device manufacturer, and to be installed on the intraoral scanning device to control the intraoral scanning device. Firmware is for example to be installed to upgrade the operations and capabilities of the intraoral scanning device.

The optical unit may include one or more light projectors, one or more optical components, and one or more image sensors.

The user interface of the intraoral scanning device may include at least a touch sensor, at least a touch button, at least a light emitting diode, a haptic sensor, and/or an accelerometer.

The session response may comprise an encrypted session key. The processing unit may be configured to determine the session key by retrieving the session key from the session key response.

In one or more exemplary client device, to determine the session key comprises retrieving an intraoral scanning device key from the session key response or from the memory unit and decrypting the encrypted session key based on the intraoral scanning device key. To determine the session key may comprise decrypting the encrypted session key with a global key. A global is e.g., a key common to a group of client devices. The processing unit may be configured to retrieve an intraoral scanning device key from the session key response and decrypt the encrypted session key based on the intraoral scanning device key. The processing unit may comprise a decrypt/encrypt unit. The intraoral scanning device key may be e.g., a symmetric key or a public key of a private-public key pair. The intraoral scanning device key may comprise an AES-128 bits key as a symmetric key. The use of a symmetric key as an intraoral scanning device key provides the advantage of being able to use hardware accelerators. The intraoral scanning device key may comprise a public key of a private-public key pair, such as a public key of a private-public key pair of an authorized discloser of the session key, such as of the client device or the session key apparatus.

The processing unit may be configured to determine the session key by including a decryption of the encrypted session key with a global key, i.e., to determine the session key may comprise decrypting the encrypted session key with a global key. The global key may be e.g., a symmetric key or a public key of a private-public key pair. The session key may be compliant with an encryption standard such as Advanced Encryption Standard, AES, RSA crypto-system, Triple Data Encryption Algorithm.

The processing unit may be configured to generate session data, e.g., including a message authentication code, based on the session key and the intraoral scanning device data. Hence the processing unit may comprise a generator. The processing unit may generate a message authentication code based on the session key and the intraoral scanning device data. The message authentication code may be included in the session data. The processing unit may be configured to generate session data based on an intraoral scanning device key. The processing unit may be configured to digitally sign the intraoral scanning device data, such as to digitally sign the intraoral scanning device data using a private key of the client device, and/or of a group of client devices. The processing unit may be configured to digitally sign the intraoral scanning device data using a private key obtained from the session key apparatus, e.g., as part of a session key response. The processing unit may generate a digital signature using a signature generation function and a private key of a client device and append the digital signature to the session data. The intraoral scanning device may then verify the digital signature when receiving the session data. If the digital signature is not successfully verified using the alleged public key of a client device, the intraoral scanning device may disregard the session data and/or terminate the session. This may provide the advantage that the client device supports the intraoral scanning device in rejecting session data tampered or received from unauthenticated parties and the communication with the intraoral scanning device may thus be robust against impersonation and masquerading attacks.

The processing unit may be configured to send the session data to the intraoral scanning device via the wireless interface, e.g., using the receive/send unit. The session data may comprise intraoral scanning device data encrypted with the session key. To encrypt session data with the session key, the client device may utilize any of the above encryption standards.

The present disclosure relates to improved security in intraoral scanning device communication.—Namely, the client device disclosed herein enables intraoral scanning device communication that is robust against security threats, vulnerabilities, and attacks by implementing appropriate safeguards and countermeasures, such as security mechanisms, to protect against threats and attacks. The present disclosure relates to intraoral scanning device communication that is robust against replay attacks, unauthorized access, battery exhaustion attacks, and man-in-the-middle attacks.

As used herein, the term “intraoral scanning device” refers to a device configured to acquire intraoral scan data from a three-dimensional dental object during a scanning session.

As used herein, the term “certificate” refers to a data structure that enables verification of its origin and content, such as verifying the legitimacy and/or authenticity of its origin and content. The certificate is configured to provide a content that is associated to a holder of the certificate by an issuer of the certificate. The certificate comprises a digital signature, so that a recipient of the certificate is able to verify or authenticate the certificate content and origin. The certificate may comprise one or more identifiers and/or keying material, such as one or more cryptographic keys (e.g., an intraoral scanning device key) enabling secure communication in an intraoral scanning device system. The certificate permits thus to achieve authentication of origin and content, non-repudiation, and/or integrity protection. The certificate may further comprise a validity period, one or more algorithm parameters, and/or an issuer. A certificate may comprise a digital certificate, a public key certificate, an attribute certificate, and/or an authorization certificate. Examples of certificates are X.509 certificates, and Secure/Multipurpose Internet Mail Extensions, S/MIME, certificates, and/or Transport Layer Security, TLS, certificates.

As used herein, the term “key” refers to a cryptographic key, i.e., a piece of data, (e.g., a string, a parameter) that determines a functional output of a cryptographic algorithm. For example, during encryption, the key allows a transformation of a plaintext into a cipher-text and vice versa during decryption. The key may also be used to verify a digital signature and/or a message authentication code, MAC. A key is so called a symmetric key when the same key is used for both encryption and decryption. In asymmetric cryptography or public key cryptography, a keying material is a key pair, so called a private-public key pair comprising a public key and a private key. In an asymmetric or public key cryptosystem (such as Rivest Shamir Adelman, RSA, cryptosystem), the public key is used for encryption and/or signature verification while the private key is used for decryption and/or signature generation. The intraoral scanning device key may be keying material allowing derivation of one or more symmetric keys, such as a session key and/or a certificate key for intraoral scanning device communication. The intraoral scanning device key may be stored in a memory unit of the intraoral scanning device, e.g., during manufacture. The intraoral scanning device key may comprise keying material that is used to derive a symmetric key. The intraoral scanning device key comprises for example an Advanced Encryption Standard, AES, key, such as an AES-128 bits key.

As used herein the term “identifier” refers to a piece of data that is used for identifying, such as for categorizing, and/or uniquely identifying. The identifier may be in a form of a word, a number, a letter, a symbol, a list, an array, or any combination thereof. For example, the identifier as a number may be in the form of an integer, such as unsigned integer, unit, with a length of e.g., 8 bits, 16 bits, 32 bits, etc., such as an array of unsigned integers.

A client device for intraoral scanning device communication with an intraoral scanning device is disclosed. The term “client device” as used herein refers to a device that is able to communicate with the intraoral scanning device. The client device may refer to a computing device acting as a client. The client device may comprise a customization device, a handheld device, a relay, a tablet, a personal computer, an application running on a personal computer or tablet, or mobile phone and/or USB dongle plugged into a personal computer. The client device may be attributed a client device type indicated by a client device type identifier, the client device type e.g., corresponding to a model, category, or type of client devices, such as a customization type, e.g., a tablet product model, category or type for customizing the intraoral scanning device, a USB dongle product model, category or type for customizing the intraoral scanning device. The client device may be configured to control operation of the intraoral scanning device, either by sending customization data, intraoral scanning device operating parameters, and/or firmware data.

The client device comprises a memory unit and a wireless interface respectively connected to the processing unit. The memory unit may include removable and non-removable data storage units including, but not limited to, Read Only Memory (ROM), Random Access Memory (RAM), etc. The memory unit has a client device certificate stored thereon. The memory unit may have the client device certificate and/or the client device key stored at a memory address of the memory unit, and/or in memory cells of the memory unit, such as in designated memory cells and/or at designated addresses. The wireless interface may comprise a wireless transceiver, e.g., configured for wireless communication at frequencies in the range from 2.4 to 2.5 GHZ. The wireless interface may comprise one or more connectors for connection to another device, e.g., a customization device. A connector may be a standard connector, such as a USB connector (USB 2.0 standard-A, USB 2.0 standard-B, Micro-A USB, Micro-B USB, Mini-A USB, Mini-B USB, or others). A connector may be a proprietary connector used by a manufacturer of personal electronic devices. The wireless interface may be configured for communication, such as wireless communication, with an intraoral scanning device comprising a wireless transceiver. The client device certificate may comprise a certificate type identifier. The certificate type identifier may indicate a type of the certificate amongst a variety of certificate types, such as an intraoral scanning device family certificate type, an intraoral scanning device certificate type, a firmware certificate type, a research, and development certificate type, and/or a client device certificate type. The certificate type identifier may be used by an intraoral scanning device and/or the client device to identify what type of certificate an intraoral scanning device receives, stores, authenticates and/or retrieves. The client device certificate may comprise a version identifier indicative of a data format version of the certificate. An intraoral scanning device may use the certificate type identifier and/or the version identifier of the client device certificate to determine what type of data the client device certificate comprises and/or what type of data is comprised in a field of the client device certificate. For example, an intraoral scanning device may determine based on the certificate type identifier and/or version identifier what field of the client device certificate comprises a digital signature and/or which public key is needed to verify the digital signature of the client device certificate. It may be envisaged that there is a one-to-one mapping between the certificate type identifier and the public-private key pair.

The client device certificate may comprise a signing device identifier. The signing device identifier refers to a unique identifier identifying the device that has signed the client device certificate, such as a manufacturing device, e.g., an integrated circuit card, a smart card, a hardware security module. The signing device identifier may for example comprise a medium access control, MAC, address of the signing device and/or a serial number of the signing device. The signing device identifier may allow for example an intraoral scanning device to determine whether the signing device is e.g., black-listed or not, and thus to reject certificates signed by a signing device that has been black-listed, e.g., due to theft or other corruption.

The client device certificate may comprise a client device type identifier. The client device type identifier may indicate a type of the client device amongst a variety of client device types, such as a model, category or type of client devices, a USB dongle product model, category, or type for customizing the intraoral scanning device. The client device type identifier may be used by an intraoral scanning device to identify what type of client device the intraoral scanning device communicates with. The client device type identifier may enable an intraoral scanning device to select a set of keys from a plurality of key sets in the intraoral scanning device. Respective key sets in the intraoral scanning device may be used by respective different types of client devices.

The client device certificate may comprise a client device identifier. The client device identifier may be based on one or more hardware identifiers of one or more hardware components/modules of the client device.

The client device certificate may comprise a client device key identifier. The client device key identifier is indicative of the client device key.

The client device certificate may comprise one or more hardware identifiers, for example a first hardware identifier and/or a second hardware identifier. A hardware identifier may identify a piece of hardware comprised in the client device, such as a radio chip comprised in the client device or a digital signal processor of the client device. The hardware identifier(s) may be stored in a register of the piece of hardware comprised in the client device during manufacturing of the piece of hardware. The hardware identifier may comprise a serial number of the hardware, a chip identifier, or any combination thereof. The client device receiving or retrieving from the memory unit the client device certificate comprising the hardware identifier may verify the client device certificate by comparing its stored hardware identifier and the corresponding hardware identifier comprised in the client device certificate. Such verification may be performed upon retrieval of the client device certificate from the memory unit, such as at boot or power-on of the client device. The client device certificate may comprise one or more Bluetooth addresses, e.g., assigned by the manufacturer during manufacture.

The client device certificate may comprise a user identifier, e.g., in the form of a username. A client device certificate with a user identifier may facilitate the use of a generic device, such as a tablet computer, as a client device, e.g., by implementing a user verification/key generation/encryption at a remote server device, e.g., controlled by an intraoral scanning device manufacturer.

The client device certificate may comprise a digital signature. The digital signature enables a proof or verification of authenticity of the client device certificate, such as verification of the signer legitimacy. The digital signature is optionally generated by a manufacturing device using a client device family private key at manufacturing of the client device. The digital signature is verifiable by an intraoral scanning device and/or customization device using a corresponding client device family public key. If the digital signature is not successfully verified using the alleged public key, an intraoral scanning device may disregard the client device certificate and/or abort normal operation. This may provide the advantage that the intraoral scanning device rejects a client device certificate that is tampered or received from unauthenticated parties. The communication with the intraoral scanning device may thus be robust against impersonation, modification, and masquerading attacks.

The processing unit is configured to receive a connection response comprising an intraoral scanning device identifier via the wireless interface. The connection response may be generated by and/or sent from an intraoral scanning device. The processing unit is configured to generate one or more keys, e.g., based on the intraoral scanning device identifier and/or the client device key. To generate one or more keys may comprise to generate a common secret based on the client device key. To generate one or more keys may comprise to generate an intraoral scanning device key based on the intraoral scanning device identifier and/or the client device key, e.g., including to perform a hash function. For example, the intraoral scanning device key, IOS_KEY, may be given as:

$$IOS\_KEY = hash ( IOS\_ID , CD\_KEY ) .$$

where hash is a hash function, IOS_ID is the intraoral scanning device identifier and CD_KEY is the client device key.

By generating and/or using a common secret, a need for exchanging keys is avoided. Further, if the common secret is based on the intraoral scanning device identifier (client device key is different from intraoral scanning device key), the client device key cannot be derived from the intraoral scanning device key used by the intraoral scanning device. Thereby the risk of compromising the client device key is heavily reduced.

The one or more keys generated based on the intraoral scanning device identifier and/or the client device key may be based on the common secret.

The certificate key may be based on the common secret and/or a certificate value. The certificate value may be a predefined value or string, such as “certificate”. The certificate key may be generated by performing a hash function on the common secret and/or the certificate value. For example, the certificate key, C_KEY, may be given as:

$$C\_KEY = hash ( CS , C\_VAL ) ,$$

where hash is a hash function, CS is the common secret and C_VAL is the certificate value.

To generate one or more keys may comprise to generate a session key. The session may be different from the certificate key. The session key may be based on the intraoral scanning device identifier. The session key may be based on the session identifier. The session key may be based on the client device key. The processing unit may be configured to transmit the session key to a customization device. The client device, when configured to operate as a customization device, may be configured to perform customization communication with the intraoral scanning device based on the session key. The session key may be based on the common secret and/or a session value. The session value may be a predefined value or string, such as “session”. The session key may be generated by performing a hash function on the common secret and/or the session value. For example, the session key, S_KEY, may be given as:

$$S\_KEY = hash ( CS , S\_VAL ) ,$$

where hash is a hash function, CS is the common secret and S_VAL is the session value. By generating a session key based on a session identifier and a common secret, session specific communication is enabled.

The processing unit is configured to obtain an authentication message based on the certificate key and/or the client device certificate. To obtain the authentication message may comprise to include the client device certificate in the authentication message.

The processing unit may be configured to include an authentication key identifier, e.g., indicative of the client device key in the authentication message. The authentication key identifier may be indicative of or match the client device key identifier of the client device certificate. An authentication message comprising an authentication key identifier indicative of the client device key enables an intraoral scanning device to select a correct intraoral scanning device key from a plurality of intraoral scanning device keys, e.g., in order to generate or select the common secret. Subsequently, the intraoral scanning device may generate the certificate key for decrypting the encrypted client device certificate in the intraoral scanning device.

The processing unit may be configured to include an authentication type identifier in the authentication message. The authentication type identifier may be indicative of or match the client device type identifier and/or the certificate type identifier of the client device certificate. An authentication message comprising an authentication type identifier may enable an intraoral scanning device to select an intraoral scanning device key from a selected set of intraoral scanning device keys when the intraoral scanning device comprises a plurality of intraoral scanning device key sets. In addition, or alternatively, the intraoral scanning device may be configured to process the authentication message in different ways based on the authentication type identifier. Thus, an intraoral scanning device may be able to select an appropriate authentication message processing scheme.

The method comprises receiving a connection response, e.g., from an intraoral scanning device, via the wireless interface. The connection response may comprise an intraoral scanning device identifier. The method comprises generating and/or obtaining one or more keys, e.g., based on the intraoral scanning device identifier and/or the client device key. Generating one or more keys may comprise to generate a common secret based on the client device key. The one or more keys may comprise a certificate key.

The method comprises generating and/or obtaining an authentication message based on the certificate key and/or the client device certificate. Obtaining and/or generating the authentication message may comprise generating an encrypted client device certificate with the client device by encrypting the client device certificate with the certificate key and optionally including the encrypted client device certificate in the authentication message. Obtaining and/or generating the authentication message may comprise obtaining an encrypted client device certificate, e.g., from the memory unit and/or a server device. Obtaining an encrypted client device certificate may comprise transmitting a certificate request to a server device. In response, the server device generates and transmits a certificate response comprising the encrypted client device certificate (the client device certificate has been encrypted with certificate key). The client device receives the certificate response with the encrypted client device certificate and includes the encrypted client device certificate in the authentication message. Thus, obtaining an encrypted client device certificate may comprise receiving a certificate response comprising the encrypted client device certificate from a server device. Obtaining the authentication message may comprise including the client device certificate in the authentication message.

The method comprises transmitting the authentication message, e.g., to the intraoral scanning device, via the wireless interface.

The method may comprise obtaining a session identifier, e.g., by receiving the session identifier from the intraoral scanning device. The connection response may comprise the session identifier. Generating one or more keys may comprise generating an intraoral scanning device key based on the intraoral scanning device identifier and the client device key. For example, the intraoral scanning device key, IOS_KEY, may be given as:

$$IOS\_KEY = hash ( IOS\_ID , CD\_KEY ) ,$$

where hash is a hash function, IOS_ID is the intraoral scanning device identifier and CD_KEY is the client device key.

Generating one or more keys may comprise generating a common secret. The common secret may be based on the intraoral scanning device key and/or the session identifier. The common secret may be based on the intraoral scanning device identifier. The intraoral scanning device key and/or the client device key may be used as a common secret. For example, the common secret, CS, may be given as:

$$CS = hash ( IOS\_KEY , S\_ID ) ,$$

where hash is a hash function, IOS_KEY is the intraoral scanning device key and S_ID is the session identifier. Generating one or more keys may comprise generating one or more keys based on the common secret.

In the method, the certificate key may be based on the common secret and/or a certificate value. The certificate value may be a predefined value or string, such as “certificate”. Generating the certificate key may comprise performing a hash function on the common secret and/or the certificate value. For example, the certificate key, C_KEY, may be given as:

$$C\_KEY = hash ( CS , C\_VAL ) ,$$

where hash is a hash function, CS is the common secret and C_VAL is the certificate value.

Generating one or more keys may comprise generating a session key. The session key may be different from the certificate key. The session key may be based on the intraoral scanning device identifier. The session key may be based on the session identifier. The session key may be based on the client device key. The method may comprise transmitting the session key to a customization device. The method may comprise performing customization communication with the intraoral scanning device based on the session key.

In the method, the session key may be based on the common secret and/or a session value. The session value may be a predefined value or string, such as “session”. Generating the session key may comprise performing a hash function on the common secret and/or the session value. For example, the session key, S_KEY, may be given as:

$$S\_KEY = hash ( CS , S\_VAL ) ,$$

where hash is a hash function, CS is the common secret and S_VAL is the session value. By generating a session key based on a session identifier and a common secret, session specific communication is enabled.

Communication with an intraoral scanning device based on a common secret unique for the intraoral scanning device (e.g., common secret is based on intraoral scanning device identifier and/or session identifier) provides intraoral scanning device-specific communication. Thereby other intraoral scanning devices are not able to process/understand authentication messages intended for a specific intraoral scanning device.

In the method, generating the authentication message may comprise including an authentication key identifier in the authentication message. The authentication key identifier may be indicative of or match the client device key identifier of the client device certificate. An authentication message comprising an authentication key identifier indicative of the client device key enables an intraoral scanning device to select a correct intraoral scanning device key from a plurality of intraoral scanning device keys, e.g., in order to generate or select the common secret. Subsequently, the intraoral scanning device may generate the certificate key for decrypting the encrypted client device certificate in the intraoral scanning device, e.g., based on the selected intraoral scanning device key.

In the method, generating the authentication message may comprise including an authentication type identifier in the authentication message. The authentication type identifier may be indicative of or match the client device type identifier and/or the certificate type identifier of the client device certificate. An authentication message comprising an authentication type identifier may enable an intraoral scanning device to select an intraoral scanning device key from a selected set of intraoral scanning device keys when the intraoral scanning device comprises a plurality of intraoral scanning device key sets. In addition, or alternatively, the intraoral scanning device may be configured to process the authentication message in different ways based on the authentication type identifier. Thus, an intraoral scanning device may be able to select an appropriate authentication message processing scheme based on the authentication type identifier. The authentication type identifier may be the client device type identifier of the client device certificate.

In an exemplary method or an exemplary client device, the common secret, CS, may be given as:

$$CS = hash ( CD\_KEY , S\_ID ) ,$$

where hash is a hash function, CD_KEY is the client device key and S_ID is the session identifier.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 illustrates an exemplary architecture according to this disclosure;

FIG. 2 illustrates an exemplary intraoral scanning device:

FIG. 3 shows an exemplary sequence diagram between an intraoral scanning device and a client device:

FIG. 4 shows an exemplary sequence diagram:

FIG. 5 illustrates an exemplary flowchart of a method:

FIG. 6 illustrates an exemplary architecture according to this disclosure:

FIG. 7 illustrates an exemplary intraoral scanning device:

FIG. 8 shows an exemplary sequence diagram between an intraoral scanning device and a client device:

FIG. 9 shows an exemplary sequence diagram:

FIG. 10 illustrates an exemplary flowchart of a method:

FIG. 11 illustrates a system including an intraoral scanning device;

FIG. 12 illustrates an exemplary intraoral scanning device;

FIG. 13A shows examples of client device certificate key:

FIG. 13B illustrates an exemplary intraoral scanning device certificate:

FIG. 14 illustrates an exemplary sequence diagram:

FIG. 15 illustrates an exemplary flowchart of a method; and

FIG. 16 schematically illustrates a dental system:

FIG. 17 shows an exemplary signaling diagram:

FIG. 18 is a flow diagram of an exemplary method according to the invention, and

FIG. 19 schematically illustrates an exemplary server device.

FIG. 20 illustrates an exemplary architecture according to this disclosure:

FIG. 21A illustrates an exemplary intraoral scanning device:

FIG. 21B illustrates an exemplary client device;

FIG. 22 shows an exemplary sequence diagram:

FIG. 23 illustrates an exemplary flowchart of a method:

FIG. 24 is an illustration of intraoral scanning device service access by various clients according to the prior art:

FIG. 25 is an illustration like FIG. 20, wherein, however, a client specific intraoral scanning device service access control is implemented;

FIG. 26 is a block diagram of an intraoral scanning device wirelessly connected with an external device:

FIG. 27 is an example of a message sequence chart, wherein the user of an intraoral scanning device grants authorization to an intraoral scanning device to intraoral scanning device services by a gesture to the intraoral scanning device:

FIG. 28 shows a variation of the message sequence chart of FIG. 27:

FIGS. 29 and 30 are message sequence charts, which are carried out subsequently, wherein authorization to access intraoral scanning device services is granted by an entity trusted by the intraoral scanning device:

FIG. 31 shows a message sequence chart wherein a client authenticates itself to the intraoral scanning device:

FIG. 32 shows a variant of the message sequence chart of FIG. 31:

FIG. 33 is an illustration of a hierarchical security classification of intraoral scanning device services:

FIG. 34 is an illustration of an assignment of security levels to authorization methods:

FIG. 35 Schematically illustrates an exemplary architecture according to this disclosure:

FIG. 36 Schematically illustrates an exemplary client device;

FIG. 37 Schematically illustrates an exemplary client device certificate:

FIG. 38 schematically illustrates an exemplary client device certificate:

FIG. 39 schematically illustrates an exemplary signaling diagram;

FIG. 40 schematically illustrates an exemplary signaling diagram:

FIG. 41 schematically illustrates an exemplary signaling diagram:

FIG. 42 schematically illustrates a flowchart of an exemplary method; and

FIG. 43 schematically illustrates an exemplary signaling diagram.

### DETAILED DESCRIPTION

The detailed description set forth below in connection with the appended drawings is intended as a description of various configurations. The detailed description includes specific details for the purpose of providing a thorough understanding of various concepts.

However, it will be apparent to those skilled in the art that these concepts may be practiced without these specific details. Several aspects of the devices, systems, mediums, programs, and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

A scanning for providing intraoral scan data may be performed by a dental scanning system that may include an intraoral scanning device such as the TRIOS series scanners from 3Shape A/S. The dental scanning system may include a wireless capability as provided by a wireless network unit. The intraoral scanning device may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the intraoral scanning device is operated by projecting a pattern and translating a focus plane along an optical axis of the intraoral scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e., for a given arrangement of the scanning system relative to the object. After moving the intraoral scanning device relative to the object or imaging the object at a different view, a new stack of 2D images for that view may be captured. The focus plane position may be varied by means of at least one focus element, e.g., a moving focus lens. The intraoral scanning device is generally moved and angled during a scanning session, such that at least some sets of sub-scans overlap at least partially, in order to enable stitching in the post-processing. The result of stitching is the digital 3D representation of a surface larger than that which can be captured by a single sub-scan, i.e., which is larger than the field of view of the 3D scanning device. Stitching, also known as registration, works by identifying overlapping regions of 3D surface in various sub-scans and transforming sub-scans to a common coordinate system such that the overlapping regions match, finally yielding the digital 3D model. An Iterative Closest Point (ICP) algorithm may be used for this purpose. Another example of an intraoral scanning device is a triangulation scanner, where a time varying pattern is projected onto the dental object and a sequence of images of the different pattern configurations are acquired by one or more cameras located at an angle relative to the projector unit.

The intraoral scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono- or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the intraoral scanning device comprises a light source configured to excite fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The intraoral scanning device preferably further comprises optical components for directing the light from the light source to the surface of the dental object. The specific arrangement of the optical components depends on whether the intraoral scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the Illumination of the dental object is directed, using optical components of the intraoral scanning device, towards the image sensor(s). The image sensor(s) are configured to generate a plurality of images based on the incoming light received from the illuminated dental object. The image sensor may be a high-speed image sensor such as an image sensor configured to acquire images with exposures of less than 1/1000 second or frame rates in excess of 250 frames pr. Second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The network unit may be configured to connect the dental scanning system to a network comprising a plurality of network elements including at least one network element configured to receive the processed data. The network unit may include a wireless network unit. The wireless network unit is configured to wirelessly connect the dental scanning system to the network comprising the plurality of network elements including the at least one network element configured to receive the processed data.

The dental scanning system preferably further comprises a processor configured to generate scan data (such as intraoral scan data) by processing the two-dimensional (2D) images acquired by the intraoral scanning device. The processor may be part of the intraoral scanning device. As an example, the processor may comprise a Field-programmable gate array (FPGA) and/or an Advanced RISC Machines (ARM) processor located on the intraoral scanning device. The scan data comprises information relating to the three-dimensional dental object. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g., described by image coordinates and a timestamp (x, y, t), wherein depth information can be inferred from the timestamp. The image sensor(s) of the intraoral scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g., described by image coordinates and a timestamp (x, y, t) or alternatively described as (x, y, z). The intraoral scanning device may be configured to transmit other types of data in addition to the scan data. Examples of data include 3D information, texture information such as infra-red (IR) images, fluorescence images, reflectance color images, x-ray images, and/or combinations thereof.

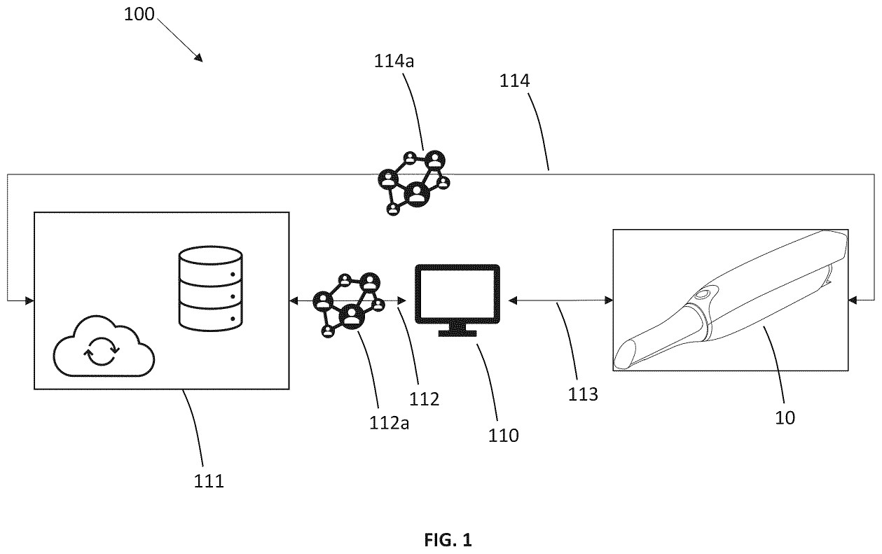

FIG. 1 illustrates an exemplary architecture 100 according to this disclosure. The architecture 100 comprises an intraoral scanning device 10, a client device 110, and a server device 111. The client device 110 may comprise a computing device acting as a client, a customization device, a handheld device, a relay, a tablet, a personal computer, a mobile phone, and/or USB dongle plugged into a personal computer. The server device 111 may comprise a computing device configured to act as a server, i.e., to serve requests from the client device 110 and/or from the intraoral scanning device 10. The server device 111 may be controlled by the intraoral scanning device manufacturer.

The intraoral scanning device 10 may be connected to the client device 110 via a communication link 113, such as a wireless communication link or a bidirectional wireless communication link. The wireless communication link may be carried over a short-range communication system, such as Bluetooth, Bluetooth low energy, IEEE 802.11, Zigbee, WIFI. The intraoral scanning device 10 may be connected to the client device 110 over a network.

The intraoral scanning device 10 may be connected to the server device 111 via a wireless communication link 114 or a bidirectional wireless communication link 114 over a network 114a, such as a bidirectional wireless communication link and/or wireless communication link over a network.

The client device 110 may be connected to the server device 111 via a communication link 112 over a network 112a, such as a bidirectional wireless communication link and/or wireless communication link over a network. In an embodiment, the network 112a may be the Internet.

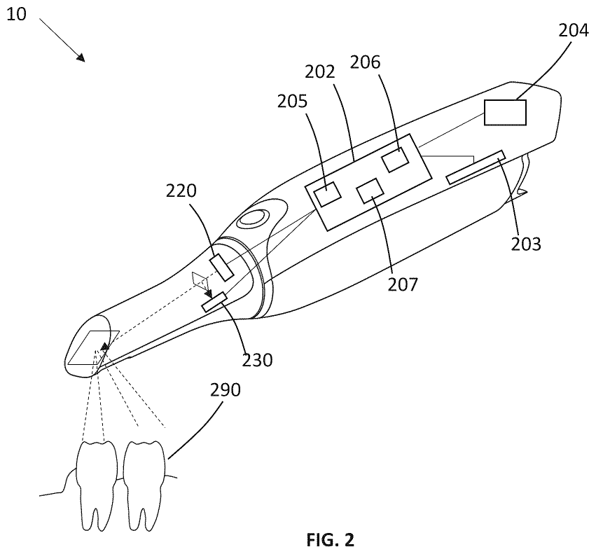

FIG. 2 illustrates an exemplary intraoral scanning device 10. The exemplary intraoral scanning device 10 comprises a processing unit 202 configured to process intraoral scan data of a patient 290 and provide 2D image data and/or 3D image data. The exemplary intraoral scanning device 10 comprises a memory and a wireless interface 204. The memory is in FIG. 2 illustrated in the form of a memory unit 203 external to the processing unit 202. The memory may in other exemplary intraoral scanning devices be at least partly embedded in the processing unit 202 and/or in the memory unit 203.

The processing unit 202 is configured to receive a mode request via the wireless interface 204. Hence, the processing unit 202 comprises a receive/send unit 205 configured to send and/or receive via the wireless interface 204. The receive/send unit 205 is configured to send and receive via the wireless interface 204 to/from an external device, such as a server device, a client device, a customization device, an accessory, a relay device, a smart phone. The processing unit 202 is configured to authenticate the mode request. Hence, the processing unit 202 may comprise an authenticator 206 configured to authenticate the mode request. The processing unit 202 is configured to place the intraoral scanning device into the requested mode, such as a service mode, a customization mode, an upgrade mode or debug mode, if authentication of the mode request succeeds. Hence the processing unit 202 comprises a mode controller 207 configured to place the intraoral scanning device 10 into the requested mode, e.g., based on an output from the authenticator 206. In the intraoral scanning device in FIG. 2, the processing unit 202 is configured to place the intraoral scanning device into a default mode if authentication of the mode request fails, the default mode comprising booting the intraoral scanning device and operating the intraoral scanning device according to operating parameters set during booting. In an embodiment, the operating parameters set during booting may be stored in a non-volatile part of the memory unit 203. In an embodiment, the operating parameters set during booting may comprise a default setting enabling the intraoral scanning device to function according to a default setting programmed during production of the intraoral scanning device.

The intraoral scanning device comprises a light projector 220 and an image sensor 230. The light projector includes at least one or more light emitting diodes and/or one or more infrared light source for emitting light pattern to a three-dimensional dental object 290 of a patient or of a wax model 290 which is a replicate of the patient's dental. The image sensor 230 receives the reflective light from the dental object 290, and the image sensor 230 converts the reflected light into intraoral scan data. The processing unit 202 is then configured to process the intraoral scan data to 2D image data and/or 3D image data. The image data is then forwarded to the wireless interface 204 which transmits the data to an external device.

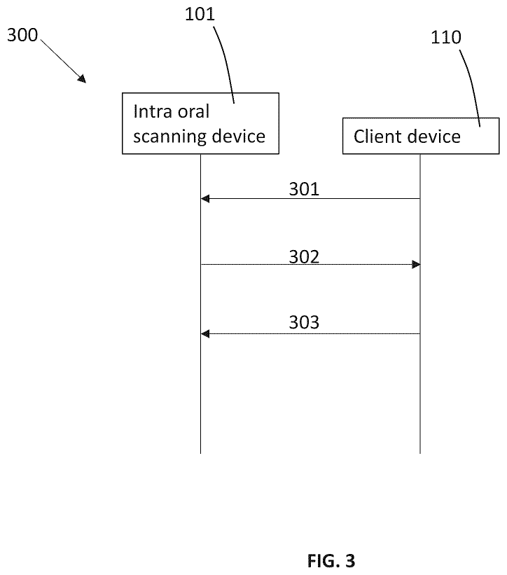

FIG. 3 shows an exemplary sequence diagram 300 between an intraoral scanning device 10 and a client device 110. In an embodiment, the client device 110 may be in the form of a customization device. The intraoral scanning device 10 receives a customization mode request 301 via the wireless interface 204 from the client device 110, the mode request comprising a digital signature and a mode identifier. The digital signature may be a signature according to the Digital Signature Standard or other suitable standards, such as RSA, for digital signatures known in the art. The intraoral scanning device 10 authenticates the mode request by verifying the digital signature. In the illustrated sequence diagram 300, the authentication succeeds, and the processing unit 202 places the intraoral scanning device 10 in the customization mode including sending a customization mode response 302 to the client device via the wireless interface 204. In the customization mode of the intraoral scanning device 10, a firmware part of the memory is write-protected, and a customized mode part of the memory is write-enabled.

Upon receipt of the customization mode response 302, the client device 110 sends data 303 to the intraoral scanning device 10 which receives the data and authenticates the received data 303, e.g., by use of digital signature or a session identifier/key as described earlier. If authentication of data 303 succeeds, the processing unit 202 derives intraoral scanning device data (customization data) from the data 303 and stores intraoral scanning device data (customization data) in a customization part of the memory. If authentication of data 303 fails, the processing unit 202 places the intraoral scanning device in default mode.

When the customization data have been transferred, the client device may send a mode exit request and the intraoral scanning device is configured to optionally authenticate the mode exit request and to place the intraoral scanning device in the default mode, optionally if authentication of the mode exit request succeeds.

In another embodiment, the client device may be in the form of a smart phone or a tablet and may comprise software configured to provide the functionality of a customization device.

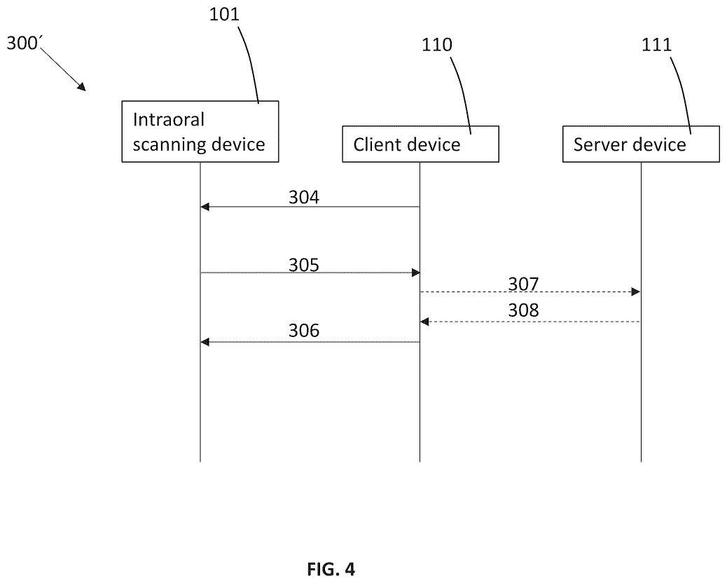

FIG. 4 shows an exemplary sequence diagram 300′ where a client device 110 is used for updating firmware of the intraoral scanning device 10, and a client device 110 in the form of a customization device. The customization device 10 receives a service mode request 304 via the wireless interface 204 from the client device 110. The intraoral scanning device 10 authenticates the service mode request. In the illustrated sequence diagram 300′, the authentication succeeds, and the processing unit 202 places the intraoral scanning device 10 in the service mode including sending a service mode response 305 to the client device via the wireless interface 204. In the service mode of intraoral scanning device 10, the processing unit 202 is allowed to write to a firmware part of the memory.

Upon receipt of the service mode response 305, the client device 110 sends data 306 to the intraoral scanning device 10 which receives the data and authenticates the received data 306, e.g., by use of digital signature or a session identifier/key as described earlier. Before sending data to the intraoral scanning device, the client device 110 may correspond with a server device 111 as illustrated with dotted arrows 307, 308, e.g., in order to determine the data 306 to be sent to the intraoral scanning device 10. If authentication of data 306 succeeds, the processing unit 202 derives intraoral scanning device data (firmware data) from the data 306 and stores intraoral scanning device data (firmware data) in a firmware part of the memory. If authentication of data 306 fails, the processing unit 202 may place the intraoral scanning device in default mode and/or terminate the session.

When the firmware has been transferred, the client device may send a mode exit request and the intraoral scanning device is configured to optionally authenticate the mode exit request and place the intraoral scanning device in the default mode, optionally if authentication of the mode exit request succeeds.

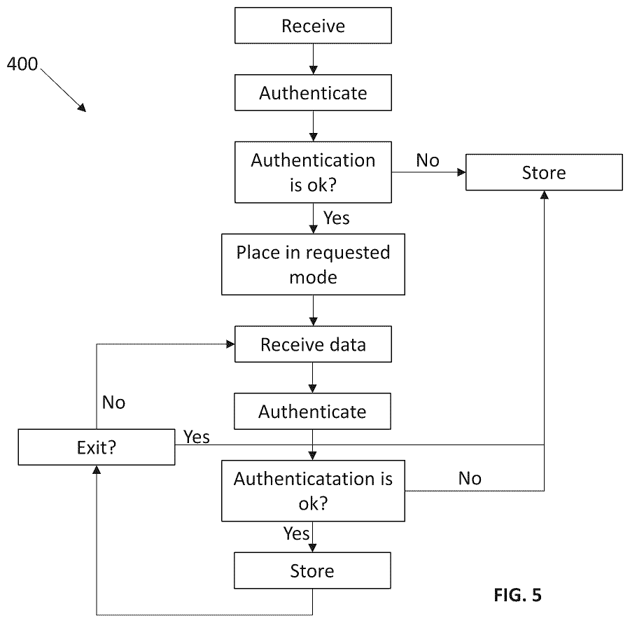

FIG. 5 illustrates an exemplary flowchart of a method 400, e.g., for configuration of a intraoral scanning device 10, comprising a processing unit 202 configured to process intraoral scan data of a patient and provide 2D image data and/or 3D image data, a memory, and a wireless interface 204. The method 400 comprises receiving 401 a mode request via the wireless interface 204 and authenticating 402 the mode request. Authenticating 402 the mode request comprises authenticating the sender of the mode request and verifying integrity of the mode request. If authentication of the mode request succeeds 404, the method proceeds to placing 403 the intraoral scanning device 10 into the requested mode. If authentication of the mode request fails 404, the method optionally proceeds to placing 405 the intraoral scanning device 10 into a default mode. After placing the intraoral scanning device 10 in the requested mode, the method optionally proceeds to receiving 408 data via the wireless interface, authenticating 410 the received data; and storing 412 intraoral device data in a part of the memory corresponding to the requested mode and based on the received data if authentication of the data succeeds. If authenticating 410 the received data fails, the method may proceed to placing 405 the intraoral scanning device in default mode or another mode and/or terminating the session. Upon storing, the method 400 optionally comprises to evaluate 414 whether a mode exit request has been received. If so, the method proceeds to placing 405 the intraoral scanning device in default mode. If not, the method proceeds to receiving 408 data.

Intraoral Scanning Device with Communication Protection and Related Method:

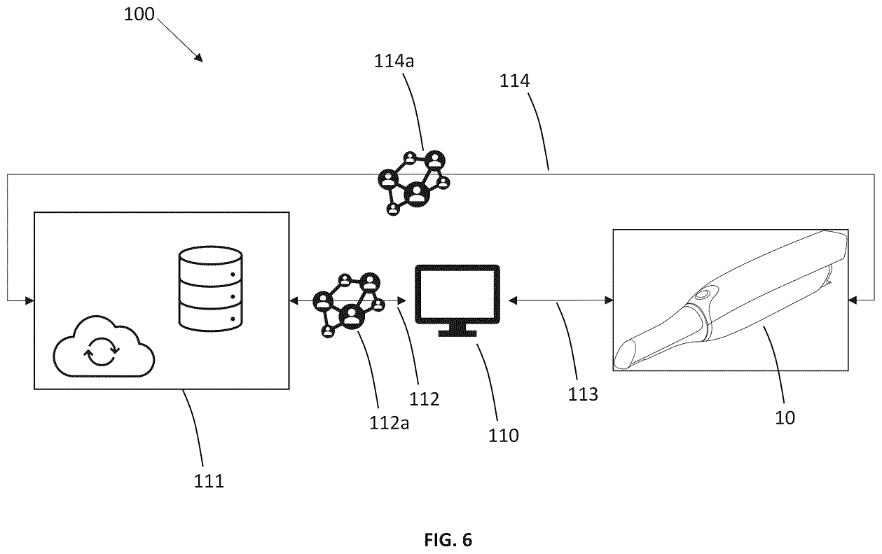

FIG. 6 schematically illustrates an exemplary architecture 100 according to this disclosure. The architecture 100 comprises an intraoral scanning device 10, a client device 110, and a server device 111. The client device 110 may comprise a computing device acting as a client, such as a customization device, a handheld device, a relay, a tablet, a personal computer, a mobile phone, and/or USB dongle plugged in a personal computer. The server device 111 may comprise a computing device configured to act as a server, i.e., to serve requests from the client device 110 and/or from the intraoral scanning device 10. The server device 111 may be controlled by the intraoral scanning device manufacturer.

The intraoral scanning device 10 may be connected to the client device 110 via a communication link 113, such as a bidirectional communication link and/or a wireless communication link. The wireless communication link may be carried over a short-range communication system, such as Bluetooth, Bluetooth low energy, and/or WIFI. The intraoral scanning device 10 may be connected to the client device 110 over a network.

The intraoral scanning device 10 may be connected to the server device 111 via a wireless communication link 114 or a bidirectional wireless communication link 114 over a network 114a, such as a bidirectional wireless communication link and/or wireless communication link over a network.

The client device 110 may be connected to the server device 111 via a communication link 112 over a network 112a, such as a bidirectional wireless communication link and/or wireless communication link over a network. In an embodiment, the network 112a may be the Internet.

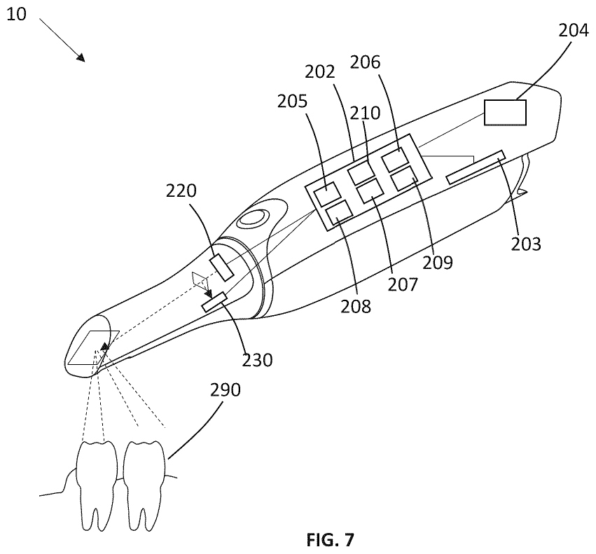

FIG. 7 schematically illustrates an exemplary intraoral scanning device 10. The exemplary intraoral scanning device 10 comprises a processing unit 202 configured to configured to process intraoral scan data of a patient and provide 2D image data and/or 3D image data.

The exemplary intraoral scanning device 10 comprises a memory unit 203 and a wireless interface 204 respectively connected to the processing unit 202. The memory is in FIG. 7 illustrated in the form of a memory unit 203 external to the processing unit 202. The memory may in other exemplary intraoral scanning devices be at least partly embedded in the processing unit 202 and/or in the memory unit 203. The processing unit 202 is configured to receive a session request for a session via the wireless interface 204. Hence, the processing unit 202 comprises a receive/send unit 205 configured to send and/or receive via the wireless interface 204. The receive/send unit 205 is configured to send and receive via the wireless interface 204 to/from an external device, such as a server device, a client device, a customization device, an accessory, a relay device, a tablet. The processing unit 202 is configured to obtain and store a session key. Hence, the processing unit 202 may comprise an obtainer 206 configured to obtain and/or generate the session key and to store the session key in e.g., the memory unit 203. To obtain the session key may comprise to generate a random or pseudo-random number with the number generator 210 contained in the intraoral scanning device 101. The processing unit 202 may comprise a random number generator 210. The session key obtained may be a 128-bits random or pseudo-random number, a 192-bits random or pseudo-random number, a 256-bits random or pseudo-random number, or any other bit-size random or pseudo-random number. The session key may be obtained so as to be compliant with an encryption standard such as Advanced Encryption Standard, AES, RSA crypto-system, Triple Data Encryption Algorithm, TDEA, Elliptic Curve Cryptographic system, any other encryption system. The session key may be uniquely generated for each session, using e.g., the number generator 210. This provides robustness against e.g., replay attacks.

The processing unit 202 is configured to sign the session key by an intraoral scanning device key, when the intraoral scanning device key may be stored in the permanent memory of the intraoral scanning device. The processing unit 202 is configured to send a session response comprising the signed session key, for example by using the receive/send unit 205 and the wireless interface 204. The processing unit 202 is configured to receive session data in the session via the wireless interface 204, for example by using the receive/send unit 205 and the wireless interface 204. The processing unit 202 receives the session data in the session from an external device, such as a client device 110 and/or a server device 111.

The processing unit 202 is configured to encrypt the session key based on an intraoral scanning device key. Hence, the processing unit 202 may comprise an encrypt/decrypt unit 207. The processing unit 202 is configured to send a session response comprising the encrypted session key which may be signed by the intraoral scanning device key, for example by using the receive/send unit 205 and the wireless interface 204. The processing unit 202 is configured to receive session data in the session via the wireless interface 204, for example by using the receive/send unit 205 and the wireless interface 204. The processing unit 202 receives the session data in the session from an external device, such as a client device 110 and/or a server device 111.

The intraoral scanning device comprises a light projector 220 and an image sensor 230. The light projector includes at least one or more light emitting diodes and/or one or more infrared light source for emitting light pattern to a three-dimensional dental object 290 of a patient or of a wax model 290 which is a replicate of the patient's dental. The image sensor 230 receives the reflective light from the dental object 290, and the image sensor 230 converts the reflected light into intraoral scan data. The processing unit 202 is then configured to process the intraoral scan data to 2D image data and/or 3D image data. The image data is then forwarded to the wireless interface 204 which transmits the data to an external device.

In one or more exemplary intraoral scanning devices, the processing unit 202 is configured to verify integrity of the session data. Thus, the processing unit 202 may comprise a verifier 208. For example, the processing unit 202 is configured to determine whether the integrity of the session data is corrupted, i.e., whether the session data has been tampered with or modified by an unauthorized party. The processing unit 202 detects e.g., an insertion, a deletion, and/or a substitution of data by an unauthorized party, such as by any party other than the sender.

In one or more exemplary intraoral scanning devices, the processing unit 202 is configured to terminate the session if integrity of the session data is corrupted. Thus, the processing unit 202 may comprise a terminator 209. For example, when it is determined by the processing unit 202 or the verifier 208 that the session data has been tampered with or modified (by e.g., insertion, deletion and/or substitution) by an unauthorized party, The processing unit 202 reject the received session data, and the terminator 209 or the processing unit 202 terminates the session with e.g. an external device. Terminating the session comprises deleting the session key from e.g., the memory unit 203. Deleting the session key protects the intraoral scanner device 10 against any replay attack or any attack based on any communication captured by an attacker.

In one or more exemplary intraoral scanning devices, the session data comprises a message authentication code, and the processing unit 202 verifying integrity of the session data is configured to verify the message authentication code with the stored session key. Thus, the verifier 208 is configured to verify the message authentication code with the stored session key. A message authentication code, MAC, is generated by a sender, such as an external device, based on the session data and the decrypted session key. Upon reception of the session data comprising the MAC, the intraoral scanning device which holds the stored session key is able to re-compute the MAC based on the received session data and a MAC generation function and compare the recomputed MAC with the received MAC. If the recomputed MAC does not match the received MAC, then the intraoral scanning device concludes that session data is corrupted. The intraoral scanning device disregards the session data and terminates the session. For example, the processing unit 202 disregards the session data and terminates the session using the terminator 208.

In one or more exemplary intraoral scanning devices, the session data comprises a digital signature. The processing unit 202 verifying integrity of the session data is configured to verify the digital signature. For example, the verifier 208 is configured to verify the digital signature. The processing unit 202 verifies the digital signature using a signature verification function and a public key of a sender that has generated the digital signature and appended it to the session data. If the processing unit 202 determines that the digital signature is not successfully verified using the alleged public key of a sender, the processing unit 202 disregards the session data and terminates the session. This may provide the advantage that the intraoral scanning device 10 rejects session data tampered or received from unauthenticated parties and is thus robust against impersonation and masquerading attacks.

In one or more exemplary intraoral scanning devices, the processing unit 202 is configured to decrypt the session data with the session key, and to store at least part of decrypted session data in the memory unit 203. A client device 110 or a server device 111 may send encrypted session data to the intraoral scanning device 10. The processing unit 202 receives the encrypted session data via the receive/send unit 205 and the wireless interface 204. The processing unit 202 retrieves the session key from e.g., the memory unit 203, and decrypts the session data using the retrieved session key and a decryption function. The processing unit 202 stores the decrypted session data in the memory unit 203. The processing unit 202 is configured to terminate the session if decryption of the session data fails. The decryption of the session data may fail if the session data is encrypted with a key different from the session key stored at the intraoral scanning device 10. This may provide the advantage that the intraoral scanning device 10 rejects session data received from parties not holding the session key and is thus robust against attacks based on such illegitimate data.

In one or more exemplary intraoral scanning devices, the session data comprises customization data, intraoral scanning device operating parameters, and/or firmware data. Firmware may refer to a computer program provided by the intraoral scanning device manufacturer, and to be installed on the intraoral scanning device to control the intraoral scanning device. Firmware is for example to be installed to upgrade the operations and capabilities of the intraoral scanning device. The customization data may include, for example, settings of a color image sensor of an intraoral scanning device. An intraoral scanning device may include a color image sensor, such as an RGB image sensor where the customization data may include information about different color areas to be deactivated and/or activated during at least a scanning session. Thus, the customization data may relate to which color areas of the RBG image sensor should be activated or deactivated during a scanning session. An intraoral scanning device may include a monochromatic image sensor and colored light emitting diodes, and in this example, the customization data may include information about which of the different colored light emitting diodes should be deactivated and/or activated during a scanning session. Thus, the customization data may include information that relates to which colored light emitting diodes should be activated or deactivated during a scanning session. A colored light emitting diode may be configured to emit light with a color, such as blue, red, green etc. In another example, the intraoral scanning device could include one or more near-infrared light emitting diodes which also can be set to be activated and/or deactivated during a scanning session by the customization data. The customization data may include setting data, such as power management settings, configuration of a user interface of the intraoral scanning device and/or settings of an optical unit of the intraoral scanning device

The optical unit may include one or more light projectors, one or more optical components, and one or more image sensors.

In one or more exemplary intraoral scanning devices, the processing unit 202 is configured to process intraoral scan data of a patient and provide 2D image data and/or 3D image data according to the received session data. The processing unit 202 may perform the process intraoral scan data of a patient and provide 2D image data and/or 3D image data based on the customization data.

In one or more exemplary intraoral scanning devices, the session key comprises a symmetric key. The symmetric key may be uniquely generated for each session, using e.g., the number generator 210. The symmetric key may comprise an AES-128 bits key. The use of a symmetric key as a session key may reduce a processing power requirement and allow the intraoral scanning device 10 to decrypt the session data with light encryption algorithms.

In one or more exemplary intraoral scanning devices, the intraoral scanning device key is a symmetric key or a public key of a private-public key pair. The intraoral scanning device key may comprise an AES-128 bits key as a symmetric key. The use of a symmetric as an intraoral scanning device key provides the advantage of being able to use hardware accelerators. The intraoral scanning device key may comprise a public key of a private-public key pair, such as a public key of a private-public key pair of an authorized discloser of the session key, such as of a server 111.

In one or more exemplary intraoral scanning devices, the processing unit 202 is configured to send an intraoral scanning device identifier in the session response. The intraoral scanning device identifier may refer to a unique device identifier. The intraoral scanning device identifier may comprise a hardware number, a serial number, a MAC address. The session response may comprise the encrypted session key and the intraoral scanning device identifier. The processing unit 202 is configured to send an intraoral scanning device identifier in the session response to an external device such as the client device 110 or to the server device 111. The intraoral scanning device key may be stored in a permanent memory, such as memory unit 203 of the intraoral scanning device 10 during production of the intraoral scanning device 10.

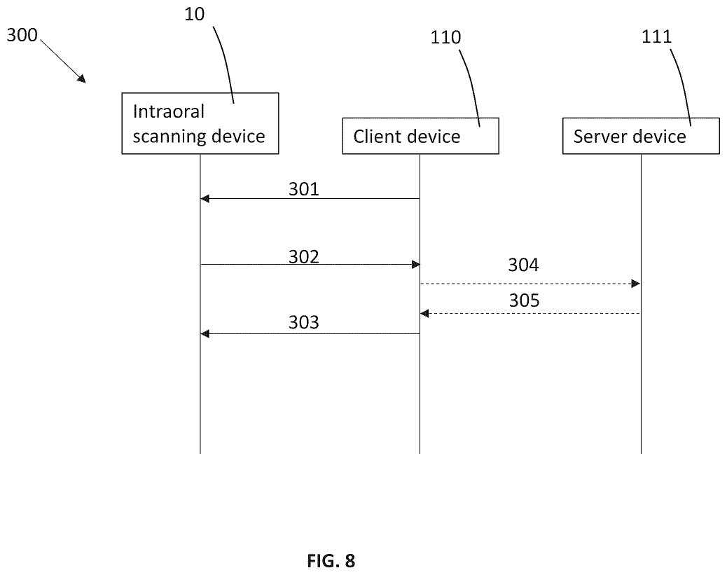

FIG. 8 shows an exemplary sequence diagram 300 between an intraoral scanning device 10, and a client device 110. The intraoral scanning device 10 receives a session request 301 for a session via the wireless interface 204 from the client device 110. The intraoral scanning device 10 obtains and stores a session key. The intraoral scanning device 10 encrypts the session key based on an intraoral scanning device key. The intraoral scanning device 10 sends to the client device 110 a session response 302 comprising the encrypted session key. The intraoral scanning device 10 may alternatively send to the client device 110 a session response 302 encrypted with the intraoral scanning device key, the session response 302 comprising the session key. The client device 110 receiving the session response 302 may request 304 the server device 111 to decrypt the encrypted session key comprised in the session response 302, or to decrypt the encrypted session response 302. Based on the request 304, the server device 111 may send the decrypted session key in a response 305 to the client device 110. This may be when the intraoral scanning device key used at the intraoral scanning device 10 is a public key of a private-public key pair of the server 101. When the intraoral scanning device key is a symmetric key, the server device 111 may send the decrypted session key in a response 305 to the client device 110 or send the intraoral scanning device key in the response 305 to the client device 110 which is then capable of decrypting the session key or the session response 302. The response 305 may comprise the decrypted session key or the intraoral scanning device key. The communication link 112 between the client device 110 and the server device 111 is secure, i.e., authenticated, encrypted and/or integrity protected using a security protocol (e.g. Transport Layer Security protocol). The intraoral scanning device 10 receives from the client device 110 session data 303 in the session via the wireless interface 204. The session data 303 may then be encrypted and/or integrity-protected at the client device 110, e.g., by use of the session key. In one or more exemplary intraoral scanning devices, the processing unit 202 is configured to verify integrity of the session data as described earlier in connection with FIG. 2. The session data 303 may comprise a message authentication code. A message authentication code, MAC, is generated by a sender, such as a client device/server device, based on the session data and the decrypted session key. Upon reception of the session data comprising the MAC, the intraoral scanning device which holds the stored session key is able to re-compute the MAC based on the received session data and a MAC generation function and compare the recomputed MAC with the received MAC. If the recomputed MAC does not match the received MAC, then the intraoral scanning device concludes that session data is corrupted. The intraoral scanning device disregards the session data and terminates the session. For example, the processing unit 202 disregards the session data and terminates the session using the terminator 208.

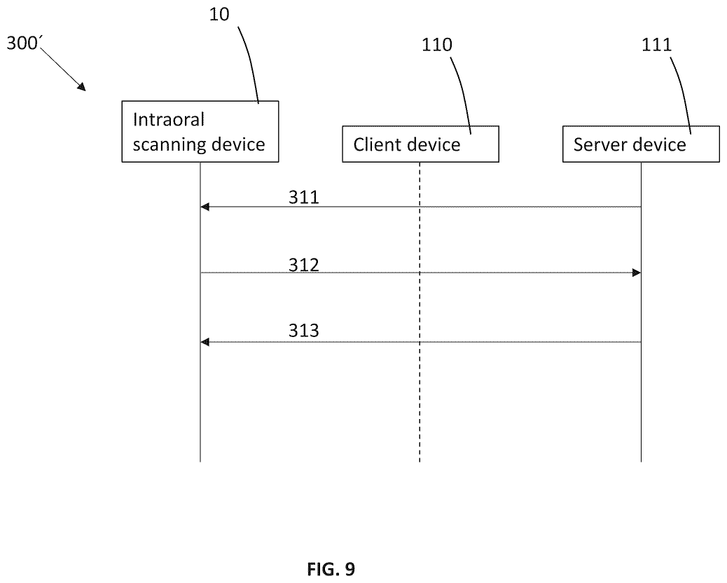

FIG. 9 shows an exemplary sequence diagram 300′ between an intraoral scanning device 10, a client device 110, and a server device 111. The intraoral scanning device 10 receives a session request 311 for a session via the wireless interface 204 from the server device 111. The intraoral scanning device 10 obtains and stores a session key. The intraoral scanning device 10 encrypts the session key based on an intraoral scanning device key. The intraoral scanning device 10 sends to the server device 111 a session response 312 comprising the encrypted session key. The intraoral scanning device 10 may alternatively send to the server device 111 a session response 312 encrypted with the intraoral scanning device key, the session response 312 comprising the session key. The server device 111 may decrypt the encrypted session key comprised in the session response 312, or the encrypted session response 312. The communication link 112 between the intraoral scanning device 10 and the server device 111 may be over the client device 110 and/or over a network. The communication link 112 between the intraoral scanning device 10 and the server device 111 may or may not be secure, i.e., authenticated, encrypted and/or integrity protected using a security protocol. The intraoral scanning device 10 receives from the server device 111 session data 313 in the session via the wireless interface 204. The session data 313 may then be encrypted and/or integrity-protected at the server device 111.

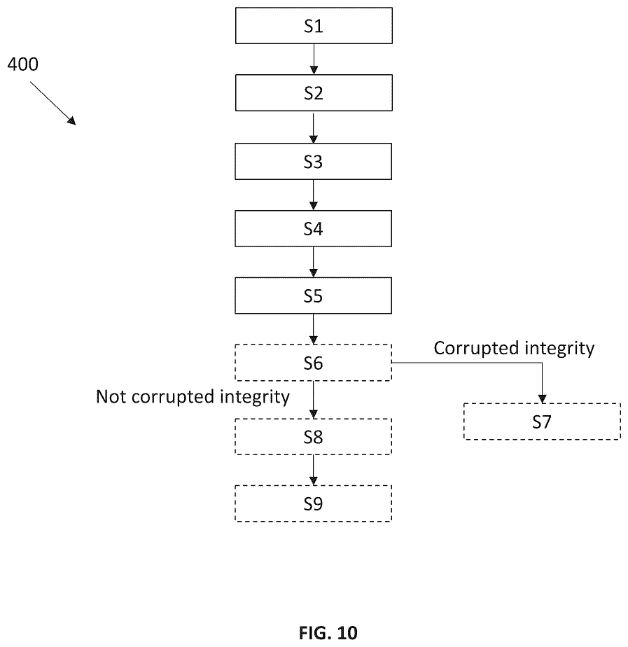

FIG. 10 schematically illustrates a flowchart 400 of an exemplary method according to this disclosure. The method 400 is proposed for communication of an intraoral scanning device 10 comprising a processing unit 202, a memory unit 203, and a wireless interface 204, such as for protecting communication of an intraoral scanning device with e.g., an external device (client device and/or server device and/or accessory equipment). The method 400 is performed in the intraoral scanning device 10. The method 400 comprises receiving S1 a session request for a session via the wireless interface 204. Receiving S1 may comprise receiving a session request from an external device, such as the client device 110 and/or the server device 111.

The method 400 comprises obtaining and storing S2 a session key, such as in a memory unit 203. Obtaining and storing S2 a session key, such as in a memory unit 203 may comprise generating a random or pseudo-random number with the number generator 210 contained in the intraoral scanning device 10. Obtaining and storing S2 a session key may comprise generating a 128-bits random or pseudo-random number, a 192-bits random or pseudo-random number, a 256-bits random or pseudo-random number, or any other bit-size random or pseudo-random number.

The method 400 comprises encrypting S3 the session key based on an intraoral scanning device key. Encrypting and/or signing S3 the session key based on an intraoral scanning device key. The encrypting may comprise using an encryption standard such as Advanced Encryption Standard, AES, RSA crypto-system, Triple Data Encryption Algorithm, TDEA, Elliptic Curve Cryptographic system, any other encryption system.

The method 400 comprises sending S4 a session response comprising the encrypted and/or signed session key and receiving S5 session data in the session via the wireless interface. Sending S4 a session response comprising the encrypted and/or signed session key may comprise sending the session response to a client device 110 and/or a server device 111. Receiving S5 session data in the session via the wireless interface 204 may comprise receiving session data from a client device 110 and/or a server device 111.

In one or more exemplary methods, the method 400 comprises verifying S6 integrity of the session data. Verifying S6 integrity of the session data may comprise determining whether the integrity of the session data is corrupted, i.e., determining whether the session data has been tampered with or modified by an unauthorized party. Verifying S6 integrity of the session data may comprise detecting e.g., an insertion, a deletion, and/or a substitution of data by an unauthorized party, such as by any party other than the legitimate sender. Verifying S6 integrity of the session data may comprise verifying a message authentication code appended to the session data (e.g., using the stored session key) and/or a digital signature appended to the session data. When it is determined that the integrity of the session data is not corrupted, then the intraoral scanning device 10 may grant access to reading and/or writing memory areas to the external device. For example, the session data or intraoral scanning device data derived therefrom may be written in the memory unit 203.

The method 400 comprises terminating S7 the session if integrity of the session data is corrupted. For example, if it is determined that the session data has been tampered with or modified (by e.g., insertion, deletion and/or substitution) by an unauthorized party, terminating S7 comprises rejecting the received session data, and terminating the session with e.g. an external device. Terminating S7 may comprise deleting the session key from e.g., the memory unit 203.

In one or more exemplary methods, the method 400 comprises decrypting S8 the session data with the session key and storing S9 at least part of decrypted session data in the memory unit. Decrypting S8 the session data with the session key may comprise retrieving the session key from e.g., the memory unit 203, and decrypts the session data using the retrieved session key and a decryption function.

### An Intraoral Scanning Device and Method of Intraoral Scanning Device Communication:

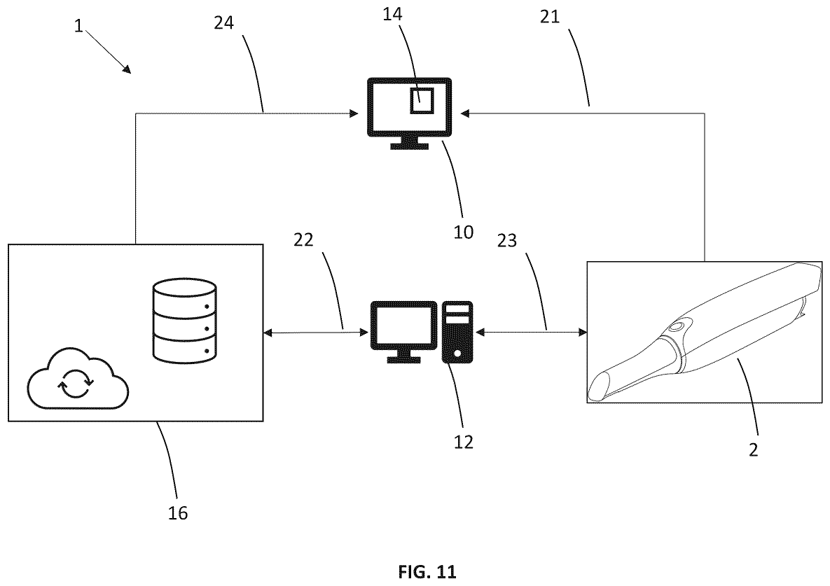

FIG. 11 illustrates exemplary devices that may be used for manufacturing, maintenance, and/or operating an intraoral scanning device 2. FIG. 11 shows an exemplary system 1 and an intraoral scanning device 2. The system 1 may comprise one or more of a manufacturing device 12, a client device 10, and a server device 16 for manufacturing, maintenance, and/or operating the intraoral scanning device 2 in connection with processing intraoral scan data of a patient and providing 2D image data and/or 3D image data.

The manufacturing device 12 may be configured to perform any steps of the method of manufacturing an intraoral scanning device. The manufacturing device 12 may be configured to generate an intraoral scanning device certificate including the intraoral scanning device identifier and at least one of the generated intraoral scanning device keys. The manufacturing device 12 may be configured to transmit the intraoral scanning device certificate to the intraoral scanning device. The manufacturing device 12 may comprise processing elements (such as a processor and a memory)

The intraoral scanning device 2 is configured to perform intraoral scan data and provide 2D image data and/or 3D image data based on processed intraoral scan data. The intraoral scanning device 2 may be configured to communicate with the manufacturing device 12 using e.g., a wireless communication link 23, such as a uni or bidirectional wireless communication link. The wireless communication link may be carried over a short-range communication system, such as WIFI, Bluetooth, or Bluetooth low energy.

The intraoral scanning device 2 may be configured to connect to the client device 10 via a wireless communication link 21, such as a bidirectional wireless communication link. The wireless communication link 21 may be carried over a short-range communication system, such as WIFI, Bluetooth, or Bluetooth low energy. The intraoral scanning device 2 may be configured to connect to the client device 10 over a network. The client device 10 may permit remote updates, such as firmware update, customization update, debug update of the intraoral scanning device where a dispenser connects to the intraoral scanning device via the client device 10 of the user. The client device 10 may comprise a computing device acting as a client, such as a customization device 14 (e.g., a handheld device, a relay, a tablet, a clinic computer, and/or USB dongle plugged in a personal computer). The processing unit/intraoral scanning device is configured to receive a linking request for a session via the interface; and to obtain a session identifier. For example, the interface of the intraoral scanning device 2 is configured to receive the linking request from the client device 10 via communication link 21. For example, the intraoral scanning device 2 receives the linking request from the client device 10 for establishing a communication session. The processing unit of the intraoral scanning device is configured to transmit via the interface a linking response comprising an intraoral scanning device identifier and the session identifier. The processing unit of the intraoral scanning device is configured to receive, via the interface, an authentication message comprising an authentication key identifier and client device data, e.g., from the client device 10 via communication link 21.

The client device 10 may be configured to communicate with the server device 16 via a communication link 24, such as a bidirectional communication link. The communication link 24 may be a wired link and/or wireless communication link. The communication link 24 may comprise a network, such as the Internet.

The client device 10 may be configured to communicate with the server device 16 for maintenance, and update purposes. The server device 16 may comprise a computing device configured to act as a server, i.e., to serve requests from the client device 10 and/or from the intraoral scanning device 2. The server device 16 may be controlled by the intraoral scanning device manufacturer. The server device 16 may be configured to communicate with the manufacturing device 12 via a communication link 22 for manufacturing maintenance, and/or operational purposes. The server device 16 and the manufacturing device 12 may be co-located and/or form one entity for manufacturing maintenance, and/or operational purposes of the intraoral scanning device 2.

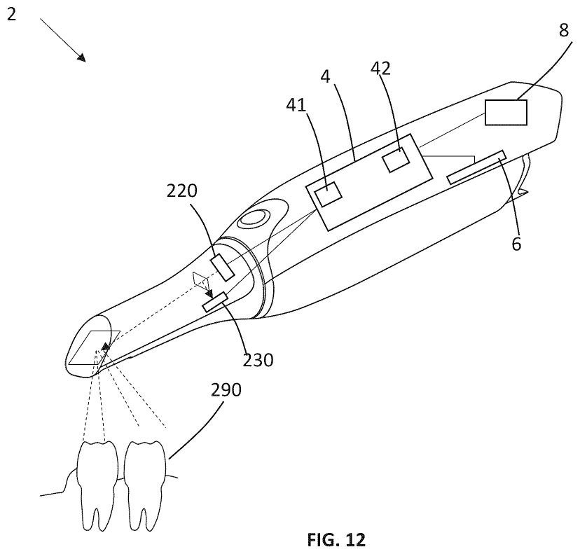

FIG. 12 illustrates an exemplary intraoral scanning device 2. The intraoral scanning device 2 comprises a processing unit 4, a memory unit 6 and a wireless interface 8. The intraoral scanning device 2 comprises a processing unit 4 configured to process intraoral scan data of a patient and provide 2D image data and/or 3D image data. The wireless interface 8 comprises a wireless transceiver, e.g., configured for wireless communication at frequencies in the range from 2.4 to 2.5 GHZ, 2.4 GHz to 5 GHZ, about 2.45 GHz or about 5 GHz. The wireless interface 8 is optionally configured for wireless communication with a manufacturing device 12. The processing unit 4 may be configured to provide 2d image data and/or 3D image data based on intraoral scan data according to data received during manufacture and/or updates, such as customization updates, firmware updates or debug updates. The intraoral scanning device optionally comprises a light projector 220 and an image sensor 230. The light projector includes at least one or more light emitting diodes and/or one or more infrared light sources for emitting light pattern to a three-dimensional dental object 290 of a patient or of a wax model 290 which is a replicate of the patient's dental. The image sensor 230 receives the reflective light from the dental object 290, and the image sensor 230 converts the reflected light into intraoral scan data. The processing unit 4 is then configured to process the intraoral scan data to 2D image data and/or 3D image data. The image data is then forwarded to the wireless interface 8 which transmits the data to an external device.

The processing unit 4 is configured to receive a linking request for a session via the wireless interface 8; and to obtain a session identifier. Hence, the processing unit 4 comprises e.g., an obtain unit 41 configured to obtain a session identifier. Examples of an obtain unit 41 include a random or pseudo-random number generator. The wireless interface is configured to receive the linking request for a session from a client device 10. The processing unit 4 is configured to obtain a session identifier, such as by generating a random or pseudo-random number. The processing unit 4 is configured to store the session identifier in the memory unit 6. The memory unit 6 may be configured to store the session identifier at a memory address of the memory unit 6, and/or in memory cells of the memory unit 6, such as in designated memory cells and/or at designated addresses. The linking request may comprise an authentication key identifier and/or an authentication type identifier, in order to permit the intraoral scanning device 2 to perform authentication at this early stage the linking request and the client device 10 sending the linking request. This may provide a level of access control.

The processing unit 4 is configured to transmit via the wireless interface 8 a linking response comprising an intraoral scanning device identifier and the session identifier. The processing unit 4 may be configured to generate a linking response by including the session identifier and the intraoral scanning device identifier in the linking response. The intraoral scanning device identifier may refer to a unique identifier of the intraoral scanning device 2, such as a serial number, a MAC address, and/or hardware identifier of the intraoral scanning device 2. The wireless interface 8 is configured to transmit the linking response to e.g., the client device 10.

The processing unit 4 is configured to receive, via the wireless interface 8, an authentication message comprising an authentication key identifier and client device data. For example, the wireless interface 8 may be configured to receive the authentication message from the client device 10. For example, the intraoral scanning device 2 receives the authentication message from the client device 10 in order to establish a communication session. The client device data may comprise a client device certificate (encrypted or unencrypted), customization data, intraoral scanning device operating parameters, and/or firmware data. For example, the authentication message may comprise an authentication key identifier in plain text. The authentication key identifier may be indicative of an intraoral scanning device key. The processing unit 4 that processes the authentication key identifier is configured to e.g., verify the authentication key identifier by comparing it to the intraoral scanning device key identifier stored e.g. in the memory unit 6 and determining the authentication key identifier as acceptable if the authentication key identifier is for example equal or higher than the intraoral scanning device key identifier stored.

The processing unit 4 is configured to select an intraoral scanning device key from a plurality of intraoral scanning device keys in the memory unit 6 based on the authentication key identifier. Hence, the processing unit 4 comprises e.g., a select unit 42 configured to select an intraoral scanning device key based on the authentication key identifier. When the authentication key identifier is acceptable by the intraoral scanning device 2 based on an intraoral scanning device key identifier held by the intraoral scanning device 2, the processing unit 4 is configured to select an intraoral scanning device key that the authentication key identifier indicates and to use the selected intraoral scanning device key as keying material used to secure the session. Optionally, the processing unit 4 may be configured to select an intraoral scanning device key from a plurality of intraoral scanning device keys in the memory unit 6 based on the authentication key identifier and an authentication type identifier. The authentication type identifier may be included in the authentication message and received in plaintext by the intraoral scanning device 2, and/or as client device type identifier in the client certificate (encrypted or decrypted). For example, the processing unit 4 may be configured to select an intraoral scanning device key that the authentication key identifier and the authentication type identifier indicate.

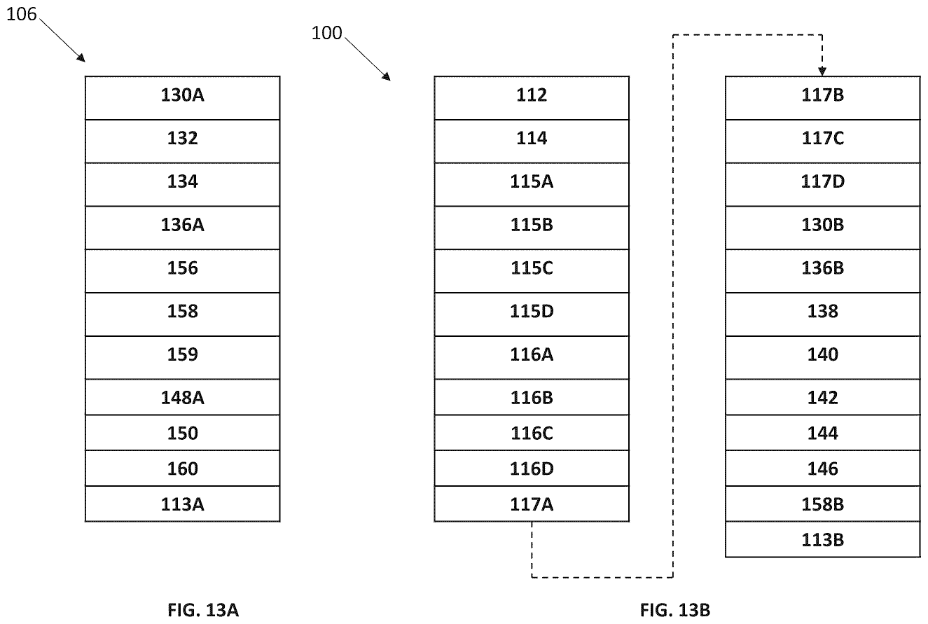

The processing unit 4 is configured to verify the client device data based on the selected intraoral scanning device key; and to terminate the session if verification fails. For example, the processing unit 4 is configured to verify the client device data based on the selected intraoral scanning device key by verifying the integrity of the client device data based on the selected intraoral scanning device key, such as verifying a MAC and/or a digital signature of the client device data. The processing unit 4 is configured to verify the client device data based on the selected intraoral scanning device key by decrypting the client device data using the selected intraoral scanning device key (as keying material to derive a decryption key or as a decryption key), when the client device data is received encrypted, and by verifying the content of the decrypted client device data. The processing unit 4 may be configured to verify the client device data based on the selected intraoral scanning device key by comparing the decrypted client device data with data stored in the memory unit 6. The client device data may comprise a client device certificate (such as an encrypted client device certificate), an authentication key identifier, and/or an authentication type identifier. The client device 10 may be assigned a client device certificate. The client device certificate refers to a certificate generated and assigned to the client device by e.g., a manufacturing device 12. Examples of client device certificates are illustrated in FIG. 13A. The processing unit 4 may be configured to generate a certificate key based on the selected intraoral scanning device key and/or the session identifier. To verify the client device data may comprise to decrypt the encrypted client device certificate with the certificate key to obtain a decrypted version of the encrypted client device certificate.

The processing unit 4 may be configured to verify the client device data by determining if the authentication key identifier matches a client device key identifier of the (decrypted) client device certificate, and verification fails if no match is determined.

In one or more exemplary intraoral scanning devices, the processing unit 4 is configured to verify the client device data by determining if a client device type identifier of the client device certificate is valid and verification fails if the client device type identifier of the client device is not valid. The processing unit 4 is configured to verify the client device data by verifying a digital signature of the client device certificate included in the client device data, and verification fails if the digital signature is not verified.

The processing unit 4 may be configured to verify the client device data by determining if the signing device identifier and/or the client device identifier are valid, e.g., not black-listed.

In one or more exemplary intraoral scanning devices, the processing unit 4 is configured to generate an offline session key based on the common secret and the session identifier, and the processing unit 4 is configured to communicate with the client device using the offline session key.

In one or more exemplary intraoral scanning devices, the authentication message 421 comprises an authentication token identifier, and the processing unit 4 is configured to store the authentication token identifier in the memory unit 6 and to link the authentication token identifier with the common secret. The authentication token identifier may be indicative of enabling a token-based authentication at the intraoral scanning device 2, i.e., when the intraoral scanning device receives an authentication token identifier from an authenticated client device 10, it may enable token-based authentication in future communication with the same client device 10 by storing e.g. an indicator such as a flag in relation with the common secret and the client device. For example, the intraoral scanning device 2 receiving the authentication token identifier may be configured to indicate to the processing unit 4 to enable token-based authentication by storing and/or linking the token identifier with the common secret generated for the same client device 10, such as by storing and/or linking the token identifier with the common secret and the client device identifier of the same client device in e.g., a table.

In the intraoral scanning device 2, the processing unit 4 is configured to generate a session key based on the session identifier and the intraoral scanning device key, and the processing unit 4 is configured to receive and authenticate session data based on the session key.

In one or more exemplary intraoral scanning devices, the processing unit 4 is configured to receive an additional authentication message via the wireless interface 8. The additional authentication message comprises client device data and an authentication device identifier. The processing unit 4 may be configured to obtain a common secret based on the authentication device identifier from the memory unit 6. The processing unit 4 may be configured to generate an additional certificate key from the common secret; and to verify the client device data based on the additional certificate key.

FIG. 13A illustrates an exemplary client device certificate 106. The client device data may comprise a client device certificate 106 and/or an encrypted client device certificate 106A. The client device 10 may be assigned a client device certificate 106. The client device certificate 106 refers to a certificate generated and assigned to the client device 10 by e.g., a manufacturing device 12. The encrypted client device certificate 106A may be generated by the client device 10 using an encryption algorithm and a certificate key.

The client device certificate 106 comprises a certificate type identifier 130A. The certificate type identifier 130A may indicate a type of the certificate amongst a variety of certificate types, such as an intraoral scanning device family certificate type, an intraoral scanning device certificate type, a firmware certificate type, a research and development certificate type, client device certificate type. The certificate type identifier 130A may be used by the intraoral scanning device 2 to identify what type of certificate it receives, stores, and/or retrieves and to act accordingly. The client device certificate 106 may comprise a version identifier 132 which indicates a data format version of the client device certificate 106. The intraoral scanning device 2 may be configured to use the certificate type identifier 130A and/or the version identifier 132 to determine what type of data the certificate comprises, and/or what type of data is comprised in a field of the certificate. For example, the intraoral scanning device 2 determines based on the certificate type identifier 130A and/or version identifier 132 what field of the certificate 106 comprises a digital signature 113A, and/or which public key is needed to verify the digital signature 113A. It may be envisaged that there is a one-to-one mapping between the certificate type identifier 130A and the public-private key pair. It may be envisaged that the intraoral scanning device 2 obtains the corresponding public key, such as retrieves the corresponding public key from the memory unit 6, a remote data storage, and/or receives the corresponding public key from the client device 10 and/or a server device 16. The client device certificate 106 may comprise a signing device identifier 136A. The signing device identifier 136A refers to a unique identifier identifying the device (such as a client device 10, a server device 16, an integrated circuit card, a smart card, and/or a hardware security module thereof) that has signed the client device certificate 106, e.g., during manufacture of the client device. The signing device identifier 136A may for example comprise a medium access control, MAC, address of the signing device, and/or a serial number of the signing device. The signing device identifier 136A allows for example the intraoral scanning device 2 to determine whether the signing device is e.g., black listed or not, and thus to reject certificates signed by a signing device that is black listed. The client device certificate 106 may comprise one or more hardware identifiers, such as a first hardware identifier 148A, and a second hardware identifier 150. The hardware identifiers, 148A, 150 may identify a piece of hardware comprised in the client device 10, such as a radio chip comprised in the client device 10, and/or a digital signal processor of the client device 10. The hardware identifier(s) may be stored in a register of the piece of hardware comprised in the intraoral scanning device during manufacturing of the piece of hardware. The hardware identifier(s) may comprise a serial number, a medium access control, MAC, address, a chip identifier, or any combination thereof.

In one or more exemplary client device certificates, the client device certificate 106 comprises a client device type identifier 156. A client device type identifier 156 may be indicative of a type which the client device belongs to. The client device certificate 106 may comprise a client device identifier 158. The client device certificate may comprise a client device key identifier 159. A client device key identifier 159 may be indicative of the client device key used as keying material for securing a communication with an external party.

In one or more exemplary client device certificates, the client device certificate 106 comprises a Bluetooth address or an IP address (when wireless communication is based on WIFI) 160 of the client device.

The client device certificate 106 comprises a digital signature 113A. The digital signature 113A enables a proof or verification of authenticity of the client device certificate, such as verification of the signer legitimacy. The digital signature 113A is optionally generated by the manufacturing device 12 using a client device customization private key. The intraoral scanning device 2 may be configured to verify the digital signature 113A when receiving the client device certificate comprising the digital signature 113A (i.e., receiving the authentication message comprising the encrypted client device certificate, and obtaining a decrypted version of the client device certificate 106B). The digital signature 113A is verifiable by the intraoral scanning device 2 using a corresponding client device customization public key, which is e.g., stored in the memory unit 6. If the digital signature 113A is not successfully verified using the alleged public key, the intraoral scanning device 2 may disregard the client device certificate 106 (and authentication message) and/or abort normal operation/the session. This may provide the advantage that the intraoral scanning device 2 rejects a client device certificate 106 (and authentication message) that is tampered or received from unauthenticated parties. The communication with the intraoral scanning device 2 may thus be robust against impersonation, modification, and masquerading attacks.

FIG. 13B illustrates an exemplary intraoral scanning device certificate 100. The intraoral scanning device certificate 100 comprises an intraoral scanning device identifier 112, at least one intraoral scanning device key identifier including a first intraoral scanning device key identifier 114 indicative of an intraoral scanning device key and one or a plurality of intraoral scanning device keys. The intraoral scanning device identifier 112 may refer to a unique or a pseudo-unique identifier. The first intraoral scanning device key identifier 114 is indicative of the first intraoral scanning device key(s) of the intraoral scanning device certificate. For example, the first intraoral scanning device key identifier 114 may be indicative of or point to an intraoral scanning device key of a first set 115 of intraoral scanning device keys (115A, 115B, 115C, 115D) of the intraoral scanning device certificate, e.g., the first primary intraoral scanning device key 115A.

The intraoral scanning device certificate 100 optionally comprises at least four sets of intraoral scanning device keys enabling secure and distinct communication with at least four different client devices/client device types.

The intraoral scanning device certificate 100 comprises a first set 115 of intraoral scanning device keys including a first primary intraoral scanning device key 115A. The at least one intraoral scanning device key identifier comprises a first intraoral scanning device key identifier 114 indicative of an intraoral scanning device key of the first set 115 of intraoral scanning device keys 115A, 115B, 115C, 115D. The first set 115 of intraoral scanning device keys comprises for example first primary key 115A, first secondary key 115B, first tertiary key 115C, and first quaternary key 115D dedicated to securing communication to and from a first client device or a first client device type. For example, the first set 115 of intraoral scanning devices key may be a set of intraoral scanning device keys 115A, 115B, 115C, 115D for securing communication of intraoral scanning device data with the first client device.

The plurality of intraoral scanning device keys may comprise a second set 117 of intraoral scanning device keys including a second primary intraoral scanning device key 117A, a second secondary intraoral scanning device key 117B, a second tertiary intraoral scanning device key 117C, and/or a second quaternary intraoral scanning device key 117D. The at least one intraoral scanning device key identifier comprises a second intraoral scanning device key identifier 116 indicative of an intraoral scanning device key of the second set 117 of intraoral scanning device keys 117A, 117B, 117C, 117D. The intraoral scanning device is configured to communicate with one or more client devices, such as a first client device and/or a second client device. For each client device or client device type that the intraoral scanning device is configured to communicate with, the intraoral scanning device certificate optionally comprises a set of intraoral scanning device keys configured to enable secure communication with a specific client device or client device type, and an intraoral scanning device key identifier indicating which intraoral scanning device keys that are part of the intraoral scanning device certificate. The intraoral scanning device certificate may comprise a third set 119 of intraoral scanning device keys including a third primary intraoral scanning device key 119A, a third secondary intraoral scanning device key 119B, a third tertiary intraoral scanning device key 119C, and/or a third quaternary intraoral scanning device key 119D. The at least one intraoral scanning device key identifier comprises a third intraoral scanning device key identifier 118 indicative of an intraoral scanning device key of the third set 119 of intraoral scanning device keys. The intraoral scanning device certificate 100 may comprise a fourth set of intraoral scanning device keys including a fourth primary intraoral scanning device key (not shown). The at least one intraoral scanning device key identifier comprises a fourth intraoral scanning device key identifier indicative of an intraoral scanning device key of the fourth set of intraoral scanning device keys. The intraoral scanning device 2 may be configured to select a set of intraoral scanning device keys based on the client device or the client device type connected to the intraoral scanning device and to select an intraoral scanning device key from the set of intraoral scanning device keys selected based on the intraoral scanning device key identifier associated with the selected set of intraoral scanning devices.

The intraoral scanning device certificate 100 optionally comprises a certificate type identifier 130B. The certificate type identifier 130B indicates that the intraoral scanning device certificate 100 is an intraoral scanning device certificate, e.g., selected amongst a variety of certificate types, such as an intraoral scanning device family certificate type, an intraoral scanning device certificate type, a firmware certificate type, a research and development certificate type, and a client device certificate type. The certificate type identifier 130B may be used to enable the intraoral scanning device 2 to identify what type of certificate it receives, stores, authenticates and/or retrieves. The intraoral scanning device certificate 100 may comprise a version identifier which indicates a data format version of the intraoral scanning device certificate. The intraoral scanning device 2 may use the certificate type identifier 130B and/or the version identifier to determine what type of data the intraoral scanning device certificate 100 comprises, what type of data is comprised in a field of the intraoral scanning device certificate 100. For example, the intraoral scanning device 2 may determine based on the certificate type identifier 130B and/or version identifier what field of the certificate comprises a digital signature 113B, and which public key is needed to verify the digital signature 113B. It may be envisaged that there is a one-to-one mapping between the certificate type identifier 130B, and the public-private key pair used for generating the digital signature 113B. The intraoral scanning device certificate 100 may comprise a length identifier that indicates the length of the intraoral scanning device certificate 100, e.g., in bits, bytes.

The intraoral scanning device certificate 100 optionally comprises a signing device identifier 136B. The signing device identifier 136B refers to a unique identifier identifying the device (such as a manufacturing device 12, e.g., an integrated circuit card, a smart card, a hardware security module comprised in a manufacturing device 12) that has signed the intraoral scanning device certificate 100. The signing device identifier 136B may for example comprise a medium access control, MAC, address of the signing device, a serial number. The signing device identifier 136B allows for example the intraoral scanning device 2 to determine whether the signing device is e.g., black-listed or not, and thus to reject intraoral scanning device certificates 100 signed by a signing device that is black-listed.

The intraoral scanning device certificate 100 optionally comprises one or more hardware identifiers including a first hardware identifier 148B and/or a second hardware identifier (not shown). The first hardware identifier 148B may identify a piece of hardware comprised in the intraoral scanning device 2, such as a processing unit 4, a radio chip comprised in the intraoral scanning device 2, a digital signal processor of the intraoral scanning device 2. The first hardware identifier 148B may also be stored in a register of the piece of hardware comprised in the intraoral scanning device 2 during manufacturing of the piece of hardware. The first hardware identifier 148B may comprise a serial number, a medium access control, MAC, address, a chip identifier, or any combination thereof. The intraoral scanning device certificate 100 may comprise a first hardware identifier 148B, a second hardware identifier and/or a third hardware identifier. For example, the first hardware identifier 148B may provide a first intraoral scanning device specific value present in a register of a hardware module (e.g. the processing unit or the radio chip) of the intraoral scanning device 2 while the second hardware identifier may provide a second intraoral scanning device specific value present in a register of a hardware module of the intraoral scanning device 2, and a third hardware identifier may provide a third hardware module identifier (e.g. a processing unit identifier, a DSP identifier). The intraoral scanning device 2, upon receiving the intraoral scanning device certificate 100 comprising the first hardware identifier 148B, may then verify the intraoral scanning device certificate 100 by comparing its stored hardware identifier and the first hardware identifier 148B comprised in the intraoral scanning device certificate 100 received. This way, the intraoral scanning device 2 may determine if the received intraoral scanning device certificate is intended for the intraoral scanning device 2 and reject the received intraoral scanning device certificate if the stored and received hardware identifiers do not match.

The intraoral scanning device certificate 100 optionally comprises a client device type authorization identifier 144. A client device type may comprise a model, category, or type of client devices, such as a tablet product model, category or type, a USB dongle product model, category or type. The client device type authorization identifier 144 is an identifier of an authorized client device type, such as an identifier of the client device types that the intraoral scanning device 2 may authorize for communication, such as for customizing, maintenance and/or operation. The client device type authorization identifier 144 is for example a bit-field indicating the type of client device the intraoral scanning device 2 should allow for customization.

The intraoral scanning device certificate 100 optionally comprises a token parameter 146. The token parameter 146 indicates whether a token-based authentication is to be enabled or not. For example, if the token parameter 146 is set to 0, token-based authentication of client devices is not to be enabled by the intraoral scanning device 2 and the intraoral scanning device 2 is to use for example a combination of client device type identifier and/or a client device identifier (such as a serial number) to perform an authentication of the client device 10. If for example the token parameter 146 is set to 1, token-based authentication of client devices is to be enabled by the intraoral scanning device 2, i.e., the intraoral scanning device 2 authenticates the client device 10 (such as a based on a token received from the client device 10). The intraoral scanning device 2 may also derive a session specific token based on the received token parameter 146 which is used to e.g., accept the connection to the client device 10 without user intervention.

The intraoral scanning device certificate 100 comprises one or more of a hardware platform identifier 138, a software platform identifier 140, and/or a certificate timestamp 142. The hardware platform identifier 138 may identify a hardware platform, such as an operational intraoral scanning device hardware platform, i.e., a hardware platform on which the intraoral scanning device certificate may be used. The software platform identifier 140 may identify a family of software platforms on which the intraoral scanning device certificate is configured to operate. The certificate timestamp 142 refers to a timestamp of production or manufacture of the intraoral scanning device certificate 100, such as a timestamp of the manufacturing device 12 indicating a time instant when the intraoral scanning device certificate 100 has been generated. The certificate timestamp 142 may be in form of e.g.: hour, min, date, month, year.

The intraoral scanning device certificate comprises a digital signature 113B and/or a MAC. The digital signature 113B enables a proof or verification of authenticity of the intraoral scanning device certificate 100, such as verification of the signer legitimacy (e.g., whether the signer is a legitimate manufacturing device). The digital signature 113B is generated by the manufacturing device 12 using a device family private key during manufacturing of the intraoral scanning device. The intraoral scanning device 2 or the processing unit 4 may then verify the digital signature 113B, e.g., when receiving the intraoral scanning device certificate 100 comprising the digital signature 113B. The digital signature 113B is verifiable by the intraoral scanning device 2 using a corresponding device family public key. If the digital signature 113B is not successfully verified using the alleged public key, the intraoral scanning device may disregard the intraoral scanning device certificate 100 and/or abort normal operation.

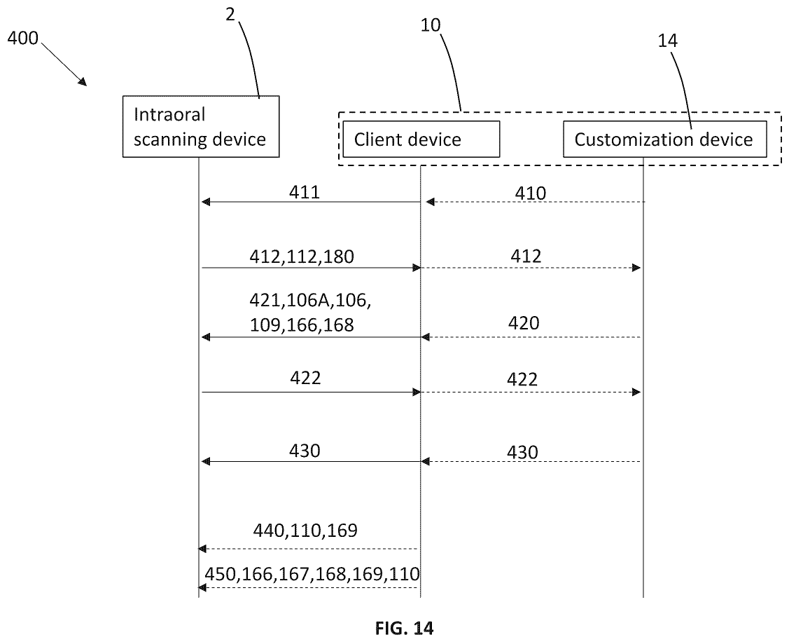

FIG. 14 illustrates an exemplary sequence diagram 400 involving an intraoral scanning device 2, and a client device 10. The client device 10 may comprise a customization device 14. The intraoral scanning device 2 receives via the wireless interface 8 a linking request or message 411 for session from the client device 10. When the client device 10 comprises a customization device 14, the customization device 14 may generate a linking request 410, which is transmitted by the client device 10 as linking request 411. The intraoral scanning device 2 obtains a session identifier 180, such as generates a session identifier 180. The intraoral scanning device 2 generates a linking response 412 comprising an intraoral scanning device identifier 112 and/or a session identifier 180 and transmits the linking response 412 to the client device 10. When the client device 10 comprises a customization device 14, the customization device 10 may receive the linking response 412 via the client device 10. The client device 10 generates an authentication message 421 and transmits the authentication message 421 to the intraoral scanning device 2. The intraoral scanning device 2 receives the authentication message 421 from the client device 10. The authentication message 421 comprises an authentication key identifier 166, optional authentication type identifier 168, and client device data 109. The client device data 109 comprises an encrypted client device certificate 106A or client device certificate 106. Any of client device type identifier 156, client device identifier 158, and a user identifier may be comprised in the encrypted client device certificate 106A. Any of a client device identifier, a client device type identifier 156 and/or a user identifier may be comprised in the authentication message 421 in plain text, or as part of (plain) client device certificate 106. When the client device 10 comprises a customization device 14, the customization device 14 may generate an authentication message 420, which is transmitted by the client device 10 as authentication message 421.

Optionally, the intraoral scanning device 2 is configured to authenticate the authentication message 421 by verifying the content, origin and/or integrity of the authentication message 421. For example, the intraoral scanning device 2 verifies whether the received value of authentication key identifier 166 received is higher or equal to an intraoral scanning device key identifier comprised in the intraoral scanning device certificate 100 (and/or the latest value the intraoral scanning device 2 has stored as intraoral scanning device key identifier in e.g., a flash memory). If the intraoral scanning device 2 determines that the received value of authentication key identifier 166 received is higher or equal to an intraoral scanning device key identifier, the authentication continues, else the session is terminated immediately (with proper error code). This prevents the intraoral scanning device 2 to communicate with an expired/revoked client device.

For example, the intraoral scanning device 2 determines the type of client device based on authentication type identifier. The intraoral scanning device 2 selects an intraoral scanning device key from a plurality of intraoral scanning device keys based on the authentication type identifier 168 and/or the authentication key identifier 166. For example, the intraoral scanning device 2 identifies the intraoral scanning device key corresponding to the authentication key identifier 166 received. The intraoral scanning device 2 uses the identified intraoral scanning device key and the session identifier 180 (e.g., a 16 bytes of random number) sent in linking response 412 to the client device 10 to derive the certificate key, such as to compute a common secret from which the certificate key is derivable.

Using the certificate key, the intraoral scanning device 2 decrypts the encrypted client device certificate 106A. The intraoral scanning device 2 may then verify that the certificate type identifier 130A comprised in the decrypted client device certificate 106B corresponds to the right certificate 106B. The intraoral scanning device 2 may then verify that the authentication type identifier 168 received in plain text match the client device type identifier 156 in the decrypted certificate 106B. The intraoral scanning device 2 may then verify that the authentication key identifier 166 received in plain text match the client device key identifier 159 in the decrypted certificate 106B and may further assess if the authentication key identifier 166 and/or the client device key identifier 159 is indicative of an intraoral scanning device key identifier (such as a first intraoral scanning device key identifier 114) held by the intraoral scanning device 2. The intraoral scanning device 2 may then verify that the version identifier 132 in the decrypted certificate 106B is supported by the intraoral scanning device 2. The intraoral scanning device 2 may then verify that the authentication type identifier 168 received in plain text or the client device type identifier 156 is listed in the client device type authorization identifier 144 of the stored intraoral scanning device certificate 100. The intraoral scanning device 2 may then verify that the authentication type identifier 168 received in plain text or the client device type identifier 156 associated with the first hardware identifier 148A, 150 is not black-listed. The signing device identifier 136A is verified not to be listed on the blacklist. The intraoral scanning device 2 may then verify the digital signature 113A of the client device certificate 106B using the matching public key.

Upon successful authentication of the authentication message 421 and/or verification, the intraoral scanning device 2 may send an authentication response 422 to the client device 10 that may forward it to a customization device 14 when the client device 10 comprises the customization device 14.

The communication channel is now open and secure. The client device 10 or customization device 14 via the client device 10 may send intraoral scanning device data 430 to the intraoral scanning device 2, such as intraoral scanning device data 430 in a session secured by a session key. Intraoral scanning device data 430 comprises e.g., firmware, customization data, and/or intraoral scanning device operating parameters. Customization data may for example be data generated by a customization device 14 used by a dentist. Customization data may comprise setting data, such as power management settings, configuration of a user interface of the intraoral scanning device and/or settings of an optical unit of the intraoral scanning device. Firmware may refer to a computer program provided by the intraoral scanning device manufacturer, and to be installed on the intraoral scanning device 2 to control the intraoral scanning device 2. Firmware is for example to be installed to upgrade the operations and capabilities of the intraoral scanning device 2.

For example, when the client device 10 re-connects to the intraoral scanning device 2, the processing unit 4 may be configured to receive via the interface 8 an additional authentication message 440. The additional authentication message 440 comprises client device data 110 and an authentication device identifier 169. The processing unit 4 may be configured to verify the authentication message 440 and authenticate the client device sending the authentication message 440. The processing unit 4 may be configured to obtain from the memory unit 6, a common secret based on the authentication device identifier 169. The memory unit 6 may have client device identifiers 158 associated with corresponding common secrets stored thereon, for authenticated client devices 10. The processing unit 4 may then be configured to retrieve the corresponding common secret based on the authentication device identifier 169. The common secret has been generated and stored earlier at e.g., an initial round of authentication of a returning client device 10. Thus, once the client device 10 authenticated, the processing unit 4 can just retrieve the corresponding common secret from the memory unit 6. This provides a faster subsequent authentication and avoids having to regenerate the common secret for computing the additional certificate key, and thus saves the corresponding power consumption. The processing unit 4 may be configured to generate an additional certificate key from the common secret and to verify the client device data 110 based on the additional certificate key. For example, the processing unit 4 generates the additional certificate key by computing a hash value based on the common secret and a certificate value. As described above, the processing unit 4 may be configured to verify the client device data 110 based on the additional certificate key by verifying the integrity of the client device data 110, such as verifying a MAC and/or a digital signature of the client device data 110. The processing unit 4 is configured to verify the client device data 110 based on the additional certificate key by decrypting the client device data 110 using the additional certificate key (as a decryption key), when the client device data 110 is received encrypted. The processing unit 4 is configured to verify the client device data 110 by verifying the content of the client device data 110. The processing unit 4 may be configured to verify the client device data by comparing the client device data 110 with data stored in the memory unit 6. The client device data 110 may comprise a client device certificate 106, such as an encrypted client device certificate 106A.

The processing unit 4 may be configured to receive a further authentication message 450 comprising client device data 110 and an authentication token identifier 167. The processing unit 4 is configured to obtain a common secret based on the authentication token identifier 167 from the memory unit 6: to generate a token key from the common secret.

In one or more exemplary intraoral scanning devices, the processing unit 4 is configured to receive a further authentication message 450 comprising client device data 110, an authentication type identifier 168, an authentication key identifier 166 and/or an authentication session token identifier 167. The further authentication message may comprise an authentication device identifier 169. The processing unit 4 may be configured to find in the memory unit 6 the common secret linked to the client device type identifier and/or the client device identifier of the client device 10 that sends the further authentication message 450 based on locating the stored client device type identifier corresponding to the authentication type identifier 168 and/or locating the stored client device identifier corresponding to the authentication device identifier 169. The processing unit 4 may be configured to obtain a common secret based on the authentication type identifier 168: to generate a token key based on the common secret; and to generate a session token identifier based on the token key and the session identifier. The processing unit 4 may have generated in an earlier session with the client device 10 a common secret to e.g., establish a certificate key and may have stored and linked the common secret to the client device type identifier and/or the client device identifier. The processing unit 4 may then be configured to obtain the common secret based on the authentication type identifier 168 and/or the authentication client identifier 169 corresponding to the stored client device type identifier and/or client device identifier. The processing unit 4 may be configured to generate a token key by performing a hash function on the common secret and a token value (such as a pre-defined arbitrary string or a pre-defined arbitrary value). The processing unit 4 may be configured to generate a session token identifier based on the token key and the session identifier by generating a session identifier, and by performing a hash function on the token key and the session identifier. The processing unit 4 may be configured to verify the authentication session token identifier based on the session token identifier. The processing unit 4 may be configured to verify the authentication session token identifier by comparing the authentication session token identifier 167 and the generated session token identifier. For example, if the processing unit determines that the authentication session token identifier 167 matches the generated session token identifier, the verification is successful and the processing unit 4 may proceed with no user physical intervention and continue to verify the client device data 110 provided in the further authentication message 450. The client device data 100 may comprise a client device certificate, which may be verified according to this disclosure.

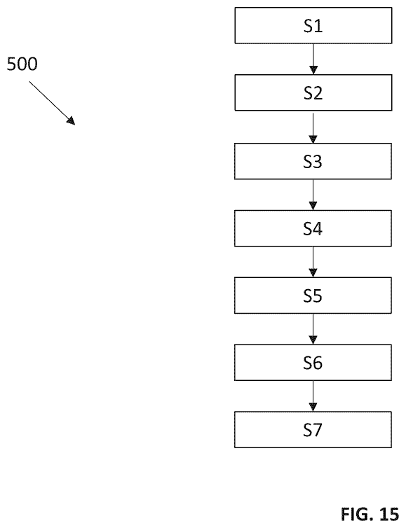

FIG. 15 illustrates a flowchart of an exemplary method 500 of operating an intraoral scanning device 2. The intraoral scanning device 2 comprises a processing unit 4 configured to process intraoral scan data and provide image data, a memory unit 6, and a wireless interface 8. The method 500 comprises receiving S1 a linking request for a session via the wireless interface 8. The linking request may comprise an authentication key identifier 166 and/or an authentication type identifier 168, to allow the intraoral scanning device 2 to perform authentication at this early stage the linking request and the client device sending the linking request. This may provide a level of access control.

The method 500 comprises obtaining S2 a session identifier 180, e.g., with the intraoral scanning device. Obtaining S2 a session identifier 180 may comprise generating a session identifier 180, such as by generating a random or pseudo-random number. For example, the processing unit 4 generates a random or pseudo-random number of a predetermined length, e.g., 16 bits, 32bits, 64bits etc., to be used as a session identifier 180. Obtaining S2 a session identifier 180 may comprise retrieving a session identifier 180 from the memory unit. The method 500 may comprise storing the session identifier 180 in the memory unit 6. For example, storing the session identifier 180 in the memory unit 6 comprises storing the session identifier 180 at a memory address of the memory unit 6, and/or in memory cells of the memory unit 6, such as in designated memory cells and/or at designated addresses.

The method 500 comprises transmitting S3 via the interface 8 a linking response comprising an intraoral scanning device identifier 112 and the session identifier 180. Transmitting S3 the linking response may comprise generating the linking response by including the session identifier 180 and the intraoral scanning device identifier 112 and transmitting the thus generated linking response to e.g., the client device 10.

The method 500 comprises receiving S4, via the interface 8, an authentication message. The authentication message comprises an authentication key identifier 166 and client device data 109. The method 500 may comprise receiving, via wireless the interface 8, an authentication message 421 from a client device 10. For example, the intraoral scanning device 2 receives the authentication message 421 from the client device 10 to establish a communication session. The client device data 109 may comprise a client device certificate, such as unencrypted client device certificate 106 or encrypted client device certificate 106A, customization data, intraoral scanning device operating parameters, and/or firmware data. The authentication key identifier 166 may be an identifier that may be used to verify if the client device 10 has used a client device key acceptable by the intraoral scanning device 2.

The method 500 comprises selecting S5 an intraoral scanning device key from a plurality of intraoral scanning device keys (e.g., within or amongst a first set of intraoral scanning device keys 115, second set of intraoral scanning device keys 116 etc.) in the memory unit 6 based on the authentication key identifier 166. When the authentication key identifier 166 matches the intraoral scanning device key identifier, such as a first intraoral scanning device key identifier 114 held by the intraoral scanning device 2, the processing unit 4 may be configured to use the authentication key identifier 166 as a key identifier indicating which intraoral scanning device key is to be used as keying material in the session. Selecting S5 an intraoral scanning device key from a plurality of intraoral scanning device keys in the memory unit may be based on the authentication key identifier 166 and/or an authentication type identifier 168. The authentication type identifier 168 may be received in plaintext by the intraoral scanning device 2 as part of the authentication message 421, and/or as client device type identifier 156 in the client device certificate 106 (encrypted or decrypted). For example, selecting S5 comprises selecting an intraoral scanning device key that the authentication key identifier 166 and the authentication type identifier 168 indicate.

The method 500 comprises verifying S6 the client device data 109 based on the selected intraoral scanning device key and terminating S7 the session if verification fails. Verifying S6 the client device data 109 based on the selected intraoral scanning device key may comprise verifying the integrity of the client device data 109 based on the selected intraoral scanning device key, such as verifying a MAC and/or a digital signature comprised in the client device data 109. Verifying S6 the client device data 109 based on the selected intraoral scanning device key may comprise decrypting the client device data 109 using the selected intraoral scanning device key (as keying material to derive a decryption key or as a decryption key), when the client device data 109 is received encrypted. Verifying S6 the client device data 109 based on the selected intraoral scanning device key may comprise verifying the client device data 109 by comparing the received client device data 109 with data stored in the memory unit. For example, verification fails and the session is terminated if integrity of the client device data 109 is detected as corrupted by e.g. verifying a MAC or a digital signature, if decryption fails, and/or if comparison of the received client device data 109 (when decrypted if the client device data comprises encrypted data, such as encrypted client device certificate) with data stored in the memory unit, e.g. intraoral scanning device certificate, shows a mismatch or is indicative of corruption.

The authentication message optionally comprises an authentication type identifier 168. An authentication type identifier may be indicative of a client device type identifier 156 and/or a certificate type identifier 130, e.g., included in the client device data (encrypted). Selecting S5 an intraoral scanning device key from a plurality of intraoral scanning device keys may be based on the authentication type identifier 168. Selecting S5 the intraoral scanning device key may be based on the authentication type identifier 168 and/or the authentication key identifier 166 provided in the authentication message 421.

The client device data 109 may comprise a client device certificate 106 or an encrypted client device certificate 106A., an authentication key identifier 166, and/or an authentication type identifier 168. The method 500 may comprise generating S8 a certificate key based on the selected intraoral scanning device key and/or the session identifier 180; and verifying S6 the client device data 109 may comprise decrypting the encrypted client device certificate 106A with the certificate key to obtain a decrypted version 106B of the encrypted client device certificate 106A. Decrypting the encrypted client device certificate 106A with the certificate key may comprise decrypting the encrypted client device certificate 106A using a certificate key, a common secret and/or an intraoral scanning device key, such as generating a certificate key based on a common secret, and processing the encrypted client device certificate 106A using a decryption function and a certificate key. The certificate key may be based on a common secret and/or a certificate value. Generating a certificate key may comprise obtaining or generating the common secret based on the selected intraoral scanning device key. For example, generating the common secret based on the intraoral scanning device key comprises retrieving the intraoral scanning device certificate 100 from the memory unit 6, the intraoral scanning device certificate 100 comprising the selected intraoral scanning device key, and/or retrieving the selected intraoral scanning device key from the memory unit 6. The method may comprise generating the common secret based on a session identifier 180 and/or the selected intraoral scanning device key (e.g., the first primary intraoral scanning device key 115A). For example, the common secret CS is generated based on a selected intraoral scanning device key and a session identifier 180, e.g., as follows:

$$CS = hash ( IOS\_KEY , S\_ID ) ,$$

where hash is a hash function, IOS_KEY is the selected intraoral scanning device key and S_ID is a session identifier 180. The session identifier 180 may comprise a random or pseudo random number of a defined length. The common secret may be used as a certificate key in one or more exemplary methods. The method 500 may comprise storing the common secret in the memory unit 6, so as to e.g., retrieve the common secret from the memory unit 6 when needed.

Generating a certificate key may comprise performing a hash function on the common secret and/or a certificate value. Generating the certificate key may be performed e.g., as follows:

$$C\_KEY = hash ( CS , C\_VAL ) ,$$

where hash is a hash function, CS is the common secret and C_VAL is a certificate value. The certificate value may be a predefined value or string, such as the string “certificate”.

In one or more exemplary methods, generating a certificate key comprises performing a hash function on the intraoral scanning device key (e.g., the first primary intraoral scanning device key 115A) and the session identifier 180. Stated differently, the common secret may be used as a certificate key if the client device has also used the common secret as certificate key to encrypt the client device certificate 106.

Verifying S6 the client device data 109 may comprise decrypting the encrypted client device certificate 106A using the certificate key generated by the intraoral scanning device 2 and obtaining the decrypted version 106B of the encrypted client device certificate 106A.

In one or more exemplary methods, verifying S6 the client device data 109 may comprise verifying a content of the decrypted version 106B of the encrypted client device certificate 106A, e.g., based on an intraoral scanning device certificate or at least parts thereof.

For example, verifying S6 (with the intraoral scanning device) the client device data 109 comprises determining if the authentication key identifier 166 matches a client device key identifier 159 of the client device certificate 106, and verification fails if no match is determined.

In one or more exemplary methods, verifying S6 the client device data 109 comprises determining if a client device type identifier 156 of the client device certificate 106 or 106B is valid and verification fails if the client device type identifier 156 of the client device certificate 106 or 106B is not valid. For example, an authentication type identifier 168 is sent in plain text in the authentication message 421, the authentication type identifier 168 sent in plain text is valid if the authentication type identifier 168 matches a corresponding client device type identifier 156 comprised in the decrypted version 106B of the client device certificate 106. For example, determining if a client device type identifier 156 of the client device certificate 106 is valid comprises determining if the client device type identifier 156 of the client device certificate 106 is comprised in a list of authorized client devices stored in the memory unit 6 and/or retrieved from remote data storage.

In one or more exemplary methods, verifying S6 the client device data 109 comprises verifying a digital signature 113A of the client device certificate 106, 106B, and verification fails if the digital signature is not verified. For example, the client device data 109 comprises a digital signature 113A included in or appended to the client device data 109 to protect integrity of the client device data 109. Verifying a digital signature 113A comprises e.g., computing a comparison result based on the digital signature 113A and a corresponding client device public key and comparing the comparison result to the received client device data 109. Verifying a digital signature 113A may comprise retrieving the corresponding client device public key from the memory unit 6 and/or from remote data storage. The digital signature 113A is verified as valid, or the verification is successful when the digital signature 113A raised to the power of the corresponding client device public key is identical to the received client device data 109.

In one or more exemplary methods, the client device certificate 106 comprises a signing device identifier 136A and/or a client device identifier 158 and verifying S6 the client device data 109 comprises determining if the signing device identifier 136A and/or the client device identifier 158 of the client device certificate 106 or 106B is valid and wherein verification fails if the client device identifier 158 and/or the signing device identifier 136A is not valid.

In one or more exemplary methods, the method 500 comprises receiving an additional authentication message 440 comprising client device data 110 and an authentication device identifier 169. The method may further comprise obtaining, from the memory unit 6, a common secret based on the authentication device identifier 169, generating an additional certificate key from the common secret, and verifying the client device data 110 based on the additional certificate key.

In one or more exemplary methods, the method 500 comprises generating an offline session key based on the common secret and the session identifier 180 and communicating with the client device 10 using the offline session key.

In one or more exemplary methods, the method 500, the authentication message 421 comprises an authentication token identifier, and the method 500 comprises storing the authentication token identifier in the memory unit 6 and linking the authentication token identifier with the common secret.

In one or more exemplary methods, the method 500 comprises receiving a further authentication message 450 comprising client device data 110, an authentication type identifier 168, an authentication key identifier 166 and/or an authentication session token identifier 167. The method 500 may comprise obtaining a common secret based on the authentication type identifier 168, generating a token key based on the common secret; and generating a session token identifier based on the token key and the session identifier. The method 500 may comprise verifying the authentication session token identifier 167 based on the session token identifier.

In one or more exemplary methods, the method 500 comprises generating a session key based on the session identifier 180 and the intraoral scanning device key, receiving and authenticating session data (e.g., intraoral scanning device data 430) based on the session key.

### Dental System, Intraoral Scanning Devices, and Method of Securing Communication for a User Application:

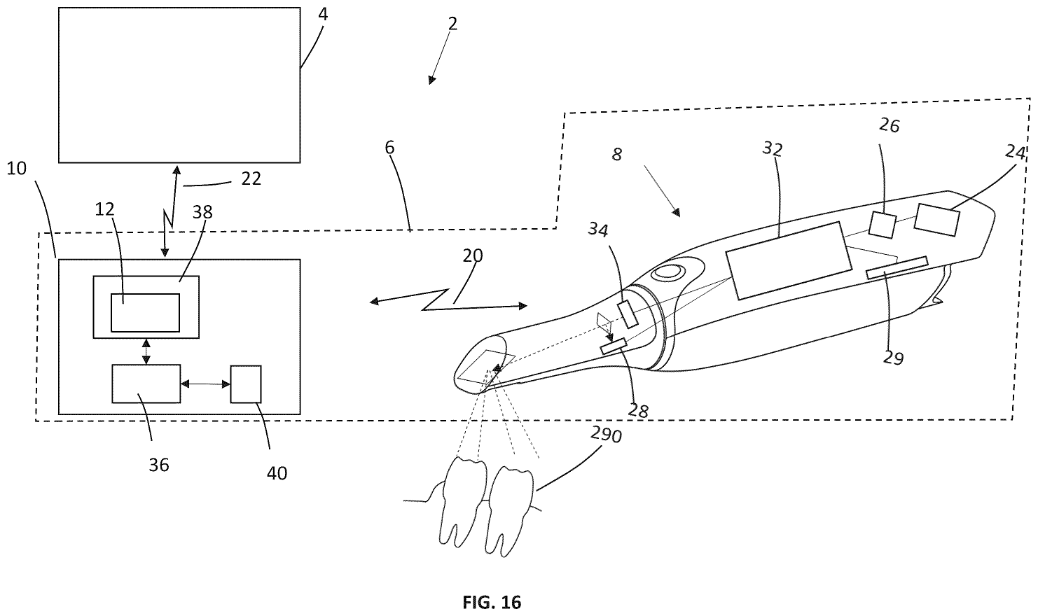

FIG. 16 shows an exemplary dental system 2. The dental system 2 comprises a server device 4 and an intraoral scanning device system 6 comprising an intraoral scanning device 8 and an external device 10. The external device 10 is a tablet computer configured to wirelessly communicate with the intraoral scanning device 8. A user application 12 is installed on the external device 10. The user application may be for controlling the intraoral scanning device 8. In one or more exemplary user applications, the user application 12 is configured to transfer firmware and/or customization data, such as intraoral scanning device settings, to the intraoral scanning device.

The server device 4 and/or the user application 12 may be configured to perform any acts of the method disclosed herein. The intraoral scanning device 8 may be configured to acquire intraoral scan data from a three-dimensional dental object 290 and process the intraoral scan data of a patient and provide 2D image data and/or 3D image data. The intraoral scanning device 8 is configured to configured to communicate with the external device 10/user application 12, e.g., using a wireless communication link 20. The first communication link 20 may be a single hop communication link or a multi-hop communication link. The first communication link 20 may be carried over a short-range communication system, such as Bluetooth, Bluetooth low energy, or WIFI.

The external device 10/user application 12 is configured to connect to the server device 4 over a network, such as the Internet and/or a mobile phone network, via a second communication link 22. The server device 4 may be controlled by the intraoral scanning device manufacturer. The intraoral scanning device 8 comprises an antenna 24 and a radio transceiver 26 coupled to the antenna 4 for receiving/transmitting wireless communication including first communication link 20. The intraoral scanning device 8 comprises a projector 34 configured to emit light onto a dental object 290, and the device 8 comprises a camera 28 configured to receive reflected light of the dental object. The intraoral scanning device 8 comprises a memory unit 29 connected to the processor 32, wherein intraoral scanning device settings are stored in the memory unit.

The intraoral scanning device 2 comprises a processor 32 connected to the transceiver 26, the projector 34 and the camera 28 for receiving and processing intraoral scan data. The processor 32 is configured to process the intraoral scan data and provide 2D image data and/or 3D image data.

The external device 10 comprises a processing unit 36, a memory unit 38, and a wireless interface 40. The user application 12 is installed in the memory unit 38 of the external device 10 and is configured to secure communication for the user application, wherein to secure communication for the user application comprises to obtain challenge data from the server device 4: transmit a challenge request comprising the challenge data to the intraoral scanning device 8: receive a challenge response comprising response data from the intraoral scanning device 8; and transmit the response data to the server device 4.

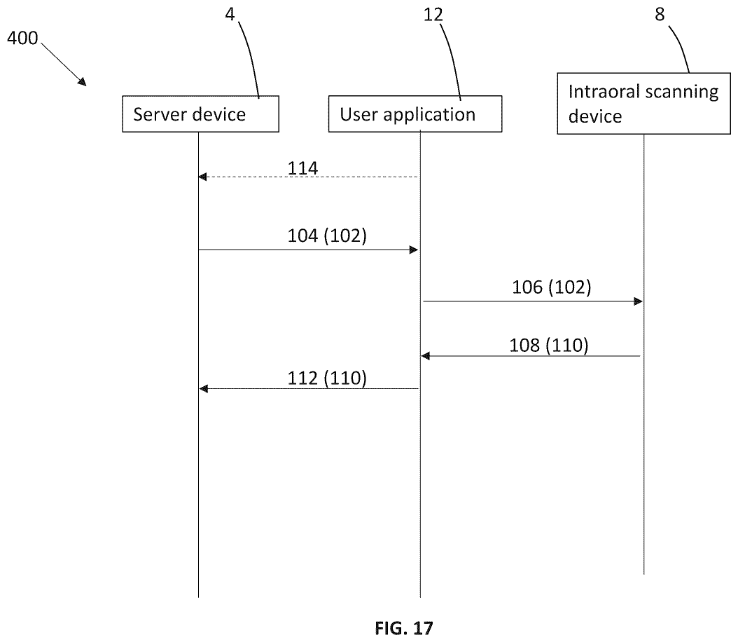

FIG. 17 shows an exemplary sequence diagram 100 between the entities 4, 8, 12 of the dental system 2 illustrating an exemplary method of securing communication for a user application on an external device of a dental system comprising an intraoral scanning device. Securing communication for the user application comprises obtaining challenge data in the server device 4. The method comprises transmitting the challenge data 102 in a challenge message 104 from the server device 4 to the user application 12. The user application 12 receives the challenge data and transmits a challenge request 106 comprising the challenge data 102 from the user application 12 to the intraoral scanning device 8. The intraoral scanning device 8 generates response data based on the challenge data and optionally an intraoral scanning device identifier of the intraoral scanning device and transmits a challenge response 108 to the user application 12, the user application receiving the challenge response 108 comprising response data 110 from the intraoral scanning device 8. The user application forwards the response data 110 in a response message 112 to the server device 4, and the server device 4 verifies the response data 110 based on the challenge data and approves the user application 12 in the server device 4 if verifying the response data 110 is successful.

Optionally, the method comprises transmitting a request 114 for challenge data from the user application 12 to the server device, e.g., if a first approval criterion is fulfilled. In the illustrated dental system, the first approval criterion is fulfilled if the user application has started for the first time, or the user application has been updated. Receipt of the request for challenge data in the server device 4, i.e., a first approval criterion fulfilled in server device, triggers securing communication for the user application. The server device 4 is configured to determine if an approval criterion is fulfilled and the server device 4 is configured to initiate securing communication for the user application if the approval criterion is fulfilled. The approval criterion in the server device comprises a first approval criterion and optionally a second approval criterion. The second approval criterion is fulfilled if the user application has not been approved for a certain period of time, e.g., 14 days. Thus, the second approval criterion may be based on an approval timestamp indicative of time of last approval of the user application. The approval criterion is fulfilled if the first approval criterion or the second approval criterion is fulfilled.

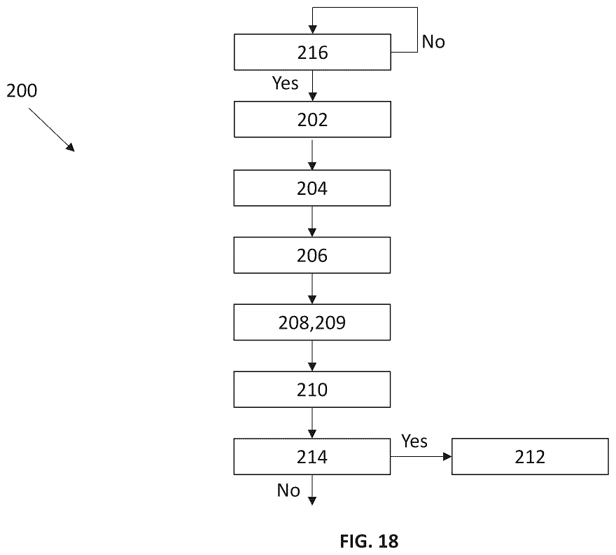

FIG. 18 shows a flow diagram of an exemplary method of securing communication for a user application on an external device of a dental system comprising an intraoral scanning device. In the method 200, securing communication for the user application comprises obtaining 202 challenge data in a server device: transmitting 204 the challenge data from the server device to the user application: transmitting 206 a challenge request comprising the challenge data from the user application to the intraoral scanning device: receiving 208 a challenge response comprising response data from the intraoral scanning device; and forwarding 209 the response data from the user application to the server device. The method 200 comprises verifying 210 the response data in the server device based on the challenge data; and approving 212 the user application in the server device if verifying the response data is successful 214. Optionally, the method comprises determining 216 if an approval criterion is fulfilled in the server device and proceed with obtaining 202 challenge data if the approval criterion is met. If so, the method initiates or proceeds to securing communication for the user application.

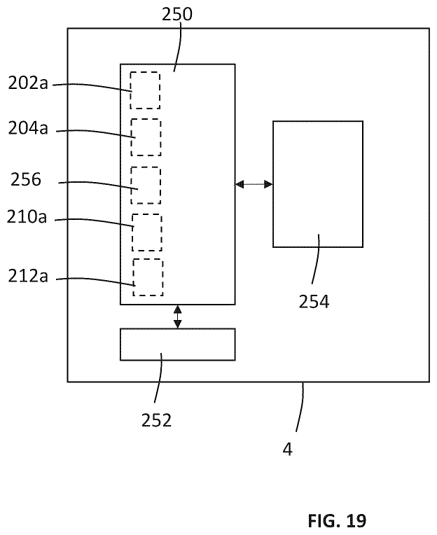

FIG. 19 shows an exemplary server device for securing communication for a user application on an external device of a dental system 2 comprising an intraoral scanning device 8. The server device 4 comprises a processing unit 250, a memory unit 252, e.g., comprising a database, and an interface 254. The server device 4 is configured to approve the user application, wherein to approve the user application comprises to obtain challenge data, e.g., with obtain module 202a. To obtain challenge data comprises to generate challenge data, e.g., based on a default challenge value and/or a timestamp. The challenge data has a length of 16 bytes. The server device is configured to transmit the challenge data via the interface 254 to a user application of an external device in a challenge message, e.g., with transmit module 204a, and receive a response message comprising response data from the user application via the interface 254, e.g. by receive module 256. The server device is configured to verify the response data, e.g., a response value of the response data, based on the challenge data and/or an intraoral scanning device identifier, e.g. by verification module 210a. The server device may comprise a hardware security module, e.g., as part of verification module 210a, configured to verify the response data/response value. If the response data are verified, the server device is configured to approve the user application, e.g., with approval module 212a. To verify the response data optionally comprises calculating the challenge data and verify the response data based on the calculated challenge data. Calculating challenge data as part of the response data verification eliminates the need for memory in the server device and storing of challenge data. To verify the response data comprises to verify a response value of the response data e.g., based on the challenge data and/or an intraoral scanning device identifier of the response data. To verify the response data in the server device may comprise to verify a checksum value of the response data.

The server device 4 is optionally configured to receive a request for challenge data from the user application via the interface 254, and to initiate securing communication for the user application upon receipt of the request for challenge data from the user application. Further, the server device 4 is optionally configured to determine if a second approval criterion based on a last approval timestamp is fulfilled; and to initiate approval of the user application if the second approval criterion is fulfilled, e.g., if the user application has not been approved for a certain period of time, e.g. 14 days.

The server device 4 may be arranged to execute at least parts of methods of securing communication for a user application on an external device of a dental system as disclosed herein. The server device or the processing unit 250 may further comprise a number of optional functional modules, such as any of an obtain module 202a configured to perform step 202, a transmit module 204a configured to perform step 204, a receive module 256 configured to receive a response message, a verification module 210a configured to perform step 210, and an approval module 212a configured to perform step 212. In general terms, each functional module may be implemented in hardware or in software.

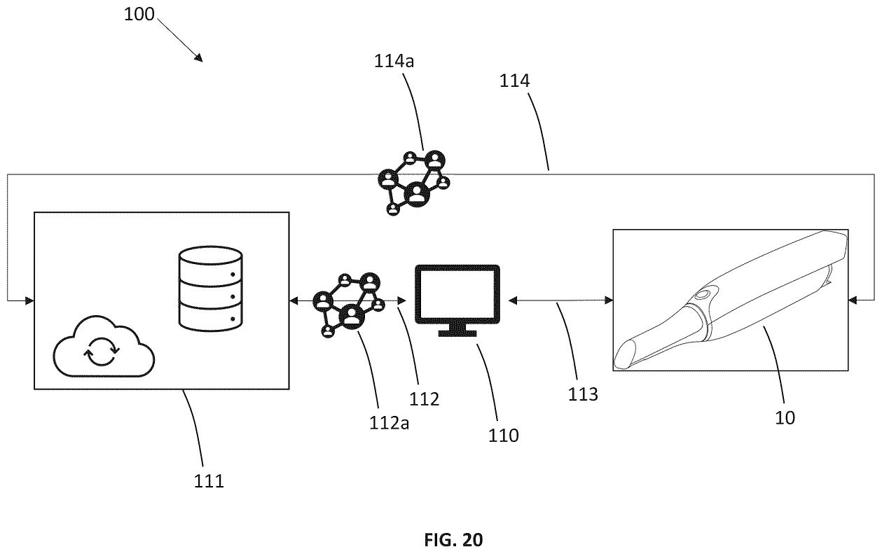

FIG. 20 illustrates an exemplary architecture 100 according to this disclosure. The architecture 100 comprises an intraoral scanning device 10, a client device 110, and a session key apparatus 111. The client device 110 may comprise a computing device acting as a client, a customization device, a handheld device, a relay, a tablet, a personal computer, a mobile phone, and/or USB dongle plugged into a personal computer. The session key apparatus 111 may comprise a computing device configured to act as a server, i.e., to serve requests from the client device 110 and/or from the intraoral scanning device 10. The server device 111 may be controlled by the intraoral scanning device manufacturer.

The intraoral scanning device 101 may be connected to the client device 110 via a communication link 113, such as a wireless communication link or a bidirectional wireless communication link. The wireless communication link may be carried over a short-range communication system, such as Bluetooth, Bluetooth low energy, IEEE 802.11, Zigbee, WIFI. The intraoral scanning device 101 may be connected to the client device 110 over a network.

The intraoral scanning device 101 may be connected to the session key apparatus 111 via a wireless communication link 114 or a bidirectional wireless communication link 114 over a network 114a, such as a bidirectional wireless communication link and/or wireless communication link over a network.

The client device 110 may be connected to the session key apparatus 111 via a communication link 112 over a network 112a, such as a bidirectional wireless communication link and/or wireless communication link over a network. In an embodiment, the network 112a may be the Internet.

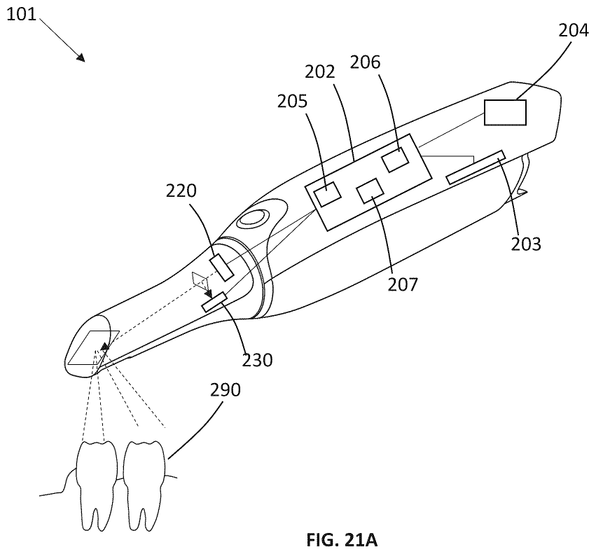

FIG. 21A illustrates an exemplary intraoral scanning device 10. The exemplary intraoral scanning device 101 comprises a processing unit 202 configured to process intraoral scan data of a patient 290 and provide 2D image data and/or 3D image data. The exemplary intraoral scanning device 101 comprises a memory and a wireless interface 204. The memory is in FIG. 21A/21B illustrated in the form of a memory unit 203 external to the processing unit 202. The memory may in other exemplary intraoral scanning devices be at least partly embedded in the processing unit 202 and/or in the memory unit 203.

The processing unit 202 is configured to receive a mode request via the wireless interface 204. Hence, the processing unit 202 comprises a receive/send unit 205 configured to send and/or receive via the wireless interface 204. The receive/send unit 205 is configured to send and receive via the wireless interface 204 to/from an external device, such as a server device, a client device, a customization device, an accessory, a relay device, a smart phone. The processing unit 202 is configured to authenticate the mode request. Hence, the processing unit 202 may comprise an authenticator 206 configured to authenticate the mode request. The processing unit 202 is configured to place the intraoral scanning device into the requested mode, such as a service mode, a customization mode, an upgrade mode, or debug mode, if authentication of the mode request succeeds. Hence the processing unit 202 comprises a mode controller 207 configured to place the intraoral scanning device 101 into the requested mode, e.g., based on an output from the authenticator 206. In the intraoral scanning device in FIG. 21A, the processing unit 202 is configured to place the intraoral scanning device into a default mode if authentication of the mode request fails, the default mode comprising booting the intraoral scanning device and operating the intraoral scanning device according to operating parameters set during booting. In an embodiment, the operating parameters set during booting may be stored in a non-volatile part of the memory unit 203. In an embodiment, the operating parameters set during booting may comprise a default setting enabling the intraoral scanning device to function according to a default setting programmed during production of the intraoral scanning device.

The intraoral scanning device comprises a light projector 220 and an image sensor 230. The light projector includes at least one or more light emitting diodes and/or one or more infrared light source for emitting light pattern to a three-dimensional dental object 290 of a patient or of a wax model 290 which is a replicate of the patient's dental. The image sensor 230 receives the reflective light from the dental object 290, and the image sensor 230 converts the reflected light into intraoral scan data. The processing unit 202 is then configured to process the intraoral scan data to 2D image data and/or 3D image data. The image data is then forwarded to the wireless interface 204 which transmits the data to an external device.

FIG. 21B schematically illustrates an exemplary client device 110. The exemplary client device 110 comprises a processing unit 202 configured communicate with an intraoral scanning device 101. The exemplary client device 110 comprises a memory unit 203 and a wireless interface 204 respectively connected to the processing unit 202. The processing unit 202 is configured to send a session request for a session to the intraoral scanning device via the wireless interface. The session request may comprise the intraoral scanning device identifier or an identifier derived therefrom. The processing unit 202 is configured to receive a session response via the wireless interface, e.g., from an intraoral scanning device 101 and/or a session key apparatus 111. Hence, the processing unit 202 comprises e.g., a receive/send unit 205 configured to send and/or receive via the wireless interface 204. The processing unit 202 is configured to obtain a session key based on e.g., the session response, such as to extract the session key from the session response. Hence, the processing unit 202 comprises an obtainer 206. The processing unit 202 may retrieve the session key from a key depository, e.g., stored in the memory unit 203.

To obtain a session key may comprise validating the intraoral scanning device identifier based on a client device key and derive the session key based on the intraoral scanning device identifier.

Alternatively, to obtain a session key may comprise to establish a connection to a session key apparatus 111 via the wireless interface 204, i.e., the processing unit 202 may be configured to establish a connection to a session key apparatus via the wireless interface. The processing unit 202 may send a session key request, e.g., comprising an intraoral scanning device identifier, to the session key apparatus via the wireless interface 204. The processing unit 202 may receive a session key response from the session key apparatus 111 via the wireless interface 204, the session key response optionally comprising an intraoral scanning device key. The processing unit 202 may determine the session key based on the session key response, e.g., by decrypting the encrypted session key with the intraoral scanning device key from the session key apparatus.

The session response may comprise an intraoral scanning device identifier. The intraoral scanning device identifier may comprise a hardware number of the intraoral scanning device and/or a serial number of the intraoral scanning device. The client device 110 may retrieve the session key from the session key apparatus 111 by providing the intraoral scanning identifier to the session key apparatus 111 and requesting the session key from the session key apparatus 111 and/or requesting the session key apparatus 111 to decrypt the session response and/or the session key.

In one or more exemplary client devices, to obtain the session may comprise to establish a connection to a session key apparatus via the wireless interface, to send a session key request to the session key apparatus via the wireless interface, to receive a session key response from the session key apparatus via the wireless interface, and to determine the session key based on the session key response. The connection establishment may be a secure connection establishment e.g., using a secure protocol to a server acting as session key apparatus.

The processing unit 202 is configured to determine intraoral scanning device data. Hence the processing comprises e.g., a determiner. The intraoral scanning device data comprises e.g., firmware, customization data, and/or intraoral scanning device operating parameters. Thus, the client device 110 may be a customization computer or a dentist computer. Customization data may for example be data generated by a customization computer used by a dentist. Customization data may comprise setting data of the intraoral scanning device, such as power management settings, configuration settings, configuration of a user interface of the intraoral scanning device and/or settings of an optical unit of the intraoral scanning device. Intraoral scanning device operation parameters may comprise mode and/or program control parameters. Firmware may refer to a computer program provided by the intraoral scanning device manufacturer, and to be installed on the intraoral scanning device to control the intraoral scanning device. Firmware is for example to be installed to upgrade the operations and capabilities of the intraoral scanning device.

The client device may be in the form of a desktop computer, a laptop, or a tablet that comprises software configured to provide the functionality of a customization computer.

The session response may comprise an encrypted session key. The processing unit 202 may be configured to determine the session key by retrieving the session key from the session key response

In one or more exemplary client devices, to determine the session key comprises retrieving an intraoral scanning device key from the session key response and decrypting the encrypted session key based on the intraoral scanning device key. Alternatively, to determine the session key may comprise decrypting the encrypted session key with a global key. A global is e.g., a key common to a group of client devices. The processing unit 202 is configured to e.g., retrieve an intraoral scanning device key from the session key response and decrypt the encrypted session key based on the intraoral scanning device key. The processing unit may comprise a decrypt/encrypt unit. The intraoral scanning device key may be e.g., a symmetric key or a public key of a private-public key pair. The intraoral scanning device key may comprise an AES-128 bits key as a symmetric key. The use of a symmetric as an intraoral scanning device key provides the advantage of being able to use hardware accelerators. The intraoral scanning device key may comprise a public key of a private-public key pair, such as a public key of a private-public key pair of an authorized discloser of the session key, such as of the client device 110 or the session key apparatus 111.

In one or more exemplary client devices, to determine the session key comprises decrypting the encrypted session key with a global key. The global key may be e.g., a symmetric key or a public key of a private-public key pair. The session key may be compliant with or encrypted with an encryption standard such as Advanced Encryption Standard, AES, RSA crypto-system, Triple Data Encryption Algorithm.

The processing unit 202 is configured to generate session data e.g., based on the session key and the intraoral scanning device data. Hence the processing comprises e.g., a generator 208. The processing unit 202 may generate a message authentication code based on the session key and the intraoral scanning device data. A message authentication code, MAC, is generated by a client device 110 based on the session data and the session key. Upon reception of the session data comprising the MAC, the intraoral scanning device which holds the stored session key is able to re-compute the MAC based on the received session data and a MAC generation function and compare the recomputed MAC with the received MAC. If the recomputed MAC does not match the received MAC, then the intraoral scanning device concludes that session data is corrupted. The processing unit 202 to generate session data may be configured to encrypt the intraoral scanning device data with the session, using e.g., an encryption scheme. The encryption scheme may comprise a symmetric encryption scheme and/or an asymmetric encryption scheme. The session key may comprise a symmetric key. The symmetric key may be uniquely generated for each session by the intraoral scanning device and/or session key apparatus. The symmetric key may comprise an AES-128 bits key. The use of a symmetric key as a session key may reduce a processing power requirement and allow the intraoral scanning device 101 to decrypt the session data with light encryption algorithms.

The processing unit 202 may be configured to digitally sign the intraoral scanning device data, such as to digitally sign the intraoral scanning device data using a private key of the client device, and/or of a group of client devices. The processing unit 202 generates a digital signature using a signature generation function and a private key of a client device 110 and append it to the session data. The intraoral scanning device 101 may then verify the digital signature when receiving the session data. If the digital signature is not successfully verified using the alleged public key of a client device 110, the intraoral scanning device 101 may disregard the session data and terminates the session. This may provide the advantage that the client device 110 supports the intraoral scanning device 101 in rejecting session data tampered or received from unauthenticated parties and the communication with the intraoral scanning device may thus be robust against impersonation and masquerading attacks.

The processing unit 202 is configured to send the session data to the intraoral scanning device via the wireless interface, e.g., using the receive/send unit 205. The session data may comprise intraoral scanning device data encrypted with the session key. To encrypt session data with the session key, the client device 110 may utilize any of the above encryption standards.

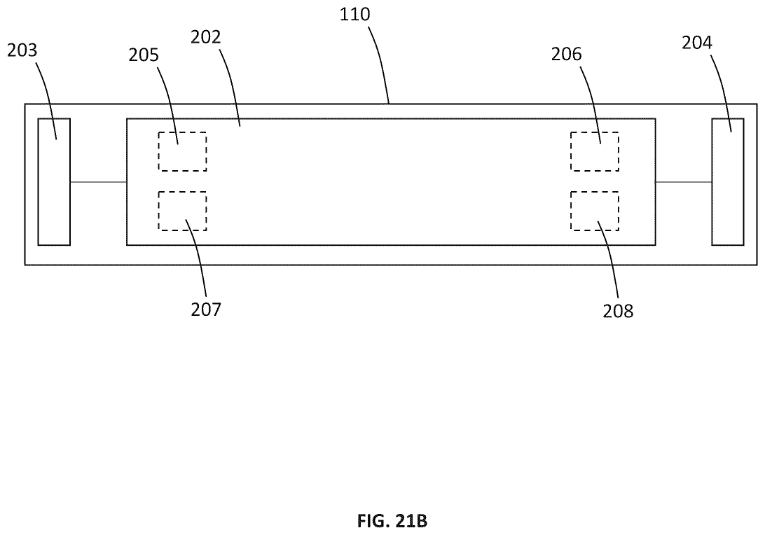

FIG. 22 shows an exemplary sequence diagram 300 involving a session key apparatus 111. A client device 110 sends to e.g., the intraoral scanning device 101 a session request 301 for a session via the wireless interface 204. The client device 110 receives from e.g., intraoral scanning device 101 a session response 302. The session response 302 may comprise an encrypted session key. The client device 110 obtains the session key. For example, to obtain the session key comprises validating the intraoral scanning device identifier based on a client device key and derive the session key based on the intraoral scanning device identifier. Alternatively, to obtain session key, the client device 110 may send a session key request 304 to the session key apparatus 111, whereupon the session key apparatus decrypts the encrypted session key comprised in the session response 302. Based on the session key request 304, the session key apparatus 111 may send the decrypted session key or an intraoral scanning device key enabling the client device to decrypt the encrypted session key in a session key response 305 to the client device 110. The session key response 305 from the session key apparatus 111 may comprise the decrypted session key. The communication link 112 between the client device 110 and the session key apparatus 111 is secure. For example, the session key apparatus 111 may be physically attached as a USB dongle to the client device 111. Alternatively, the session key apparatus 111 may be connected to the client device via a network. The communication link 112 between the client device 110 and the session key apparatus 111 may be secure, i.e., authenticated, encrypted and/or integrity protected using a security protocol (e.g., Transport Layer Security protocol). The client device 110 determines intraoral scanning device data and generates session data 303 based on the session key and the intraoral scanning device data. For example, the client device 110 encrypts the intraoral scanning device data with the session key. The session data 303 may be integrity-protected by the client device 110. The client device 110 sends the session data 303 in the session via the wireless interface 204.

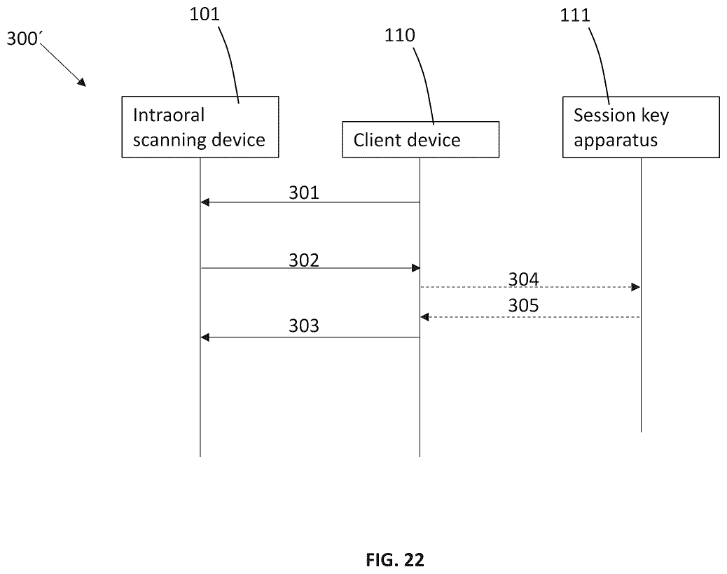

FIG. 23 shows an exemplary flowchart illustrating a method, performed in a client device 110, for intraoral scanning device communication. The client device 110 comprises a processing unit 202, a memory unit 203 and a wireless interface 204. The method comprises sending A1 a session request for a session to the intraoral scanning device 101 via the wireless interface 204, e.g., to an intraoral scanning device 101 and/or a session key apparatus 111. The method comprises receiving A2 a session response via the wireless interface 204, from an intraoral scanning device 101 and/or a session key apparatus 111.

The method comprises obtaining A3 a session key based on the session response, such as to extract the session key from the session response. Obtaining A3 a session key may comprise establishing A31 a connection to a session key apparatus 111, sending A32 a session key request to the session key apparatus 111, receiving A33 a session key response from the session key apparatus 111, and determining A34 the session key based on the session key response. The client device 110 may retrieve the session key from the session key apparatus 111 by providing the intraoral scanning device identifier to the session key apparatus 111 and requesting the session key from the session key apparatus 111 and/or requesting the session key apparatus 111 to decrypt the session response and/or the session key

In one or more methods, receiving A2 a session response may comprise receiving A21 an encrypted session key. Determining A33 the session key may comprise retrieving A33a the session key from the session key response. Determining A33 the session key may comprise retrieving A33b an intraoral scanning device key from the session key response and decrypting A33c the encrypted session key based on the intraoral scanning device key.

In one or more methods, receiving A2 a session response may comprise receiving A21 an encrypted session key. Obtaining A3 a session key based on the session response may comprise decrypting A35 the encrypted session key with a global key. The global key may be stored in the client device 110, such as in the memory 203.

The method comprises determining A4 intraoral scanning device data. The method comprises generating A5 session data based on the session key and the intraoral scanning device data. The method comprises sending A6 the session data to the intraoral scanning device via the wireless interface.

### Method of Controlling Access to Intraoral Scanning Device Services:

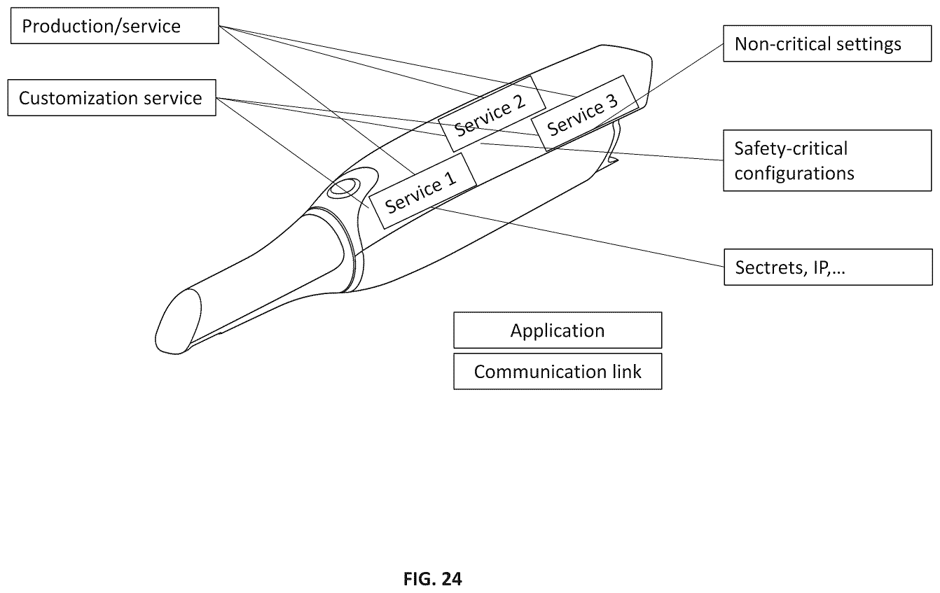

FIG. 25 is a schematic illustration of an intraoral scanning device service access control which is client specific. The invention addresses the implementation of such client specific intraoral scanning service access on an intraoral scanning device in an efficient manner.

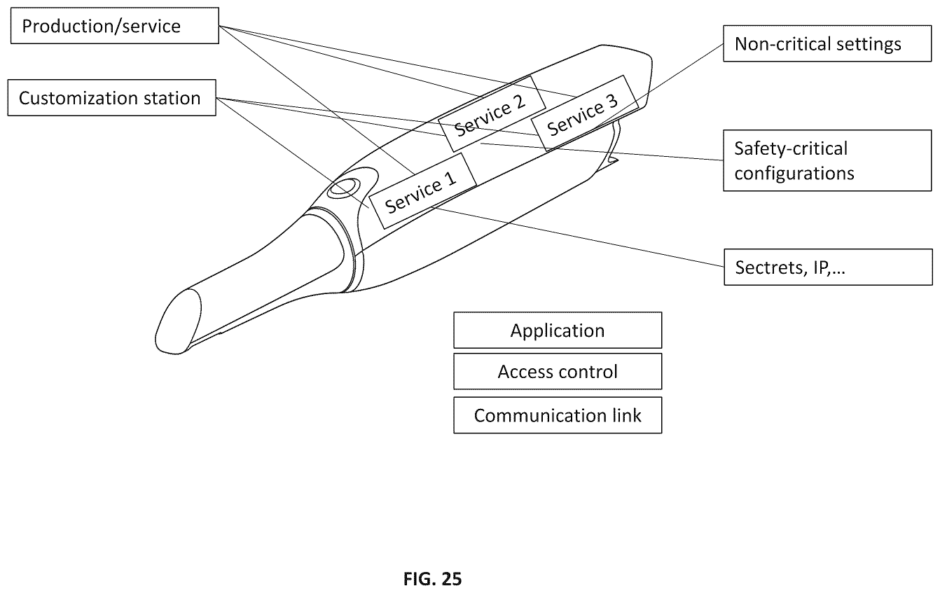

FIG. 26 is a block diagram of an example of an intraoral scanning device 10 configured to acquire intraoral scan data from a three-dimensional dental object 290. The intraoral scanning device 10 comprise a wireless interface 20 configured to exchange data from or to a client device 40, for example for receiving or transmitting customization data, updating data, debug data.

According to one example, the wireless interface 20 may be a Bluetooth interface, preferably a Bluetooth Low Energy (BTLE) interface, or a WIFI interface.

The intraoral scanning device 10 comprises a control unit 38 for controlling operation of the intraoral scanning device 10, with the control unit 38 acting on the processing unit 14 and the transceiver 28, and a memory 36 for storing data required for operation of the intraoral scanning device 10 and data required for operation of the wireless interface 20, such as pairing/network data.

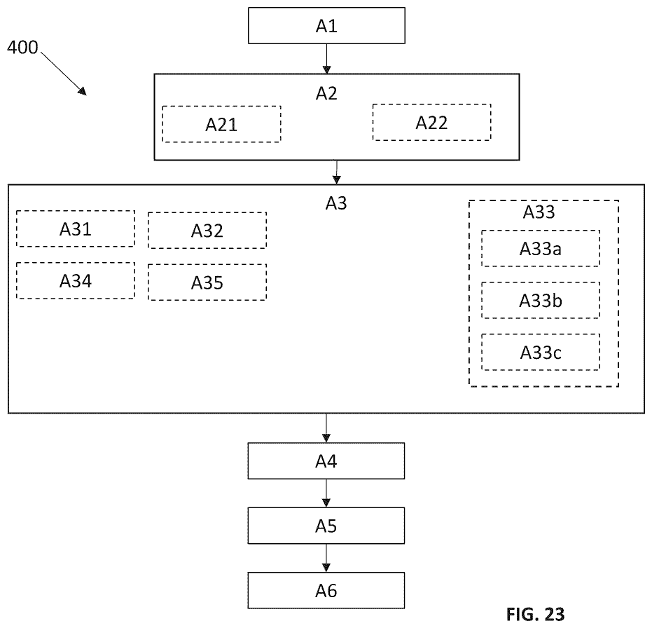

In the example of FIG. 24, the intraoral scanning device 10 comprises a light projector configured to emit light to a dental object, a camera configured to receive reflect light from the dental object, a processing unit 14 configured to process intraoral scan data and provide 2D image data and/or 3D image data to be forwarded via the wireless interface 20.

The intraoral scanning device service access control concept of the present disclosure includes the following main aspects: A plurality of intraoral scanning device services is defined, each having a certain criticality, and to each intraoral scanning device service a security level is assigned which is selected from a plurality of hierarchically structured security levels according to the criticality of the intraoral scanning device service.

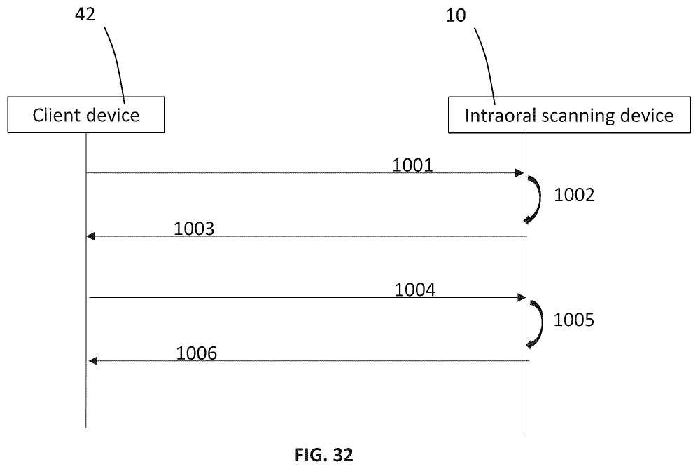

In FIG. 33 it is illustrated that the security levels are structured hierarchically in the sense that the access to the highest security level includes access to all lower security levels, i.e. access to the most critical services includes access to all lower security level services, down to the least critical services.

Further, a plurality of authorization methods is defined and at least one of the authorization methods is assigned to each of the security levels in such a manner that each of the authorization method(s) assigned to a certain security level is different to the authorization methods assigned to the other security levels, wherein each authorization method is for granting an authorization to a client to access intraoral scanning device service(s) assigned with the respective security level.

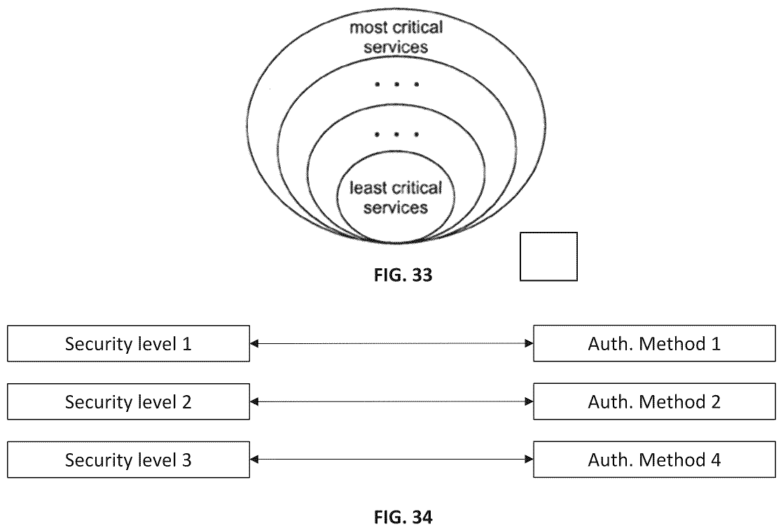

An example of such an assignment is schematically illustrated in FIG. 34, wherein a first security level, corresponding for example to a firmware update, is assigned with a first authorization method, such as authorization via an entity trusted by the intraoral scanning device, a second security level, such as corresponding to a customization process, is assigned with a second authorization method, for example authorization via a first user gesture, and a third security level, such as corresponding to a remote control access, is assigned with a third authorization method such as authorization via a second user gesture different from the first user gesture.

An authorization comprises at least a client authenticator and the highest security level granted to the client device, wherein a client privileged to access a certain security level (as a result of the respective authorization method) is also privileged to access all security levels below that level. At least one of the authorization methods may allow a user to grant authorizations autonomously without involvement of a third entity trusted by the intraoral scanning device: such autonomous authorization includes acting, in particular by a certain user gesture, on the intraoral scanning device itself or an external device communicating with the intraoral scanning device.

The granted authorizations are stored on the intraoral scanning device so as to allow enforcement of the access control during runtime on the intraoral scanning device, without the need for a third entity, such as a user account on a remote server.

Runtime enforcement of intraoral scanning device service access starts once the intraoral scanning device receives an intraoral scanning device service access request from a client device. Once the client device has been authenticated based on the stored client authenticator of the respective client device, the security level associated with the intraoral scanning device service requested by the client is compared to the highest security level granted to the client device according to the stored authorization of the client device, wherein, if the granted security level is not at least as high as the security level associated with the requested intraoral scanning device service, the intraoral scanning device rejects access to the requested intraoral scanning device service. If the granted security level is at least as high as the security level associated with the requested intraoral scanning device service, the intraoral scanning device typically will permit the access to the requested intraoral scanning device service.

Examples of authorization methods are as follows: authorization by the specific user gesture, authorization by predefined shared secrets, authorization via a third entity trusted by the intraoral scanning device, and authorization by default.

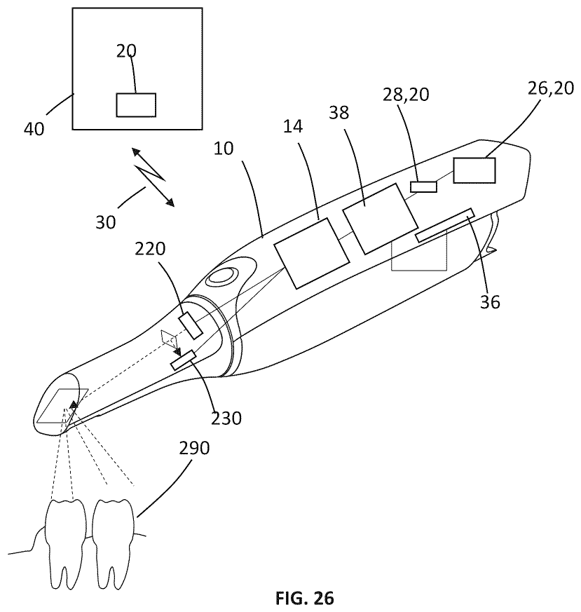

When using different user gestures for authorization, the user, for example, may use a first gesture to grant a full access to the intraoral scanning device to a customization station (the user in this case would be a dentist or an operator), whereas another gesture can be used to grant access to a restricted set of services of the intraoral scanning device, for example consisting only of customization data. The user may perform an authorization gesture in response to an authorization request from a client, with the intraoral scanning device informing the user about the reception of the authorization request. If the user decides to grant the requested authorization, the user will perform the respective gesture. Preferably, the user authenticates the requesting client device prior to authorizing it. A notification may indicate to the user which privileges are requested by the client device: such notification may occur via a user interface of the intraoral scanning device. The user interface may include a vibrator, one or more LED or a digital display. An illustration of such authorization method is illustrated in FIG. 27, the method involving a user 18, a client device 40 and an intraoral scanning device 10. The intraoral scanning device 10 receives 401 an authorization request from the client device 40, and the intraoral scanning device 10 transmits 402 an authorization request notification 402 to the user 18 via the client device 40. The user then authenticates 403 the requesting client device by forwarding a gesture 404 to the intraoral scanning device 10, and the intraoral scanning device grants 405 the authorization based on the gesture. The gesture may be an input to a user interface of the client device 40.

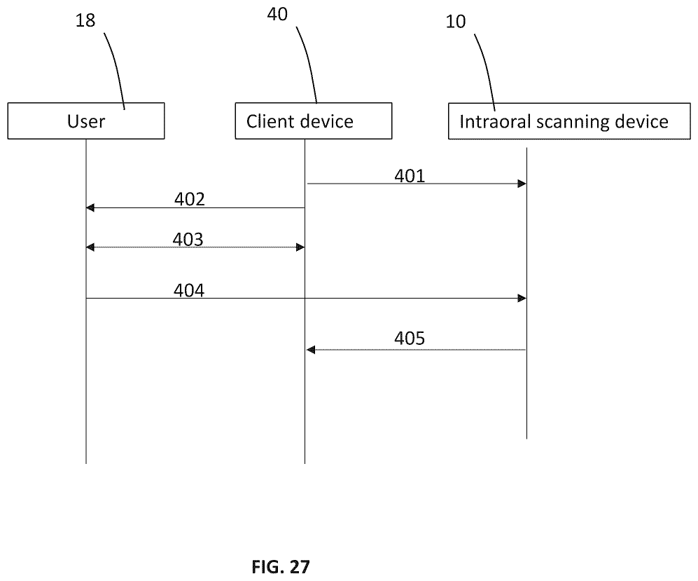

Alternatively, the user may first perform an authorization gesture 502, thereby bringing the intraoral scanning device into a state in which it accepts authorization requests from any client. Preferably, the intraoral scanning device 10 informs, upon entry into that state, the user which privileges will be assigned to client devices requesting 501 authorizations in this state. The user then may cause 503 the desired client device to send 504 an authorization request to the intraoral scanning device, whereupon the intraoral scanning device notifies 505 the user about successful authorization: such notification may inform the user to which client the authorization has been effectively granted, so that the user may withdraw the authorization in case he recognizes that the authorization was granted to a wrong client. An example of such authorization method is illustrated in FIG. 28.

According to another example, in case of a wireless connection, such as a connection using a Bluetooth protocol or a WIFI protocol, between the client device and the intraoral scanning device, the pairing/connection process (which authorizes a device wirelessly connected to n intraoral scanning device) and the authorization of the client device (i.e. the assignment of privileges to use a set of services on the intraoral scanning device) may be combined into one procedure as seen by the user. In such case, the same user gesture may be used at the same time for the pairing/connection process and for the assignment of privileges (i.e., for the authorization process). Alternatively, the pairing/connection gesture may be different from the authorization gestures.

According to one example, the authorization gesture may be performed on a user interface of the client device 40 or the intraoral scanning device 10. For example, a long press on a button and a short press on a button of the intraoral scanning device can be used as different gestures to grant different authorizations (i.e., to assign different sets of privileges).

According to another group of authorization methods, the authorization may comprise authorization by shared secretes, wherein a shared secret is associated with one of the security levels, with the shared secrets being stored on the intraoral scanning device and being provided to at least one client device, and wherein a client device is authorized with the requested security level if it presents a valid proof to the intraoral scanning device that it knows the shared secret. In this case, different sets of privileges (i.e., different authorizations) can be associated with different secret values stored in the intraoral scanning device, for example at the time of manufacturing. The problem of shared secret distribution to client devices can be solved in different ways, e.g., (1) if the client is under full control of the intraoral scanning device manufacturer (for example, it is a cloud service owned by the manufacturer), the shared secret can be directly provided to the client device; and (2) if the client is a customization station, the shared secret can be provided to it upon successful authentication and authorization of the customizer by the manufacturer. If the shared secrets are not unique to an intraoral scanning device, but the same for all devices (which is a weak solution from security point of view), then the secrets can be distributed together with the client installation package.

For example, in order to achieve full access to an intraoral scanning device, a customization station has to prove to the intraoral scanning device that it knows a first secret.

A client can prove to the intraoral scanning device that it knows a secret by using different methods, for example, the secret can be communicated in clear text via a communication channel that guarantees confidentiality (like an encrypted Bluetooth or WIFI link) or the client and the intraoral scanning device may use a cryptographic challenge-response protocol.

Another group of authorization methods is authorization via a trusted entity. In this case, an authorization service which is an entity trusted by the intraoral scanning device, is used to authorize intraoral scanning device client devices, wherein a client device that desires access to intraoral scanning device services requests the desired access from the authorization service, for example via a user log-in at the authorization service. If the authorization service decides to grant the requested authorization to the client device, it issues a token to the client device, which may contain the set of granted privileges. In order to obtain the requested intraoral scanning device service access, the client device then presents the token to the intraoral scanning device which, if it successfully authenticates the token as issued by the trusted authorization service, then grants the requested set of privileges to the client device.

Since such approach is susceptible to the replay attacks a more advanced alternative approach may be used, wherein the intraoral scanning device issues a ‘token’ to client device. The client device provides the token to the authorization service, which (1) signs the token (so called nonce); and (2) creates and signs a shared key to be used by the client device and the intraoral scanning device (i.e., establishes a trust relation between them). Then the authorization service distributes in a confidential manner the signed token and the key to the client device and the intraoral scanning device. Usually, this is done through the client device. Thus, two encrypted copies of signed token-key pair are provided first to the client device. One copy is encrypted such that only the client device can decrypt it.

The other copy is encrypted such that only the intraoral scanning device can decrypt it. The client device extracts its copy for itself and forwards the other copy to the intraoral scanning device. The intraoral scanning device verifies the authorization service signature and if it is valid, accepts the shared secret (which can be used as the client authenticator). Same is done by the client, if the confidentiality and integrity of the channel between the client and the authorization service are not guaranteed.

For example, if an authorization service can authenticate a person (typically via a user log-in) as a dentist who is authorized to perform customization of a particular intraoral scanning device, the authorization service issues to that person a first token granting full access to the intraoral scanning device. If the authorization service can authenticate a person as the owner/end-user of an intraoral scanning device (via a user log-in into the authorization service), the authorization service issues to that person a second token granting a limited set of privileges which, for example, is only sufficient to send remote control, commands to the intraoral scanning device, but not to change its customization parameters.

The trusted relation between an authorization service and the intraoral scanning device can be established, for example, based on symmetric cryptography using a secret which is pre-shared between the authorization service and the intraoral scanning device (for example, the shared secret may be provided at the time of manufacturing of the intraoral scanning device): preferably, the shared secret is unique for each intraoral scanning device.

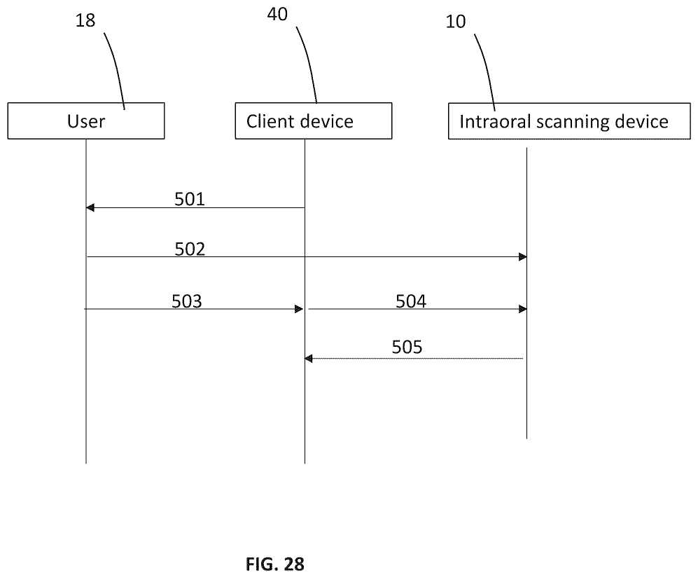

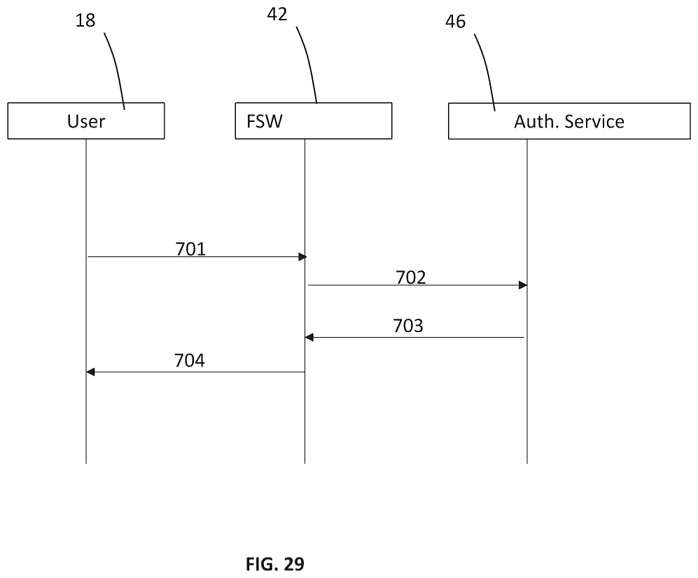

An example of an establishment of a trusted relation is illustrated in FIGS. 29 and 30, wherein the steps shown in FIG. 29 precede the steps shown in FIG. 30, with example involving a dentist 18, a client device, such as customization station 42, an intraoral scanning device 10 and a manufacturer authorization service 46. In FIG. 29, the dentist 18 is logging 701 onto the client device 42 or an application installed on the client device, and the username and password are being forwarded 702 to the intraoral scanning device 10 via the client device 42, and the intraoral scanning device 10 forwards 703 an access token to the client device 42, and the access token includes information which relates to whether the dentist is successfully authorized. The client device 42 informs 704 the dentist whether the login is ok.

The client authenticates itself with the authorization service 46 by the steps shown in FIG. 29 prior to the message exchange shown in FIG. 30. In step 801 the client device 42 requests authorization form the intraoral scanning device 10. In step 802 a nonce and the intraoral scanning device ID are sent from the intraoral scanning device 10 to the client device 42: this message can be encrypted with the key pre-shared between the intraoral scanning device 10 and the authorization service 46, which key can be a shared or a public key. In step 803 the client device sends authorization request including the nonce, the intraoral scanning device ID, the client device ID and the requested security level to the authorization service 46, whereupon the authorization service 46 checks the client's access rights (step 804) and sends an authorization grant including the client authenticator to the client device 42 (step 805). The channel between the client device 42 and the authorization service 46 is assumed to be confidential and integer. In step 806 the authorization service 46 sends an authorization grant conformation message to the intraoral scanning device, the message including the nonce, the intraoral scanning device ID, the client device ID, the requested security level, and the client authenticator. The message is authenticated by authorization service 46 either using the key pre-shared between the intraoral scanning device and the authorization service 46 or by private key of the authorization service 46. If confidentiality of the channel is not guaranteed, the message can be encrypted with the key pre-shared between the intraoral scanning device and the authorization service 46 or with a temporary key provided by the intraoral scanning device within the message of step 802 (the messages of step 802 in this case has to be also encrypted). The message of step 806 can be sent to intraoral scanning device 10 ‘directly’ or via the client device 42. The client device ID, security level, and the authenticator (shared key) may be stored 807 on the intraoral scanning device 10.

Thus, the trusted relation may be established based on public key cryptography, wherein the authorization service possesses a private key and the intraoral scanning device knows the corresponding public key (which may be stored, for example, within the intraoral scanning device in a write-protected memory): preferably, the public/private key pair is unique for the intraoral scanning device: alternatively, the public/private key pair can be the same for all or for a group of intraoral scanning devices.

The token may be a digital certificate issued by the authorization service to the client, wherein the digital certificate may be signed with the private key of the authorization service and wherein the intraoral scanning device may use the public key to validate the signature of the certificate in order to verify the certificate. The intraoral scanning device may install the certificate, when successfully verified, in its write-protected memory. The certificate may be of a standard format and may contain an authenticator of the client device to which the certificate is issued, a client public key generated and provided by the client device to the authorization service, and the security levels granted to the client device. The client private key is stored by the client device as a secret. Later, the intraoral scanning device can use the client public key to authenticate the client device and/or it may use it for any other purposes requiring cryptographically protected confidentiality and integrity of communication, such as for key distribution.

The authorization service may be provided via a communication network, such as the internet: in particular, it may be implemented on a server run by the manufacturer of the intraoral scanning device.

In addition to the authorization methods described so far, the authorization may occur by default, wherein the intraoral scanning device unconditionally assigns a given minimum security level to any client requesting authorization: this applies to non-critical intraoral scanning device services, such as settings that relates not to acquiring of intraoral scan data and processing of the intraoral scan data.

As already mentioned above, the result of a successful authorization is a client authenticator and the highest security level granted to the client. Preferably, the client authenticator contains a secret shared between the client and the intraoral scanning device. According to one example, the shared secret may be established by a cryptographic protocol, such as Diffie-Hellman. Alternatively, the shared secret (i.e. a shared key) may be established between the client device and the intraoral scanning device through the authorization service during the authorization process as exemplified in the message sequence charts in FIGS. 27 and 28. According to a further alternative, if the underlying communication channel ensures confidentiality and integrity, the shared secret may be generated by the client device and is transmitted in clear to the intraoral scanning device (or vice versa). The secret can be a shared key or a private/public key pair.

Later, the shared secret of the client authenticator (which shared secret is to be distinguished from the shared secrets mentioned with regard to the authorization methods) may be used to achieve end-to-end security (i.e. confidentiality and integrity) of the communication between the client device and the intraoral scanning device, if the underlying communication channel is going through untrusted entities, such as the internet (as would be the case for example, in remote customization update, firmware update, debugging of the intraoral scanning device).

Similar methods and mechanisms as described above may be used to revoke a previously granted authorization to intraoral scanning device services.

The intraoral scanning device starts to accept service requests from a client device only if it is able to successfully authenticate the client.

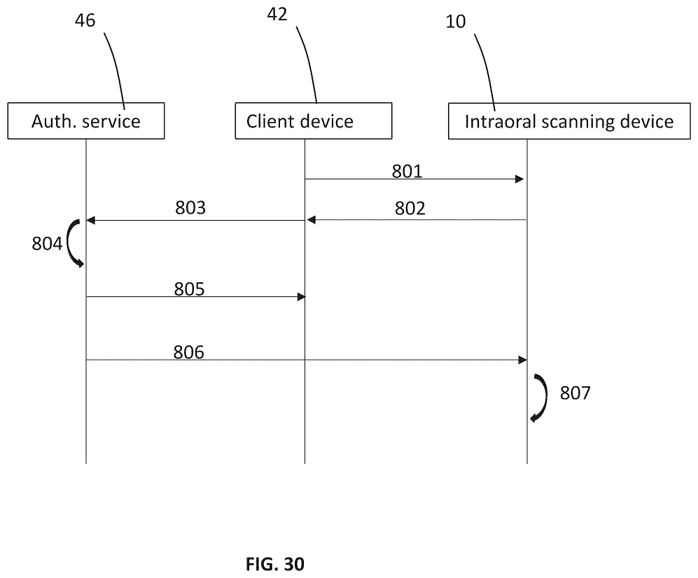

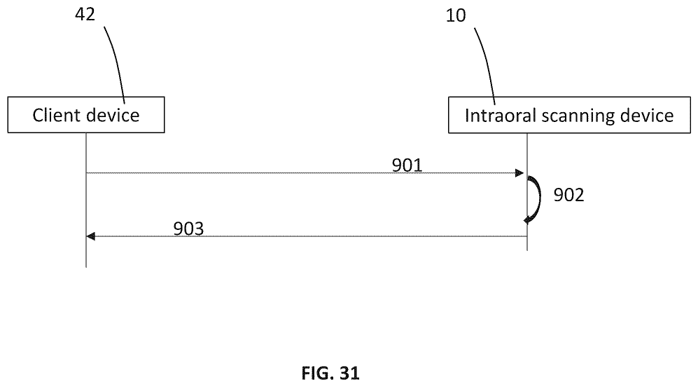

If the underlying communication channel between the client device and the intraoral scanning device guarantees confidentiality and integrity, the shared secret established during authorization may be transmitted 901 in clear text from the client to the intraoral scanning device so as to authenticate 902 the client device. An authentication response 903 is forwarded to the client device 42 informing whether the authentication is successful. An example of such authentication is illustrated in FIG. 31, involving a client device 42 and an intraoral scanning device 10. If the communication channel between the client device 42 and the intraoral scanning device 10 guarantees integrity but not confidentiality, the shared secret established during authorization is used in a cryptographic challenge-response protocol. An example of such authentication is illustrated in FIG. 32. In step 1001, the client device requests 1001 a challenge to the intraoral scanning device 10, and the intraoral scanning device 10 generates random number, i.e., a challenge, which is then forwarded 1003 to the client device 42. The client device replies 1004 to the challenge by forwarding random generated number which is verified 1005 by the intraoral scanning device 10 and informs 1006 if the verification is successful.

In both cases, the client authentication needs to be performed only once (for example, upon link establishment), while achieving permanent authentication. However, if the communication channel between the client device and the intraoral scanning device does not guarantee integrity, every single service request by a client has to be authenticated (i.e., there is only a one-time authentication): this may occur by known cryptographic techniques such as message authentication codes (MAC) or digital signatures. By “permanent” it is not necessarily meant that the authentication is done only once and forever. Rather, the authentication is performed in the beginning of each session (assuming the confidentiality and integrity of the channel). For example, it may be performed every time a smart phone reconnects to the intraoral scanning device via Bluetooth or WIFI, but it can be performed even more often, for example, for every logically self-contained interaction on application level (i.e., session).

Certain (non-critical) service requests may not require a prior client authentication and therefore would be always accepted by the intraoral scanning device (this corresponds to the above-mentioned “authentication by default”).

As already mentioned above, once the intraoral scanning device has successfully authenticated the client device and has found that the security level granted to the client device is at least as high as the security level associated with the service request, the intraoral scanning device typically permits the access to the requested intraoral scanning device service.

Preferably, the security levels are represented by the numerical values, with the order of the numerical values being correlated with the hierarchy of the security levels. For example, the security level may be the higher the numerical value representing the security level is. According to one example, a call dispatching table may be stored on the intraoral scanning device for assigning each intraoral scanning device service callable by a client device to one of the security levels.

According to one example, the security levels (and thus the intraoral scanning device services associated with the security levels) accessible by a certain client device may be expressed by white-listing (listing all services/security levels accessible by the client) or by black-listing (i.e., listing all services/security levels which are not accessible by the client device).

According to one example, the client devices may be grouped based on the highest security level accessible by the client device, with each group being assigned with the respective highest security level accessible by the client devices of the group, wherein the intraoral scanning device permits access to the requested intraoral scanning device service if the security level associated with the requested intraoral scanning device service is not higher than the security level of the group of the client devices, otherwise it rejects the access.

The client device may comprise devices, such as customization stations, clinic computer, tablets, test systems, repair, and service stations, as well as application programs running on such devices.

The present disclosure offers several benefits: for example, since the authentication methods include authentication by user gesture, the user keeps control of client device access to his intraoral scanning device. Further, the present disclosure protects the intraoral scanning device from man-in-the-middle attacks during pairing, while nevertheless the access control may be implemented in a manner that requires only little resources of the intraoral scanning device.

Client Device with Certificate and Related Method:

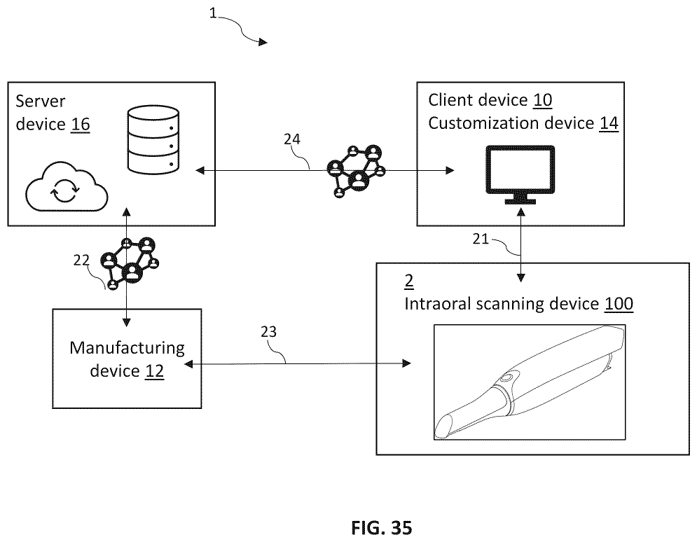

FIG. 35 schematically illustrates an exemplary architecture according to this disclosure with exemplary devices that may be used for manufacturing, maintenance, and/or operating an intraoral scanning device 2. FIG. 35 shows an exemplary system 1 and an intraoral scanning device 2. The system 1 may comprise one or more of a manufacturing device 12, a client device 10, and a server device 16 for manufacturing, maintenance, and/or operating the intraoral scanning device 2 in connection with intraoral scanning session (such as for customizing the intraoral scanning device and/or for updating an intraoral scanning device parameter).

The client device 10 may be configured to perform any acts of the method disclosed herein. The client device 10 may comprise processing elements (such as a processor and a memory) configured to perform any of the steps of the method disclosed herein. The intraoral scanning device 2 may be configured to acquire intraoral scan data from a three-dimensional dental object during a scanning session. The intraoral scanning device 2 may be configured to communicate with the client device 10 using e.g., a communication link 21. The communication link 21 may be a wireless communication link. The communication link 21 may be a single hop communication link or a multi-hop communication link. The wireless communication link may be carried over a short-range communication system, such as Bluetooth, Bluetooth low energy, IEEE 802.11. The intraoral scanning device 2 may be configured to receive an intraoral scanning device certificate from the manufacturing device 12 via communication link 23 and to store the intraoral scanning device certificate in a memory unit comprised in the intraoral scanning device 2. Alternatively, or additionally, the manufacturing device 12 may store the intraoral scanning device certificate in the memory unit of the intraoral scanning device. The intraoral scanning device 2 may configured to connect to the client device 10 over a network. The client device 10 may permit remote customization of the intraoral scanning device 2, where a dispenser connects to the intraoral scanning device via the client device 10. The client device 10 may comprise a computing device acting as a client, such as a customization device 14 (e.g., a handheld device, a relay, a tablet, a personal computer, and/or USB dongle plugged in a personal computer). The client device 10 may be configured to communicate with the server device 16 via a communication link 24, such as a bidirectional communication link. The communication link 24 may be a wireless communication link. The communication link 24 may comprise a network, such as the Internet. The client device 10 may be configured to communicate with the server device 16 for maintenance, and update purposes. The server device 16 may comprise a computing device configured to act as a server, i.e., to serve requests from the client device 10 and/or from the intraoral scanning device 2. The server device 16 may be controlled by the intraoral scanning device manufacturer. The server device 16 may be configured to communicate with the manufacturing device 12 via a communication link 22 for manufacturing maintenance, and/or operational purposes. The server device 16 and the manufacturing device 12 may be co-located and/or form one entity for manufacturing maintenance, and/or operational purposes of the intraoral scanning device 2.

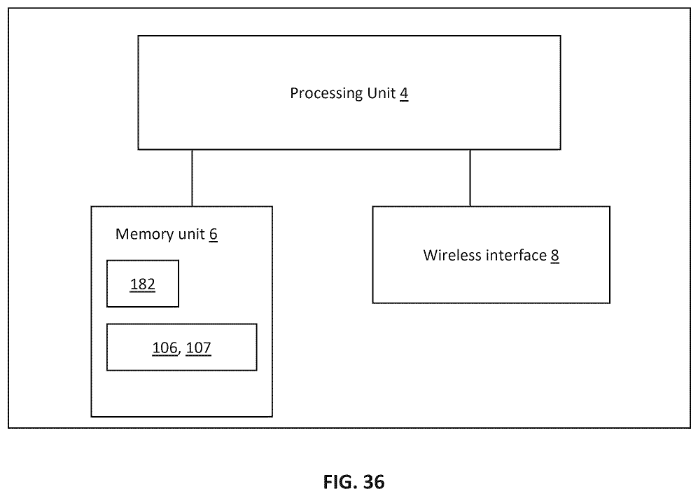

FIG. 36 schematically illustrates an exemplary client device 10. The client device 10 comprises a processing unit 4, a memory unit 6 and a wireless interface 8. The wireless interface 8 comprises a wireless transceiver, e.g., configured for wireless communication at frequencies in the range from 2.4 to 2.5 GHZ. The wireless interface 8 is configured for communication, such as wired and/or wireless communication, with an intraoral scanning device 2 and/or a server device. The memory unit 6 has a client device key 182 and a client device certificate 106, 107 stored thereon. The processing unit 4 is configured to receive a connection response comprising an intraoral scanning device identifier via the wireless interface 8 and optionally to obtain a session identifier, e.g., as part of the connection response. A connection response including both the session identifier and the intraoral scanning device identifier reduces the risk of intervention from an attacker. Further, the number of connection responses from an intraoral scanning device is reduced, thereby reducing power consumption in the intraoral scanning device. The processing unit 4 is configured to generate one or more keys including a certificate key based on the intraoral scanning device identifier and/or the session identifier. The processing unit 4 is configured to generate one or more keys including a certificate key optionally based on the client device key. In the illustrated client device 10, the certificate key is generated by performing one or more hash functions. For example, the certificate key, C_KEY, may be given as:

$$C\_KEY = hash ( CS , C\_VAL ) ,$$

where hash is a hash function, CS is a common secret and C_VAL is a certificate value, e.g., a predefined value or string, such as “certificate”. In the exemplary client device 10, the common secret, CS, is based on the intraoral scanning device key and the session identifier, e.g., given as:

$$CS = hash ( IOS\_KEY , S\_ID ) ,$$

where hash is a hash function, IOS_KEY is the intraoral scanning device key and S_ID is the session identifier. The intraoral scanning device key, IOS_KEY, is based on the intraoral scanning device identifier and the client device key, e.g., given as:

$$IOS\_KEY = hash ( IOS\_ID , CD\_KEY ) ,$$

where hash is a hash function, IOS_ID is the intraoral scanning device identifier and CD_KEY is the client device key.

The certificate value may be a predefined value or string, such as “certificate”. Generating the certificate key may comprise performing a hash function on the common secret and/or the certificate value. For example, the certificate key, C_KEY, may be given as:

$$C\_KEY = hash ( CS , C\_VAL ) ,$$

where hash is a hash function, CS is the common secret and C_VAL is the certificate value.

The processing unit 4 is configured to obtain an authentication message based on the certificate key and the client device certificate. To obtain the authentication message comprises to generate an encrypted client device certificate by encrypting the client device certificate with the certificate key and to include the encrypted client device certificate in the authentication message. To obtain the authentication message comprises to include an authentication key identifier and/or an authentication type identifier in the authentication message. The authentication key identifier is a copy of or at least indicative of the client device key identifier. The authentication type identifier is a copy of or at least indicative of the client device type identifier. The use of authentication identifier(s), such as authentication key identifier and/or authentication type identifier in the authentication message enables an intraoral scanning device to select the correct keying material for decrypting the encrypted client device certificate and/or check whether the authentication message is generated by an outdated client device. Further, the processing unit 4 is configured to transmit the authentication message to the intraoral scanning device via the wireless interface 8.

In the exemplary processing unit 4, to generate one or more keys comprises to generate a session key based on the intraoral scanning device identifier, the session identifier, and the client device key, and wherein the processing unit is optionally configured to transmit the session key to a customization device. When the client device 10 comprises the customization device, the session key is used for data communication with the intraoral scanning device.

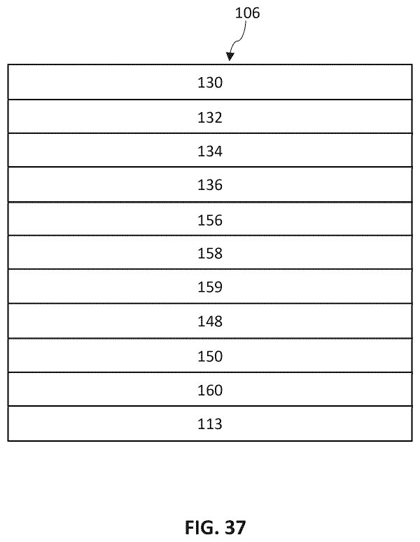

FIG. 37 schematically illustrates an exemplary client device certificate 106. The client device certificate 106 comprises a client device identifier 158 and a client device key identifier 159. The client device identifier 158 enables an intraoral scanning device to check if the client device has been black-listed. The client device key identifier 159 is indicative of the client device key (stored in the memory unit) used for generating the certificate key. The client device key identifier 159 of the client device certificate enables an intraoral scanning device to check the validity of the authentication key identifier of the authentication message.

The client device certificate 106 comprises a digital signature 113 and/or a MAC. The digital signature 113 enables a proof or verification of authenticity of the client device certificate 106, such as verification of the signer legitimacy (e.g., whether the signer is a legitimate manufacturing device). The digital signature 113 is generated during manufacture, e.g., using a device family private key during manufacturing of the client device. The client device 10 or the processing unit 4 may verify the digital signature 113 when receiving the client device certificate 100 comprising the digital signature 113. The digital signature 113 is verifiable by the client device 10 and/or an intraoral scanning device using a corresponding device family public key, e.g., selected according to the certificate type identifier. If the digital signature 113 is not successfully verified using the alleged public key, the client device 10 may abort normal operation.

The client device certificate 106 comprises a certificate type identifier 130. The certificate type identifier 130 indicates that the client device certificate 106 is a client device certificate, e.g., selected amongst a variety of certificate types, such as an intraoral scanning device family certificate type, an intraoral scanning device certificate type, a firmware certificate type, an access right certificate type, and a client device certificate type. The certificate type identifier 130 may be used to enable an intraoral scanning device 2 to identify what type of certificate it receives, stores, authenticates and/or retrieves. The client device certificate 106 may comprise a version identifier 132 which indicates a data format version of the client device certificate 106. An intraoral scanning device 2 may use the certificate type identifier 130 and/or the version identifier 132 to determine what type of data the client device certificate 106 comprises and/or what type of data is comprised in a field of the client device certificate 106. For example, an intraoral scanning device may determine based on the certificate type identifier 130 and/or version identifier 132 what field of the client device certificate comprises a digital signature 113, and which public key from a plurality of public keys is to be used to verify the digital signature 113. It may be envisaged that there is a one-to-one mapping between the certificate type identifier 130 and the public-private key pair used for generating the digital signature 113. The intraoral scanning device certificate 106 may comprise a length identifier 134 that indicates the length of the client device certificate 106.

The client device certificate 106 optionally comprises a signing device identifier 136. The signing device identifier 136 refers to a unique identifier identifying the device (such as an integrated circuit card, a smart card, a hardware security module comprised in or connected to a manufacturing device) that has signed the client device certificate 106. The signing device identifier 136 may for example comprise a medium access control, MAC, address of the signing device and/or a serial number. The signing device identifier 136 allows for example an intraoral scanning device 2 to determine whether the signing device of the client device certificate is e.g., black-listed or not, and thus to reject client device certificates 106 signed by a signing device that is black-listed.

The client device certificate 106 optionally comprises one or more hardware identifiers including a first hardware identifier 148 and/or a second hardware identifier 150. The hardware identifiers 148, 150 may respectively identify a piece of hardware comprised in the client device 10, such as a processing unit 4 or a radio chip comprised in the wireless interface 4. The first hardware identifier 148 and/or the second hardware identifier 150 may also be stored in a register of the piece of hardware comprised in the client device 10 during manufacturing of the piece of hardware. The first hardware identifier 148 and/or the second hardware identifier 150 may comprise a serial number, a medium access control, MAC, address, a chip identifier, or any combination thereof. For example, the first hardware identifier 148 may provide a first client device specific value present in a register of a hardware module (e.g., the processing unit or the radio chip) of the client device 10 while the second hardware identifier may provide a second client device specific value present in a register of a hardware module of the client device 10.

The client device certificate 106 comprises a client device type identifier. 156. The client device type identifier 156 indicates a type of the client device amongst a variety of client device types, such as a model, category, or type of client devices, such as a customization type, e.g., a tablet product model, category or type for customizing the intraoral scanning device, a USB dongle product model, category or type for customizing the intraoral scanning device. Optionally, the client device certificate 106 comprises a Bluetooth address 160 or at least part thereof, e.g., assigned by the manufacturer during manufacture.

Addition of one or more fields and/or identifiers to the client device certificate is contemplated e.g., for a second generation client device certificate.

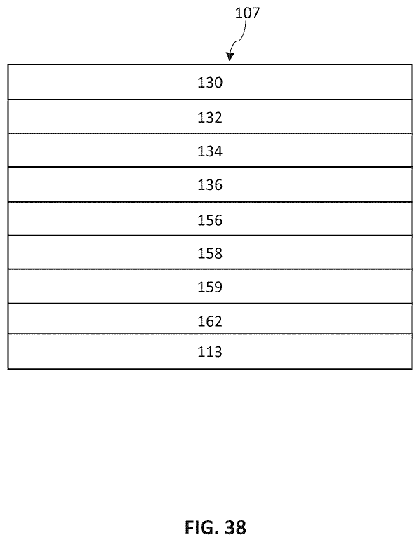

FIG. 38 schematically illustrates an exemplary client device certificate 107. The client device certificate 107 comprises certificate type identifier 130, optional version identifier 132, optional length identifier 134 and optional signing device identifier 136 as described above for client device certificate 106. The client device certificate 107 comprises client device type identifier 156, client device identifier 158, client device key identifier 159, and a user identifier 162. The user identifier 162 may be a in the form of a username. Client device certificate 107 with a user identifier 162 may facilitate the use of a generic device, such as a tablet computer, as a client device, e.g., by implementing a user verification/key generation/certificate encryption/decryption at a remote server device, such as server device 16 controlled by intraoral scanning device manufacturer.

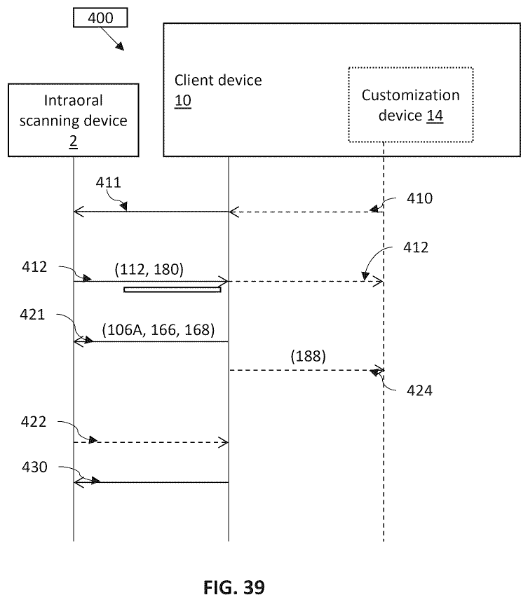

FIG. 39 schematically illustrates an exemplary signaling diagram 400 involving an intraoral scanning device 2 and a client device 10. The client device 10 may comprise a customization device 14 or be connected to a customization device 14. The client device 10 transmits a connection request or message 411 to intraoral scanning device 2. When the client device 10 comprises a customization device 14, the customization device 14 may generate a connection request 410, which is transmitted by the client device 10 as connection request 411. When the client device 10 is connected to a customization device 14, the customization device 14 may generate a connection request 410, which is forwarded by the client device 10 as connection request 411. The intraoral scanning device 2 returns a connection response 412 which is received by the client device 10. The client device 10 may forward the connection response 412 to the customization device 14. The connection response 412 comprises an intraoral scanning device identifier 112 and/or a session identifier 180. The client device 10 generates one or more keys including a certificate key based on the intraoral scanning device identifier 112 and/or session identifier 180 received in the connection response and the client device key 182 stored in the memory unit. The client device 10 obtains and transmits authentication message 421 to the intraoral scanning device 2 based on the certificate key and the client device certificate 106. The authentication message 421 comprises encrypted client device certificate 106A.

The encrypted client device certificate 106A is generated by encrypting the client device certificate 106 with the certificate key. The authentication message 421 comprises an authentication key identifier 166 indicative of the client device key 182 and/or authentication type identifier 168 indicative of the client device type identifier 156. Upon successful authentication of the authentication message 421 and/or verification, the client device 10 may receive an authentication response 422 from the intraoral scanning device 2. The client device 10 may forward the authentication response 422 to customization device 14. The communication channel is now open and secure. The client device 10 or customization device 14 via the client device 10 may send intraoral scanning device data 430 to the intraoral scanning device 2. Intraoral scanning device data 430 may comprise one or more of firmware, customization data, and/or intraoral scanning device operating parameters. Intraoral scanning device operation parameters may comprise volume control parameters, mode and/or program control parameters. Firmware may refer to a computer program provided by the intraoral scanning device manufacturer, and to be installed on the intraoral scanning device 2 to control the intraoral scanning device 2. Firmware is for example to be installed to upgrade the operations and capabilities of the intraoral scanning device 2. The client device 10 may transmit an authentication message 424 comprising a session key 188 to the customization device 14. The session key may be used for secure data communication 430 with the intraoral scanning device 2

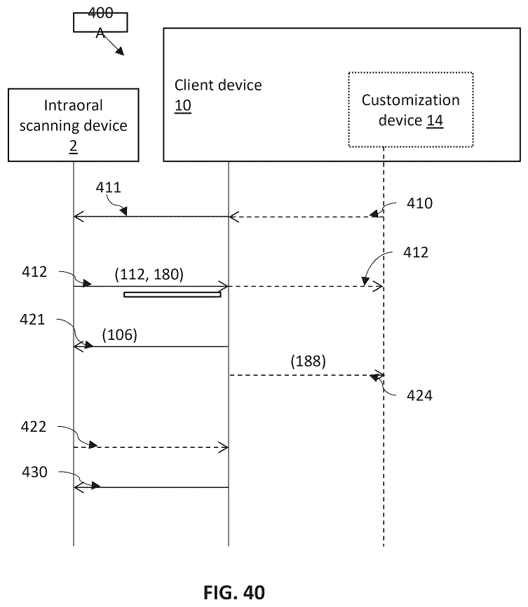

FIG. 40 schematically illustrates an exemplary signaling diagram 400A where the client device certificate 106 is included in the authentication message 421.

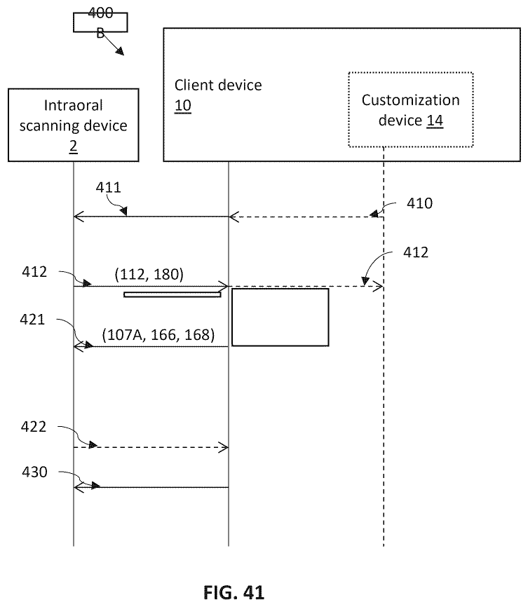

FIG. 41 schematically illustrates an exemplary signaling diagram 400B where the encrypted client device certificate 107A is included in the authentication message 421.

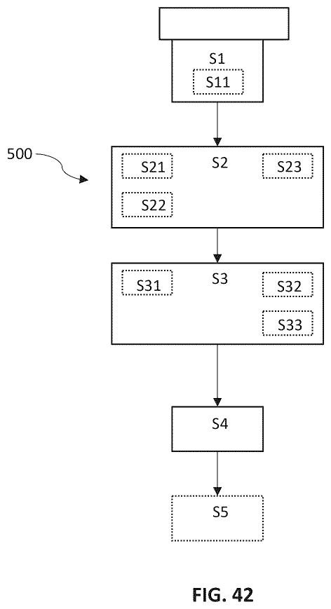

FIG. 42 schematically illustrates a flowchart of an exemplary method 500 of operating a client device for intraoral scanning device communication. The client device comprises a memory unit having a client device key and a client device certificate stored thereon. The method comprises receiving S1 a connection response comprising an intraoral scanning device identifier via the wireless interface: generating S2 one or more keys including a certificate key based on the intraoral scanning device identifier and the client device key: obtaining S3 an authentication message based on the certificate key and the client device certificate; and transmitting S4 the authentication message via the wireless interface. Obtaining S3 the authentication message comprises generating S31 an encrypted client device certificate by encrypting the client device certificate with the certificate key and including the encrypted client device certificate in the authentication message. The method 500 comprises obtaining S11 a session identifier as part of the connection response. Generating S2 one or more keys comprises generating S21 an intraoral scanning device key based on the intraoral scanning device identifier and the client device key and generating S22 a common secret based on the intraoral scanning device key and the session identifier. The certificate key is based on the common secret and a certificate value. Generating S2 one or more keys optionally comprises generating S23 a session key based on the intraoral scanning device identifier, the session identifier, and the client device key. The method may comprise transmitting S5 the session key to a customization device. In the method 500, the session key is based on the common secret and a session value. Obtaining S3 the authentication message comprises including S32 an authentication key identifier indicative of the client device key in the authentication message and/or including S33 an authentication type identifier in the authentication message.

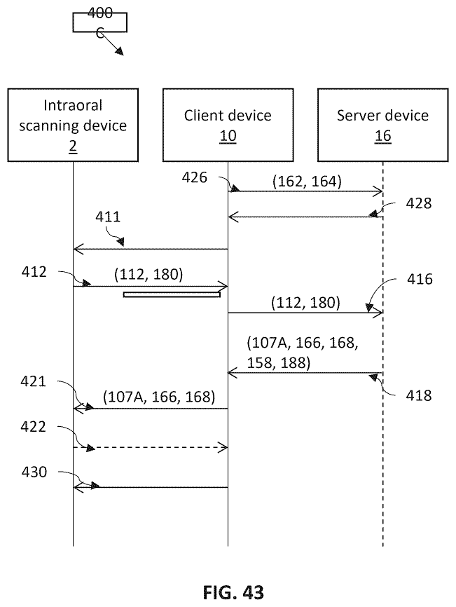

FIG. 43 schematically illustrates an exemplary signaling diagram 400C where obtaining the authentication message comprises obtaining an encrypted client device certificate from a server device. The client device 10 transmits a certificate request 416 to a server device 16. The certificate request 416 comprises the intraoral scanning device identifier 112 and the session identifier 180. The server device 16 obtains the client device certificate 107 from a memory unit thereof, calculates certificate key and session key based on the intraoral scanning device identifier 112 and the session identifier 180 and encrypts the client device certificate 107. The server device generates and transmits certificate response 418 to the client device 10. The certificate response 418 includes encrypted client device certificate 107A, authentication type identifier 168, authentication key identifier 166, client device identifier 158 and session key 188. The client device receives the certificate response 418 with the encrypted client device certificate 107A and includes the encrypted client device certificate in the authentication message. Thus, obtaining an encrypted client device certificate may comprise receiving a certificate response comprising the encrypted client device certificate from a server device. Optionally, the client device may, as illustrated in FIG. 43 be configured to perform a login procedure comprising transmitting a login request 426 comprising user identifier 162 and password 164. The server device 16 verifies the login request 426 and returns with login response 428 upon accept.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to a component/unit/element in the singular is not intended to mean “one and only one” unless explicitly so stated, but rather “one or more.” A claim may refer to any of the preceding claims, and “any” is understood to mean “any one or more” of the preceding claims.

It is Intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e., to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element but an intervening element may also be present, unless expressly stated otherwise. Furthermore, “connected” or “coupled” as used herein may include wirelessly connected or coupled. As used herein, the term “and/o” includes any and all combinations of one or more of the associated listed items. The step of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure, or characteristic described in connection with the embodiment is included in at least one embodiment of the disclosure. Furthermore, the particular features, structures or characteristics may be combined as suitable in one or more embodiments of the disclosure The previous description is provided to enable any person skilled in the art to practice the various aspects described herein. Various modifications to these aspects will be readily apparent to those skilled in the art, and the generic principles defined herein may be applied to other aspects

The claims are not intended to be limited to the aspects shown herein but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

### First Item Set:

Item 1. A handheld intraoral scanning device (10) configured to acquire intraoral scan data from a three-dimensional dental object during a scanning session, the handheld intraoral scanning device comprising
a processing unit (2) configured to process intraoral scan data of a patient and provide 3D image data;
a wireless interface (4) configured to transmitting the 3D image data, and
a memory (3):
   characterized in that the processing unit (202) is configured to:
receive a session request (301) for a session via the wireless interface (204), wherein the session request (301) includes a digital signature;
authenticate the digital signature for initiating the session;
obtain and store a session key;
sign the session key by an intraoral scanning device key, wherein the intraoral scanning device key is stored in a permanent memory of the intraoral scanning device (101);
send a session response (302) comprising the signed session key; and
receive session data (303) in the session via the wireless interface (204), and wherein the session data (303) corresponds to customization data for the handheld intraoral scanning device, handheld intraoral scanning device operating parameters and/or firmware data for the handheld intraoral scanning device.

Item 2. An intraoral scanning device according to item 1, wherein the session key is encrypted based on the intraoral scanning device key, and where the session response includes the encrypted session key.

Item 3. An intraoral scanning device according to any of the preceding items, wherein the processing unit (202) is configured to receive a client signed session key via the wireless interface (204), and decrypt the received session data (303) in the session.

Item 4. An intraoral scanning device according to any of the preceding items, wherein the processing unit (202) is configured to verify integrity of the session data (303).

Item 5. An intraoral scanning device according to any of the preceding items, wherein the processing unit (202) is configured to terminate the session if integrity of the session data (303) is corrupted.

Item 6. An intraoral scanning device according to any of the preceding items, wherein the session data (303) comprises a message authentication code, and wherein to verify integrity of the session data (303) comprises to verify the message authentication code with the stored session key.

Item 7. An intraoral scanning device according to any of the preceding items, wherein the session data (303) comprises a digital signature, and wherein to verify integrity of the session data (303) comprises verifying the digital signature.

Item 8. An intraoral scanning device according to any of items 2 to 7, wherein the processing unit (202) is configured to:
decrypt the session data (303) with the session key, and
store at least part of decrypted session data in the memory unit (203).

Item 9. An intraoral scanning device according to item 8, wherein the processing unit (202) is configured to terminate the session if decryption of the session data (303) fails.

Item 10. An intraoral scanning device according to any of the preceding items, wherein the session data (303) comprises customization data, intraoral scanning device operating parameters and/or firmware data.

Item 11. An intraoral scanning device according to any of the preceding items, wherein the processing unit (202) is configured to process intraoral scan data of a patient and provide 2D image data and/or 3D image data according to the received session data (303).

Item 12. An intraoral scanning device according to any of the preceding items, wherein the session key is a symmetric key.

Item 13. An intraoral scanning device according to any of the preceding items, wherein to obtain the session key comprises to generate a random or pseudo-random number with a number generator contained in the intraoral scanning device.

Item 14. An intraoral scanning device according to any of the preceding items, wherein the intraoral scanning device key is a symmetric key or a public key of a private-public key pair.

Item 15. An intraoral scanning device according to any of the preceding items, wherein the processing unit (202) is configured to send an intraoral scanning device identifier in the session response (302).

Item 16. Method for communication with an intraoral scanning device (101) for acquiring intraoral scan data from a three-dimensional dental object during a scanning session comprising a processing unit (202) configured to process intraoral scan data of a patient and provide 2D image data and/or 3D image data, a memory unit (203), and a wireless interface (204), the method comprising:
receiving a session request (301) for a session via the wireless interface (204);
obtaining and storing a session key;
signing the session key by an intraoral scanning device key, wherein the intraoral scanning device key is stored in a permanent memory of the intraoral scanning device;
sending a session response (302) comprising the signed session key; and
receiving session data (303) in the session via the wireless interface (204).

Item 17. A method according to item 16, the method comprising verifying integrity of the session data (303).

Item 18. a method according to item 17, the method comprising terminating the session if integrity of the session data (303) is corrupted.

Item 19. a method according to any of items 16 to 18, the method comprising:
decrypting the session data (303) with the session key, and
storing at least part of decrypted session data in the memory unit (203).

Item 20. A method according to any of items 16 to 20, the method comprising verifying integrity of the session data (303).

### Second Item Set:

Item 1. An intraoral scanning device (2) configured to acquire intraoral scan data from a three-dimensional dental object during a scanning session, the intraoral scanning device comprising:
a processing unit (2) configured to process intraoral scan data of a patient and provide 2D image data and/or 3D image data;
a wireless interface (4) configured to transmit the 2D image data and/or the 3D image data; and
a memory (3),
   characterized in that the processing unit (4) may be configured to:
receive a linking request (410,411) for a session via the wireless interface (8);
obtain a session identifier (180);
transmit, via the wireless interface (8), a linking response (412) comprising an intraoral scanning device identifier (112) and the session identifier (180);
receive, via the wireless interface (180), an authentication message (420,421) comprising an authentication key identifier (166) and client device data (109);
select an intraoral scanning device key from a plurality of intraoral scanning device keys in the memory unit (6) based on the authentication key identifier (166);
verify the client device data (109) based on the selected intraoral scanning device key; and
terminate the session if the verification fails.

Item 2. An intraoral scanning device according to item 1, wherein the authentication message (420, 421) comprises an authentication type identifier (168), and wherein to select an intraoral scanning device key from a plurality of intraoral scanning device keys is based on the authentication type identifier (168).

Item 3. An intraoral scanning device according to any of items 1-2, wherein the client device data (109) comprises an encrypted client device certificate (106); and wherein the processing unit (4) may be configured to generate a certificate key based on a common secret; and wherein to verify the client device data (109) comprises to decrypt the encrypted client device certificate (106A) with the certificate key to obtain a decrypted version (106B) of the encrypted client device certificate.

Item 4. An intraoral scanning device according to item 3, wherein the common secret is based on the selected intraoral scanning device key and/or the session identifier (180).

Item 5. An intraoral scanning device according to any of items 3-4, wherein to verify the client device data (109) comprises to determine if the authentication key identifier (166) matches a client device key identifier (159) of the client device certificate (106), and wherein verification fails if no match is determined.

Item 6. An intraoral scanning device according to any of items 3-5, wherein to verify the client device data (109) comprises to determine if a client device type identifier (156) of the client device certificate (106) is valid and wherein verification fails if the client device type identifier (156) of the client device certificate (106) is not valid.

Item 7. An intraoral scanning device according to any of items 3-6, wherein to verify the client device data (109) comprises to verify a digital signature of the client device certificate (106), and wherein verification fails if the digital signature is not verified.

Item 8. An intraoral scanning device according to any of items 3-7, wherein the client device certificate (106) comprises a signing device identifier and/or a client device identifier (158), and wherein to verify the client device data (109) comprises to determine if the signing device identifier and/or the client device identifier (158) is valid and wherein verification fails if the client device identifier (158) of the client device (10) and/or the signing device identifier is not valid.

Item 9. An intraoral scanning device according to any of items 3-8, wherein the processing unit (4) may be configured to receive an additional authentication message (440) comprising client device data (109) and an authentication device identifier (169), wherein the processing unit (4) may be configured to
obtain, from the memory unit (6), the common secret based on the authentication device identifier (169);
generate an additional certificate key from the common secret; and
verify the client device data (109) based on the additional certificate key.

Item 10. An intraoral scanning device according to item 9, wherein the processing unit (4) may be configured to generate an offline session key based on the common secret and the session identifier (180), and wherein the processing unit (4) may be configured to communicate with the client device (10) using the offline session key.

Item 11. An intraoral scanning device according to any of items 3-10, wherein the authentication message (420, 421) comprises an authentication token identifier, and wherein the processing unit may be configured to store the authentication token identifier in the memory unit and to link the authentication token identifier with the common secret.

Item 12. An intraoral scanning device according to any of items 3-11, wherein the processing unit (4) may be configured to receive further authentication message (450) comprising client device data (109), an authentication type identifier (168), an authentication key identifier (166) and/or an authentication session token identifier (167), wherein the processing unit (4) may be configured to
obtain a common secret based on the authentication type identifier (168);
generate a token key based on the common secret;
generate a session token identifier based on the token key and the session identifier; and
verify the authentication session token identifier (167) based on the session token identifier.

Item 13. An intraoral scanning device according to any of the preceding items, wherein the processing unit (4) may be configured to generate a session key based on the session identifier (180) and the intraoral scanning device key, and wherein the processing unit (4) may be configured to receive and authenticate session data based on the session key.

Item 14. A method (500) of operating an intraoral scanning device (2) for acquiring intraoral scan data from a three-dimensional dental object during a scanning session, the intraoral scanning device comprising a processing unit (2) configured to process intraoral scan data of a patient and provide 2D image data and/or 3D image data: a memory unit (6); and a wireless interface (4) configured for transmitting the 2D image data and/or the 3D image data, characterized in that the method comprises the steps of:
receiving (A1) a linking request for a session via the wireless interface (8);
obtaining (A2) a session identifier (180);
transmitting (A3), via the wireless interface, a linking response comprising an intraoral scanning device identifier (112) and the session identifier (180);
receiving (A4), via the wireless interface (8), an authentication message (420, 421) comprising an authentication key identifier (166) and client device data (109);
selecting (A5) an intraoral scanning device key from a plurality of intraoral scanning device keys based on the authentication key identifier (166);
verifying (A6) the client device data (109) based on the selected intraoral scanning device key; and
terminating (A7) the session if verification fails.

Item 15. A method according to claim 14, wherein the authentication message (420, 421) comprises an authentication type identifier (166), and wherein selecting an intraoral scanning device key from a plurality of intraoral scanning device keys is based on the authentication type identifier (168).

### Third Item Set:

Item 1. A method (200) of securing communication for a user application installed on an external device of a dental system comprising an intraoral scanning device, a server device, and the external device, wherein securing communication for the user application comprises:
obtaining (202) challenge data in the server device;
transmitting (204) the challenge data from the server device to the user application installed on the external device;
transmitting (206) a challenge request comprising the challenge data from the user application to the intraoral scanning device;
receiving (208) a challenge response comprising response data from the intraoral scanning device;
forwarding (209) the response data from the user application to the server device;
verifying (210) the response data in the server device based on the challenge data; and
approving (212) the user application in the server device if verifying the response data is successful.

Item 2. Method according to Item 1, wherein the method comprises determining the response data in the intraoral scanning device based on the challenge data and an intraoral scanning device identifier of the intraoral scanning device.

Item 3. Method according to any of items 1-2, wherein the response data comprises or is indicative of an intraoral scanning device identifier.

Item 4. Method according to any of items 1-3, wherein receiving a challenge response comprising response data from the intraoral scanning device is performed by the user application.

Item 5. Method according to any of items 1-4, wherein approving the user application comprises setting a user application status identifier to a value indicative of the user application being approved.

Item 6. Method according to any of items 1-5, the method comprising setting a user application status identifier to a value indicative of the user application not being approved if verifying the response data fails.

Item 7. Method according to any of items 1-6, wherein the method comprises linking the user application to an intraoral scanning device in a memory of the server device if verifying the response data is successful.

Item 8. Method according to any of items 1-7, the method comprising transmitting a request for challenge data from the user application.

Item 9. Method according to item 8, wherein the request for challenge data is transmitted if a first approval criterion is fulfilled.

Item 10. Method according to any of items 1-9, the method comprising storing an approval timestamp indicative of time of last approval: determining if a second approval criterion based on the approval timestamp is fulfilled; and initiate securing communication for the user application if the second approval criterion is fulfilled.

Item 11. Method according to any of items 1-10, wherein approving the user application comprises transmitting intraoral scanning device settings specific for the intraoral scanning device to the user application.

Item 12. Method according to any of items 1-11, wherein obtaining challenge data comprises storing the challenge data in the server device, or wherein verifying the response data in the server device based on the challenge data comprises calculating the challenge data.

Item 13. A dental system (2) comprising a server device (4) and an intraoral scanning device system, said intraoral scanning device system comprising an external device (10) and an intraoral scanning device (8), the server device (4) being configured for securing communication for a user application (12) installed on the external device (10), wherein the server device (4) is configured to approve the user application (12), wherein to approve the user application (12) comprises to:
obtain challenge data;
transmit the challenge data to the user application (12);
receive a response message comprising response data from the user application (12), the response data comprising an intraoral scanning device identifier;
verify the response data based on the challenge data; and
approve the user application (12) if the response data are verified, and
   the external device (10) comprising
a processing unit;
a memory unit; and
a wireless interface,
   wherein the user application (12) is configured to secure communication for the user application, and wherein to secure communication for the user application comprises to:
obtain challenge data from the server device (4);
transmit a challenge request comprising the challenge data to the intraoral scanning device (8) of the intraoral scanning device system (2);
receive a challenge response comprising response data from the intraoral scanning device (8); and
forward the response data to the server device (4).

Item 14. Dental system (2) according to item 13, wherein the server device (4) is configured to determine if an approval criterion is fulfilled, the server device (4) being configured to initiate securing communication for the user application (12) if the approval criterion is fulfilled, wherein the approval criterion comprises a first approval criterion and a second approval criterion, and wherein the approval criterion is fulfilled if the first approval criterion or the second approval criterion is fulfilled.

Item 15. Dental system (2) according to any of items 13-14, wherein the user application (12) is configured to determine if a first approval criterion is fulfilled and to initiate securing communication for the user application if the first approval criterion is fulfilled, and wherein to obtain challenge data comprises to transmit a request for challenge data to the server device.

### Fourth Item Set:

Item 1. A method of controlling access of a client device (42) to a service of an intraoral scanning device (10), the method comprising the steps of:
requesting access of the client device (42) to the service of the intraoral scanning device (10) by providing a client device authenticator to the intraoral scanning device (10);
authenticating the client device (42) based on a validation of the provided client device authenticator by the intraoral scanning device (10);
   characterized in that
upon successful authentication, comparing a security level associated with the service requested by the client device (42) with a highest security level assigned to the client device (42) by the intraoral scanning device (10), wherein the security level is selected from a plurality of hierarchically structured security levels, and
granting access of the client device (42) to the service of the intraoral scanning device (10), if the requested security level is below or equal to the highest security level assigned to the client device (42).

Item 2. The method of item 1, wherein providing a client device authenticator comprises granting a authorization to each client device (42) and storing intraoral scanning device (10) service authorizations granted to client devices (42) on the intraoral scanning device (10); wherein the intraoral scanning device (10) rejects the access to the requested intraoral scanning device service, if the security level assigned to the client device (42) is not at least as high as the security level associated with the service request, wherein an authorization comprises at least the client device authenticator and the highest security level assigned to the client device (42), and wherein a client device privileged by an authorization to access a certain security level is also privileged to access all security levels below it.

Item 3. The method of item 2, further comprising: defining a plurality of authorization methods and assigning to each of the security levels at least one of the authorization methods in such a manner that each authorization method assigned to a certain security level is different to the authorization methods assigned to the other security levels, wherein each authorization method is for granting an authorization to a client device (42) to access intraoral scanning device service(s) assigned with at the respective security level.

Item 4. The method of item 3, wherein at least one of the authorization methods allows a user to grant authorizations autonomously by acting on the intraoral scanning device (10) or an external device communicating with the intraoral scanning device (10), without a further device being involved.

Item 5. The method of item 4, wherein the authorization methods comprise performing, by the user, at least one selective gesture on a user interface of the intraoral scanning device (10) or on an external device, such as a computer, communicating with the intraoral scanning device (10).

Item 6. The method of item 5, wherein the external device is trusted by the intraoral scanning device (10).

Item 7. The method of one of items 5 and 6, wherein the authorization methods comprise a plurality of the gestures performed by the user, wherein each of the gestures is specific for a different one of the security levels.

Item 8. The method of one of items 5 to 7, wherein the user gesture is performed in response to an authorization request received by the intraoral scanning device (10) from the client device (42).

Item 9. The method of item 8, wherein the client device (42) is authenticated by the user prior to performing the user gesture.

Item 10. The method of one of items 8 and 9, wherein the intraoral scanning device (10) provides a haptic or a visual/optical notification concerning receipt of the authorization request by the intraoral scanning device (10) to the user which includes information concerning the security level(s) to which access is requested by the client device (42).

Item 11. The method of one of items 5 to 7, wherein the user gesture is performed in response to an authorization request received by the user from the client device (42), wherein the user gesture causes to the intraoral scanning device (10) enter an authorization accept state in which it accepts an authorization request from any client device (42), and wherein the user then causes the client device (42) to send an authorization request to the intraoral scanning device (10).

Item 12. The method of item 11, wherein the intraoral scanning device (10), when being in the authorization accept state, notifies the user concerning the security levels which will be accessible to client devices (42) requesting authorization in the authorization accept state.

Item 13. The method of one of items 11 and 12, wherein the intraoral scanning device (10) notifies the user that authorization has been granted and to which client device (42) the authorization has been granted.

Item 14. The method of item 13, wherein the intraoral scanning device (10) enables the user to withdraw the granted authorization within a given time period after the notification of the grant of the authorization.

Item 15. The method of one of items 11 to 14, wherein there is a plurality of different user gestures, each of which causes the intraoral scanning device (10) to enter a different authorization accept state with different maximum accessible security level.

Item 16. The method of one of items 3 to 15, wherein the authorization methods comprise authorization by an authorization service (46), wherein the client device identifies itself to the authorization service (46) and requests authorization for access to at least one intraoral scanning device service from the authorization service (46), wherein the authorization service (46), based on the identity of the client device deciding to grant or refuse the requested authorization, wherein the authorization service (46), when grating the requested authorization, issues a token to the client device (42) including the maximum security level accessible by the client device (42), wherein the client device presents the token to the intraoral scanning device (10), wherein a trusted relation is established between the intraoral scanning device (10) and the authorization service (46), and wherein the intraoral scanning device (10), when successfully authenticating the token as having been issued by the authorization service (46), grants the requested authorization to the client device.

Item 17. The method of item 16, wherein the token is a digital certificate issued by the authorization service (46) to the client device (42).

Item 18. The method of one of the preceding items, wherein the client device authenticator contains a secret shared between the client device (42) and the intraoral scanning device (10).

Item 19. The method of one of the preceding items, wherein the security levels are represented by numerical values, with the order of the numerical values being correlated with the hierarchy of the security levels.

Item 20. The method of one of the preceding items, wherein a call dispatching table is stored on the intraoral scanning device (10) for assigning each intraoral scanning device service callable by a client device (42) to one of the security levels.

Item 21. The method of one of the preceding items, wherein the client devices (42) are grouped based on the highest security level accessible by the client device (42), with each group being assigned with the respective higher highest security level accessible by the client devices (42) of the group, and wherein the intraoral scanning device (10) rejects access to the requested intraoral scanning device service if the security level associated with the requested intraoral scanning device service is higher than the security level of the group of the client device (42).

Item 22. The method of one of the preceding items, wherein the intraoral scanning device (10) is configured to acquire intraoral scan data from a three-dimensional dental object during a scanning session, and the intraoral scanning device includes a processing unit configured to process intraoral scan data and provide 2D image data and/or 3D image data.

### Fifth Item Set:

Item 1. A client device (10) configured to intraoral scanning device communication, the client device (10) comprising
a wireless interface (8) configured to receive 2D image data and/or 3D image data from an intraoral scanning device,
a memory unit (6);
a processing unit (4) configured to process the received 2D image data and/or 3D image data:
   the memory unit (6) having a client device key (182) and a client device certificate (106, 107) stored thereon,
   wherein the processing unit (4) is configured to:
receive a connection response (412) comprising an intraoral scanning device identifier (112) via the wireless interface (8);
generate one or more keys including a certificate key based on the intraoral scanning device identifier (112) and the client device key (182);
obtain an authentication message (424) based on the certificate key and the client device certificate (106, 107), and
transmit the authentication message (424) via the wireless interface (8).

Item 2. Client device according to item 1, wherein the processing unit (4) is configured to obtain a session identifier (180), and wherein to generate one or more keys comprises to generate an intraoral scanning device key based on the intraoral scanning device identifier (112) and the client device key (182), and to generate a common secret based on the intraoral scanning device key and the session identifier (180).

Item 3. Client device according to item 2, wherein the certificate key is based on the common secret and a certificate value.

Item 4. Client device according to any of items 2-3, wherein to generate one or more keys comprises to generate a session key (188) based on the intraoral scanning device identifier (112), the session identifier (180) and the client device key (182), and wherein the processing unit (4) is configured to transmit the session key (188) to a customization device (14).

Item 5. Client device according to item 4, wherein the session key (188) is based on the common secret and a session value.

Item 6. Client device according to any of the preceding items, wherein the processing unit (4) is configured to include an authentication key identifier (166) indicative of the client device key (182) in the authentication message (424).

Item 7. Client device according to any of the preceding items, wherein the processing unit (4) is configured to include an authentication type identifier (168) in the authentication message (424).

Item 8. Client device according to any of the preceding items, wherein the client device certificate (106, 107) comprises one or more of:
a certificate type identifier (130);
a signing device identifier (136);
a client device type identifier (156);
a client device identifier (158);
a client device key identifier (159);
one or more hardware identifiers (148); and
a digital signature (113).

Item 9. A method of operating a client device for intraoral scanning device communication, the client device comprising a memory unit (6) having a client device key (182) and a client device certificate (106, 107) stored thereon, the method comprising:
receiving (S1) a connection response (412) comprising an intraoral scanning device identifier (112) via the wireless interface;
generating (S2) one or more keys including a certificate key based on the intraoral scanning device identifier (112) and the client device key (182);
obtaining (S3) an authentication message (424) based on the certificate key and the client device certificate (106, 107), and
transmitting (S4) the authentication message (424) via the wireless interface (8).

Item 10. Method according to item 9, the method comprising obtaining (S11) a session identifier (180), and wherein generating (S2) one or more keys comprises generating (S21) an intraoral scanning device key based on the intraoral scanning device identifier (112) and the client device key (182) and generating (S22) a common secret based on the intraoral scanning device key and the session identifier (180).

Item 11. Method according to item 10, wherein the certificate key is based on the common secret and a certificate value.

Item 12. Method according to any of items 10-11, wherein generating (S2) one or more keys comprises generating (S23) a session key (188) based on the intraoral scanning device identifier (112), the session identifier (180) and the client device key (182), and wherein the method comprises transmitting (S5) the session key (188) to a customization device (14).

Item 13. Method according to item 12, wherein the session key (188) is based on the common secret and a session value.

Item 14. Method according to any of items 9-13, wherein obtaining (S3) the authentication message (424) comprises including (S32) an authentication key identifier (166) indicative of the client device key (182) in the authentication message (424).

Item 15. Method according to any of items 9-14, wherein obtaining (S3) the authentication message (424) comprises including (S33) an authentication type identifier (168) in the authentication message (424).

1. A handheld intraoral scanning device for acquiring intraoral scan data from a three-dimensional dental object during a scanning session, the handheld intraoral scanning device comprising:
a processing unit configured to process intraoral scan data of a patient and provide 3D image data;
a wireless interface configured to transmit the 3D image data; and
a memory
wherein the processing unit is configured to:
receive a mode request via the wireless interface when no 3D image data is transmitted, wherein the mode request is one or more of a service mode request for a service mode, a customization mode request for customizing a user interface of the handheld intraoral scanning device; an upgrade mode request for upgrading the handheld intraoral scanning device, and a debug mode request, wherein the service mode is characterized in that a firmware part of the memory is writable;
authenticate the mode request to confirm that the mode request is valid for the handheld intraoral scanning device; and
place the handheld intraoral scanning device into the requested mode if authentication of the mode request succeeds.

2. A handheld intraoral scanning device according to claim 1, wherein the processing unit is configured to place the intraoral scanning device into a default mode if authentication of the mode request fails.

3. A handheld intraoral scanning device according to claim 2, wherein the default mode comprises booting the handheld intraoral scanning device and operating the handheld intraoral scanning device according to operating parameters set during booting.

4. A handheld intraoral scanning device according claim 1, wherein the processing unit is configured to authenticate the mode request by authenticating the sender of the mode request.

5. A handheld intraoral scanning device according to claim 1, wherein the processing unit is configured to authenticate the mode request by verifying integrity of the mode request.

6. A handheld intraoral scanning device according to claim 1, wherein to place the handheld intraoral scanning device into the requested mode if authentication of the mode request succeeds comprises sending a mode response.

7. A handheld intraoral scanning device according to claim 1, wherein the mode request is received in a session and the processing unit is configured to terminate the session if authentication of the mode request fails.

8. A handheld intraoral scanning device according to claim 1, wherein the mode request comprises a signature, and wherein to authenticate the mode request comprises to verify the signature of the mode request.

9. A handheld intraoral scanning device according to claim 1, wherein when the handheld intraoral scanning device is in a service mode, the processing unit is configured to generate a session identifier, to transmit the session identifier via the wireless interface and to store the session identifier in the handheld intraoral scanning device.

10. A handheld intraoral scanning device according to claim 1, wherein when the handheld intraoral scanning device is in a service mode, the processing unit is configured to receive data via the wireless interface, wherein the processing unit is configured to authenticate the received data and store intraoral scanning device data in a part of the memory based on the received data if authentication of the data succeeds.

11. handheld intraoral scanning device according to claim 10, wherein the data comprises a session identifier, and wherein to authenticate the data comprises to compare the received session identifier with the session identifier stored in the handheld intraoral scanning device.

12. A handheld intraoral scanning device according to claim 10, wherein the data is received in a session and the processing unit is configured to terminate the session if authentication of the received data fails.

13. Method for configuration of a handheld intraoral scanning device comprising a processing unit configured to process intraoral scan data of a patient and provide 3D image data, a memory, and a wireless interface configured to transmit the 3D image data, the method comprising:
receiving a mode request via the wireless interface when no 3D image data is being transmitted, wherein the mode request is one or more of a service mode request for updating firmware data, a customization mode request for customizing a user interface of the handheld intraoral scanning device, an upgrade mode request for upgrading the handheld intraoral scanning device, and a debug mode request, and wherein the service mode is characterized in that a firmware part of the memory is writable;
authenticating the mode request to confirm that the mode request is valid for the handheld intraoral scanning device; and
placing the handheld intraoral scanning device into the requested mode if authentication of the mode request succeeds.

14. Method according to claim 13, the method comprising placing the handheld intraoral scanning device into a default mode if authentication of the mode request fails.

15. Method according to claim 13, wherein authenticating the mode request comprises authenticating the sender of the mode request.

16. Method according to claim 13, wherein authenticating the mode request comprises verifying integrity of the mode request.

17. Method according to claim 12, wherein when the handheld intraoral scanning device is in a service mode, the method comprises:
receiving data via the wireless interface,
authenticating the received data; and
storing intraoral scanning device data in a part of the memory based on the received data if authentication of the data succeeds.
